SEQUENCE LISTING (21 sequences)

SEQ ID NO: 1 - protein, 10 aa, synthetic construct
GFTFSSYAMS

SEQ ID NO: 2 - protein, 17 aa, synthetic construct
AINASGTRTYYADSVKG

SEQ ID NO: 3 - protein, 17 aa, synthetic construct
GKGNTHKPYGYVRYFDV

SEQ ID NO: 4 - protein, 126 aa, Homo sapiens [sequence omitted]

SEQ ID NO: 5 - protein, 12 aa, synthetic construct
RASQSVSSSYLA

SEQ ID NO: 6 - protein, 7 aa, synthetic construct
GASSRAT

SEQ ID NO: 7 - protein, 9 aa, synthetic construct
LQIYNMPIT

SEQ ID NO: 8 - protein, 108 aa, Homo sapiens [sequence omitted]

SEQ ID NO: 9 - protein, 5 aa, Oryctolagus cuniculus
SYAMS

SEQ ID NO: 10 - protein, 16 aa, synthetic construct
  REGION 1..16: note = 299-000 HVR-H2 Kabat G65S
YIWSGGSTDYASWAKS

SEQ ID NO: 11 - protein, 17 aa, synthetic construct
  REGION 1..17: note = 299-000 HVR-H3 DASG
RYGTSYPDYGDASGFDP

SEQ ID NO: 12 - protein, 122 aa, synthetic construct
  REGION 1..122: note = 299-023 VH humanization variant_DANG
QSMQESGPGLVKPSQTLSLTCTVSGFSLSSYAMSWIRQHPGKGLEWIGYIWSGGSTDYASWAKSRVTISKTSTTVSLKLSSVTAADTAVYYCARRYGTSYPDYGDASGFDPWGQGTLVTVSS

SEQ ID NO: 13 - protein, 11 aa, synthetic construct
  REGION 1..11: note = 299-000 HVR-L1 RAA
RASQSISSYLA

SEQ ID NO: 14 - protein, 7 aa, Oryctolagus cuniculus
RASTLAS

SEQ ID NO: 15 - protein, 12 aa, synthetic construct
  REGION 1..12: note = 299-000 HVR-L3 NYA
QQNYASSNVDNT

SEQ ID NO: 16 - protein, 110 aa, synthetic construct
  REGION 1..110: note = 299-009 VL humanization variant_NYA
AIQLTQSPSSLSASVGDRVTITCRASQSISSYLAWYQQKPGKAPKLLIYRASTLASGVPSRFSGSGSGTDFTLTISSLQPEDFATYYCQQNYASSNVDNTFGGGTKVEIK

SEQ ID NO: 17 - protein, 215 aa, synthetic construct
  REGION 1..215: note = 0015-LC1
DIVLTQSPATLSLSPGERATLSCRASQSVSSSYLAWYQQKPGQAPRLLIYGASSRATGVPARFSGSGSGTDFTLTISSLEPEDFATYYCLQIYNMPITFGQGTKVEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 18 - protein, 455 aa, synthetic construct
  REGION 1..455: note = 0015-HC1
QVELVESGGGLVQPGGSLRLSCAASGFTFSSYAMSWVRQAPGKGLEWVSAINASGTRTYYADSVKGRFTISRDNSKNTLYLQMNSLRAEDTAVYYCARGKGNTHKPYGYVRYFDVWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 19 - protein, 215 aa, synthetic construct
  REGION 1..215: note = 0015-LC2
AIQLTQSPSSLSASVGDRVTITCRASQSISSYLAWYQQKPGKAPKLLIYRASTLASGVPSRFSGSGSGTDFTLTISSLQPEDFATYYCQQNYASSNVDNTFGGGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 20 - protein, 229 aa, synthetic construct
  REGION 1..229: note = 0015-Fab
QSMQESGPGLVKPSQTLSLTCTVSGFSLSSYAMSWIRQHPGKGLEWIGYIWSGGSTDYASWAKSRVTISKTSTTVSLKLSSVTAADTAVYYCARRYGTSYPDYGDASGFDPWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 702 aa, synthetic construct
  REGION 1..702: note = 0015-HC2
QVELVESGGGLVQPGGSLRLSCAASGFTFSSYAMSWVRQAPGKGLEWVSAINASGTRTYYADSVKGRFTISRDNSKNTLYLQMNSLRAEDTAVYYCARGKGNTHKPYGYVRYFDVWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSGGGGSGGGGSGGGGSQSMQESGPGLVKPSQTLSLTCTVSGFSLSSYAMSWIRQHPGKGLEWIGYIWSGGSTDYASWAKSRVTISKTSTTVSLKLSSVTAADTAVYYCARRYGTSYPDYGDASGFDPWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2024/0417451 A1
(43) Pub. Date: Dec. 19, 2024

(54) Therapeutic Use of Bispecific Anti-Abeta/TfR Antibodies

(71) Applicant: Hoffmann-La Roche Inc., Little Falls, NJ (US)

(72) Inventors: João André ABRANTES, Basel (CH); David Andrew AGNEW, Hertford (GB); Fabien Eric ALCARAZ, Sierentz (FR); Ruth Emma CRONEY, Welwyn Graden City (GB); Carsten Harald HOFMANN, Neuenburg (DE); Paul Philipp Max JORDAN, Basel (CH); Gregory KLEIN, Hegenheim (FR); Luka KULIC, Zurich (CH); Maddalena MARCHESI, Arlesheim (CH); Hanno SVOBODA, Berlin (DE); Annamarie VOGT, Blitzingen (CH)

(73) Assignee: Hoffmann-La Roche Inc., Little Falls, NJ (US)

(21) Appl. No.: 18/677,151

(22) Filed: May 29, 2024

Related U.S. Application Data
(63) Continuation-in-part of application No. PCT/EP24/55668, filed on Mar. 5, 2024.
(63) Continuation-in-part of application No. PCT/EP23/79276, filed on Oct. 20, 2023.
(63) Continuation-in-part of application No. PCT/EP23/64623, filed on May 31, 2023.

(51) Int. Cl. C07K 16/18; A61K 39/00; A61P 25/28; C07K 16/28

(52) U.S. Cl. CPC C07K 16/18; A61P 25/28; C07K 16/2881; A61K 2039/505; C07K 2317/24

(57) ABSTRACT

Herein is reported a bispecific antibody specifically binding to human Abeta protein and human transferrin receptor (bispecific anti-Abeta/TfR antibody) as well as the use of such bispecific antibodies as a medicament in the treatment of Alzheimer's Disease, including where the bispecific antibody is administered intravenously at a dose of 0.2 mg/kg to 7.2 mg/kg once every four weeks.

### CROSS-REFERENCE TO RELATED APPLICATIONS

This application is a continuation-in-part of International Patent Application No. PCT/EP2024/055668, which was filed on Mar. 5, 2024, International Patent Application No. PCT/EP2023/079276, which was filed on Oct. 20, 2023, and International Patent Application No. PCT/EP2023/064623, which was filed on May 31, 2023, the entire contents of which are incorporated by reference herein.

### SEQUENCE LISTING

The specification further incorporates by reference the Sequence Listing submitted herewith on May 29, 2024. The Sequence Listing file, identified as 081823_0112.xml, is 25,314 bytes and was created on May 29, 2024. The Sequence Listing electronically filed herewith, does not extend beyond the scope of the specification and thus does not contain new matter.

### FIELD

The current invention is in the field of therapeutic antibodies for the treatment of neurological diseases, especially of Alzheimer's Disease. In more detail, herein is reported the therapeutic use of trontinemab, a bispecific anti-Abeta/TfR antibody that targets human Abeta protein and that has active blood-brain-barrier passage by binding to the human transferrin receptor (TFR).

### BACKGROUND

The WHO estimates that more than 55 million people worldwide are living with dementia and that 10 million new cases are diagnosed every year (World Health Organization (2023)). The total number of people with dementia is estimated to reach 78 million in 2030 and to 139 million by 2050 (Alzheimer's Disease International (2022)). Alzheimer's Disease (AD) is the most common form of dementia, accounting for 60%-70% of cases (World Health Organization 2023). The prevalence of AD increases with age, with a global prevalence of 5%-8% in people aged 60 years or over.

Although AD has been identified more than 100 years ago, despite massive efforts to develop disease-modifying therapeutics the cognitive deterioration of AD has remained frustratingly (Zhao et al., Int. J. Nanomed. 18 (2023) 7825-7845).

Alzheimer's disease is clinically characterized by a progressive impairment in cognitive abilities, which results in decreased function and gradual loss of independence (Mesterton, J., et al., Curr. Alz. Res. 7 (2010) 358-367). There is great inter-individual variability (IIV) in AD progression with survival dependent on many factors, including age at onset. In general, the clinical picture evolves from “pre-dementia” or “prodromal AD” to mild, moderate, and then severe AD. At the early stage of AD, a slight impairment of memory, language, and visuospatial function can be observed. As AD advances, patients become progressively impaired not only in terms of cognition but also in activities of daily living and the burden on caregivers significantly increases. The median survival time following a diagnosis of AD depends on the patient's age at diagnosis and ranges from 8.3 years for persons diagnosed with AD at 65 years old to 3.4 years for those 90 years old (Brookmeyer, R., et al., Arch. Neurol. 59 (2002) 1764-1767). On average, individuals live 6 years after diagnosis of AD (Helzner, E. P., et al., Neurol. 71 (2008) 1489-1495).

Because of its increasing prevalence, human burden, long duration, and high cost of care, AD is expected to continue to represent a major public health problem.

The neuropathological hallmarks of AD are extracellular amyloid plaques built of insoluble Amyloid beta (Abeta) protein and intraneuronal neurofibrillary tangles consisting of hyperphosphorylated tau filaments (Bloom, G. S., JAMA Neurol. 71 (2014) 505-508). Although the etiology of AD is not completely understood, current research suggests that Abeta protein processing and deposition play a critical role in the cascade of biological events involved in the pathogenesis of the disease Abeta protein a prime suspect of memory loss and cognitive decline in the early phase of AD. Emerging evidence from several independent amyloid clearing mAbs has identified a relationship between Abeta plaque reduction and an improvement in cognition (see, e.g., Selkoe, D. J. and Hardy, J., EMBO Molec. Med. 8 (2016) 595-608; Kulic, L., et al. Presented at AD/PD 2021, virtual conference). Abeta protein is derived from proteolytic processing of the amyloid beta precursor protein (APP). This protein exists in two major forms: Abeta1-40 and Abeta1-42 (Citron, M., Trends Pharmacol. Sci. 25 (2004) 92-97). The accumulation in the brain begins up to 20 years before the occurrence of clinical dementia in AD and causes a series of downstream events leading to synaptic dysfunction, inflammation, neurodegeneration, and clinical symptoms (Selkoe, D. J. and Hardy, J., EMBO Mol.). Consequently, therapies targeting this process and especially Abeta protein have the potential to significantly alter disease progression.

Unfortunately, Abeta protein, i.e., therapeutic target, engagement is associated with adverse events. In more detail, recently approved standard monoclonal anti-Abeta antibodies show effective amyloid lowering only within 1.5 years of treatment and a modest 27-35% delay of progression with an ARIA rate of 13-25%. Thus, there is a high unmet need for more efficacious, safer and more convenient treatment.

Zhao et al. reported that the recent successive approval of anti-amyloid-O monoclonal antibodies as disease-modifying therapies against Alzheimer's disease has raised great confidence in the development of anti-Alzheimer's disease therapies. However, the current therapies still face the dilemma of significant adverse reactions and limited effects (Int. J. Nanomed. 18 (2023) 7825-7845).

The current opinion in the art is that a rapid and efficient clearance of aggregated forms of Abeta protein is a prerequisite to achieve significant clinical effect.

### SUMMARY OF THE INVENTION

The above drawbacks of current anti-Abeta protein therapies are overcome with the current invention.

The current invention is based, at least in part, on the finding that the intravenous administration of trontinemab at a dose of 1.8 mg/kg once every four weeks results in a mean change from baseline in amyloid PET Centiloids by nominal visit already after administration of nominal Dose 3 of −65.3 Centiloids (range from −102.8 to −42.6) (mean number of doses received: of 2.8).

Thus, it has been found that rapid amyloid plaque (Abeta protein plaque) clearance can be achieved at surprisingly and significantly lower dose levels than with standard typical anti-Abeta monoclonal antibodies using the transferrin receptor-based (TfR-based) Brainshuttle™ approach of trontinemab. It has been found that the Brainshuttle™ technology enables a higher brain exposure and broader CNS distribution compared to a standard IgG, i.e., non-Brainshuttle™, antibodies.

Therefore, trontinemab, along with its structural and functional equivalents, can be the best-in-disease amyloid-lowering treatment with superior efficacy, safety, and convenience versus standard antibodies by slowing speed of disease progression, e.g., at least by half, and at a relatively low risk of ARIA. For example, one aspect of the current invention is directed to a bispecific antibody specifically binding to human Abeta protein and human transferrin receptor (bispecific anti-Abeta/TfR antibody) for use as a medicament in the treatment of Alzheimer's Disease, wherein the antibody is administered intravenously at a dose of 0.2 mg/kg to 7.2 mg/kg once every four weeks.

One exemplary aspect of the current invention is directed to a pharmaceutical composition for treating prodromal to mild Alzheimer's disease (AD), comprising administering to a patient with prodromal to mild Alzheimer's disease at a once every four weeks intravenous dose of 0.2 mg/kg to 7.2 mg/kg of a bispecific anti-Abeta/TfR antibody.

One exemplary aspect of the current invention is directed to a bispecific anti-Abeta/TfR antibody for use in the treatment of Alzheimer's Disease, wherein the antibody is administered intravenously at a dose of 0.2 mg/kg to 7.2 mg/kg once every four weeks.

In certain embodiments, administration of the bispecific anti-Abeta/TfR antibody composition results in at least 30% of subjects being amyloid negative as determined by visual reads of amyloid PET images after 3 months of administration (3 doses) of the antibody composition. Accordingly, in certain embodiments, the administration of the antibody composition results in a subject being determined, by visual reads of amyloid PET images after 3 months of administration (3 doses) of the antibody composition, to be amyloid negative. Preferably, 30 to 70% of subjects being amyloid negative as determined by visual reads of amyloid PET images after 3 to 6 months of administration (3 to 6 doses) of the antibody composition. Accordingly, in certain embodiments, the administration of the antibody composition results in a subject being determined, by visual reads of amyloid PET images after 3 to 6 months of administration (3 to 6 doses) of the antibody composition, to be amyloid negative.

In one exemplary embodiment, administration of the antibody composition results in at least 70% of subjects being amyloid negative as determined by visual reads of amyloid PET images after 4 to 6 months of administration (3 to 6 doses) of the antibody composition. Accordingly, in certain embodiments, the administration of the antibody composition results in a subject being determined, by visual reads of amyloid PET images after 4 to 6 months of administration (3 to 6 doses) of the antibody composition, to be amyloid negative

In certain embodiments, the bispecific anti-Abeta/TfR antibody comprises:
i) two copies of a first pair of an antibody heavy chain variable domain and a cognate antibody light chain variable domains forming a binding site specifically binding to human Abeta protein fragment Abeta1-42 comprising:
   three heavy chain complementarity determining regions (HCDRs) comprising amino acid sequences of SEQ ID NO: 1 (HCDR1), SEQ ID NO: 2 (HCDR2), and SEQ ID NO: 3 (HCDR3); and
   three light chain complementarity determining regions (LCDRs) comprising amino acid sequences of SEQ ID NO: 5 (LCDR1), SEQ ID NO: 6 (LCDR2), and SEQ ID NO: 7 (LCDR3); and
ii) one copy of a second pair of an antibody heavy chain variable domain and a cognate antibody light chain variable domains forming a binding site specifically binding to human transferrin receptor 1 comprising
   three heavy chain complementarity determining regions (HCDRs) comprising amino acid sequences of SEQ ID NO: 9 (HCDR1), SEQ ID NO: 10 (HCDR2), and SEQ ID NO: 11 (HCDR3); and
   three light chain complementarity determining regions (LCDRs) comprising amino acid sequences of SEQ ID NO: 13 (LCDR1), SEQ ID NO: 14 (LCDR2), and SEQ ID NO: 15 (LCDR3).

In certain embodiments, the bispecific anti-Abeta/TfR antibody is trontinemab (RG6102). In certain embodiments, trontinemab uses receptor-mediated transcytosis for crossing the blood-brain-barrier. In certain embodiments, Trontinemab uses the transcellular pathway for crossing the blood-brain-barrier.

In certain embodiments, the treatment is of a subject that has been diagnosed as having mild Alzheimer's disease.

In certain embodiments, the treatment is of a subject that has been diagnosed as having mild-to-moderate Alzheimer's disease.

In certain embodiments, the treatment is of a subject that has been diagnosed as having prodromal Alzheimer's disease.

In certain embodiments, the administration of the bispecific anti-Abeta/TfR antibody reduces the amyloid level from amyloid positive to amyloid negative as determined by PET imaging within 12 weeks. The PET imaging is, in certain embodiments, performed with florbetapir or florbetaben. The administration is, in certain embodiments, performed once every four weeks (Q4W). The administration is, in certain embodiments, performed intravenously. The rapid amyloid plaque clearance with trontinemab is achieved, in certain embodiments, at significantly lower dose levels than with typical anti-amyloid monoclonal antibodies. The typical anti-amyloid antibodies are, in certain embodiments, aducanumab or/and lecanemab.

In certain embodiments, the administration of the bispecific anti-Abeta/TfR antibody reduces the amyloid level from 50 Centiloids or more, e.g., as determined by PET imaging before the start of the administration, to 24 Centiloids or less, e.g., as determined with the same PET imaging within 12 weeks after start of the administration. The PET imaging is, in certain embodiments, performed with florbetapir or florbetaben. The administration is, in certain embodiments, performed once every four weeks (Q4W). The administration is, in certain embodiments, performed intravenously. The rapid amyloid plaque clearance is achieved, in certain embodiments, at significantly lower dose levels than with typical anti-amyloid monoclonal antibodies. The typical anti-amyloid antibodies are, in certain embodiments, aducanumab or/and lecanemab.

In certain embodiments, the administration of the bispecific anti-Abeta/TfR antibody reduces the amyloid level by at least 40 Centiloids relative to placebo as determined by visual reads of amyloid PET.

In certain embodiments, the administration of the bispecific anti-Abeta/TfR antibody reduces the amyloid level within 3 months after the start of the administration of the antibody.

In certain embodiments, no further Alzheimer's disease medication other than trontinemab is administered.

In certain embodiments, the bispecific anti-Abeta/TfR antibody is administered at a dose of 1.8 mg/kg to 7.2 mg/kg.

In certain embodiments, the bispecific anti-Abeta/TfR antibody is administered at a dose of 1.8 mg/kg to 3.6 mg/kg.

In certain embodiments, Abeta is human Abeta protein fragment 1-42 and transferrin receptor is human transfer receptor 1.

In certain embodiments, the bispecific anti-Abeta/TfR antibody is after a number of administrations of the dose continued to be administered at a lower dose of 0.2 mg/kg to 3.6 mg/kg relative to the weight of the subject. In one embodiment, the number of administrations is 3 to 60.

In certain embodiments, the administration of the bispecific anti-Abeta/TfR antibody has a low level of drug-related adverse events. For example, but not by way of limitation, in certain embodiments the level of drug-related adverse events can be/is reduced as compared to aducanumab or/and lecanemab. In certain embodiments, the drug-related adverse event is amyloid-related imaging abnormalities (ARIA). In certain embodiments, the low level is 10% or less of treated patients.

Thus, the invention encompasses at least the following embodiments:
1. A method of reducing amyloid level in a subject having early Alzheimer's disease comprising administering a composition comprising a therapeutically effective amount of or less than 9 mg/kg relative to the weight of the subject once every four weeks of at least one anti-Abeta antibody.
2. The method according to embodiment 1, wherein the at least one anti-Abeta antibody comprises a pair of an antibody heavy chain variable domain and a cognate antibody light chain variable domains forming a binding site specifically binding to human Abeta protein comprising
   three heavy chain complementarity determining regions (HCDRs) comprising amino acid sequences of SEQ ID NO: 1 (HCDR1), SEQ ID NO: 2 (HCDR2), and SEQ ID NO: 3 (HCDR3); and
   three light chain complementarity determining regions (LCDRs) comprising amino acid sequences of SEQ ID NO: 5 (LCDR1), SEQ ID NO: 6 (LCDR2), and SEQ ID NO: 7 (LCDR3).
3. The method according to any one of embodiments 1 to 2, wherein the at least one anti-Abeta antibody is a bispecific anti-Abeta/TfR antibody.
4. The method according to embodiment 3, wherein the anti-Abeta/TfR antibody comprises
   two pairs each of an antibody heavy chain variable domain and a cognate antibody light chain variable domains forming a binding site specifically binding to human Abeta protein comprising
      three heavy chain complementarity determining regions (HCDRs) comprising amino acid sequences of SEQ ID NO: 1 (HCDR1), SEQ ID NO: 2 (HCDR2), and SEQ ID NO: 3 (HCDR3); and
      three light chain complementarity determining regions (LCDRs) comprising amino acid sequences of SEQ ID NO: 5 (LCDR1), SEQ ID NO: 6 (LCDR2), and SEQ ID NO: 7 (LCDR3).
   one pair of an antibody heavy chain variable domain and a cognate antibody light chain variable domains forming a binding site specifically binding to human TfR comprising
      three heavy chain complementarity determining regions (HCDRs) comprising amino acid sequences of SEQ ID NO: 9 (HCDR1), SEQ ID NO: 10 (HCDR2), and SEQ ID NO: 11 (HCDR3); and
      three light chain complementarity determining regions (LCDRs) comprising amino acid sequences of SEQ ID NO: 13 (LCDR1), SEQ ID NO: 14 (LCDR2), and SEQ ID NO: 15 (LCDR3).
5. The method according to any one of embodiments 1 to 4, wherein the at least one anti-Abeta antibody is trontinemab.
6. A method of reducing amyloid level in a subject having early Alzheimer's disease comprising administering a composition comprising a therapeutically effective amount of the bispecific anti-Abeta/TfR antibody trontinemab.
7. The method according to embodiment 6, wherein the therapeutically effective amount of the bispecific anti-Abeta/TfR antibody trontinemab is 9 mg/kg or less relative to the weight of the subject.
8. The method according to any one of embodiments 6 to 7, wherein the therapeutically effective amount of the anti-Abeta antibody trontinemab is administered once every four weeks.
9. A method of reducing amyloid level in a subject having early Alzheimer's disease comprising administering a composition comprising a therapeutically effective amount of at least one bispecific anti-Abeta/TfR antibody, wherein the therapeutically effective amount results in a lower number of ARIA events compared to a monospecific anti-Abeta antibody, preferably than aducanumab or/and lecanemab.
10. The method according to embodiment 9, wherein the at least one bispecific anti-Abeta/TfR antibody comprises
   i) two copies of a first pair of an antibody heavy chain variable domain and a cognate antibody light chain variable domains forming a binding site specifically binding to human Abeta each comprising
      three heavy chain complementarity determining regions (HCDRs) comprising amino acid sequences of SEQ ID NO: 1 (HCDR1), SEQ ID NO: 2 (HCDR2), and SEQ ID NO: 3 (HCDR3); and
      three light chain complementarity determining regions (LCDRs) comprising amino acid sequences of SEQ ID NO: 5 (LCDR1), SEQ ID NO: 6 (LCDR2), and SEQ ID NO: 7 (LCDR3); and
   ii) one copy of a second pair of an antibody heavy chain variable domain and a cognate antibody light chain variable domains forming a binding site specifically binding to human TfR comprising
      three heavy chain complementarity determining regions (HCDRs) comprising amino acid sequences of SEQ ID NO: 9 (HCDR1), SEQ ID NO: 10 (HCDR2), and SEQ ID NO: 11 (HCDR3); and
      three light chain complementarity determining regions (LCDRs) comprising amino acid sequences of SEQ ID NO: 13 (LCDR1), SEQ ID NO: 14 (LCDR2), and SEQ ID NO: 15 (LCDR3).
11. The method according to any one of embodiments 9 to 10, wherein the at least one bispecific anti-Abeta/TfR antibody is trontinemab.
12. The method according to any one of embodiments 9 to 11, wherein the therapeutically effective amount of the at least one bispecific anti-Abeta/TfR antibody is 9 mg/kg or less relative to the weight of the subject.
13. The method according to any one of embodiments 9 to 12, wherein the therapeutically effective amount of the at least one bispecific anti-Abeta/TfR antibody is administered once every four weeks.
14. The method according to any one of embodiments 1 to 13, wherein the at least one bispecific anti-Abeta/TfR antibody is administered intravenously.
15. The method according to any one of embodiments 1 to 14, wherein the subject having early Alzheimer's disease has been diagnosed as having mild, mild-to-prodromal or prodromal Alzheimer's disease.
16. The method according to any one of embodiments 1 to 14, wherein the subject having early Alzheimer's disease has been diagnosed as having mild cognitive impairment due to Alzheimer's disease, intermediate likelihood and/or has been diagnosed as having mild Alzheimer's disease dementia.
17. The method according to any one of embodiments 1 to 16, wherein the composition comprises 0.2 mg/kg to 7.2 mg/kg of the at least one anti-Abeta antibody relative to the weight of the subject.
18. The method according to any one of embodiments 1 to 17, wherein the composition comprises 0.6 mg/kg to 7.2 mg/kg of the at least one anti-Abeta antibody relative to the weight of the subject.
19. The method according to any one of embodiments 1 to 18, wherein the composition comprises 1.2 mg/kg to 7.2 mg/kg of the at least one anti-Abeta antibody relative to the weight of the subject.
20. The method according to any one of embodiments 1 to 19, wherein the composition comprises 1.8 mg/kg to 7.2 mg/kg of the at least one anti-Abeta antibody relative to the weight of the subject.
21. The method according to any one of embodiments 1 to 20, wherein the composition comprises 2 mg/kg to 5 mg/kg of the at least one anti-Abeta antibody relative to the weight of the subject.
22. The method according to any one of embodiments 1 to 21, wherein the composition comprises 3 mg/kg to 4 mg/kg of the at least one anti-Abeta antibody relative to the weight of the subject.
23. The method according to any one of embodiments 1 to 17, wherein the composition comprises 0.2 mg/kg to 3.6 mg/kg of the at least one anti-Abeta antibody relative to the weight of the subject.
24. The method according to any one of embodiments 1 to 17 and 23, wherein the composition comprises 0.6 mg/kg to 3.6 mg/kg of the at least one anti-Abeta antibody relative to the weight of the subject.
25. The method according to any one of embodiments 1 to 17 and 23 to 24, wherein the composition comprises 1.2 mg/kg to 3.6 mg/kg of the at least one anti-Abeta antibody relative to the weight of the subject.
26. The method according to any one of embodiments 1 to 25, wherein the composition comprises 1.8 mg/kg to 3.6 mg/kg of the at least one anti-Abeta antibody relative to the weight of the subject.
27. The method according to any one of embodiments 1 to 26, wherein the composition comprises about 1.8 mg/kg or about 1.9 mg/kg or about 2.0 mg/kg or about 2.1 mg/kg or about 2.2 mg/kg or about 2.3 mg/kg or about 2.4 mg/kg or about 2.5 mg/kg or about 2.6 mg/kg or about 2.7 mg/kg or about 2.8 mg/kg or about 2.9 mg/kg or about 3.0 mg/kg or about 3.1 mg/kg or about 3.2 mg/kg or about 3.3 mg/kg or about 3.4 mg/kg or about 3.5 mg/kg or about 3.6 mg/kg of the at least one anti-Abeta antibody relative to the weight of the subject.
28. The method according to any one of embodiments 1 to 26, wherein the composition comprises 1.2 mg/kg to 7.2 mg/kg of the at least one anti-Abeta antibody relative to the weight of the subject for a number of administrations and thereafter the composition comprises a lower amount of the at least one anti-Abeta antibody in the range of 0.2 mg/kg to 3.6 mg/kg relative to the weight of the subject.
29. The method according to embodiment 28, wherein the number of administrations is 3 to 60.
30. The method according to any one of embodiments 1 to 29, wherein the amyloid level is reduced by at least 20 Centiloids relative to placebo as determined by visual reads of amyloid PET images.
31. The method according to any one of embodiments 1 to 30, wherein the amyloid level is reduced by at least 25 Centiloids relative to placebo as determined by visual reads of amyloid PET images.
32. The method according to any one of embodiments 1 to 31, wherein the amyloid level is reduced by at least 60 Centiloids relative to placebo as determined by visual reads of amyloid PET images.
33. The method according to any one of embodiments 1 to 32, wherein the amyloid level is reduced by at least 75 Centiloids relative to placebo as determined by visual reads of amyloid PET images.
34. The method according to any one of embodiments 1 to 33, wherein the amyloid level is reduced by at least 80 Centiloids relative to baseline as determined by visual reads of amyloid PET images.
35. The method according to any one of embodiments 1 to 34, wherein the amyloid level is reduced by at least 85 Centiloids relative to placebo as determined by visual reads of amyloid PET images.
36. The method according to any one of embodiments 1 to 35, wherein the amyloid level is reduced by at least 90 Centiloids relative to placebo as determined by visual reads of amyloid PET images.
37. The method according to any one of embodiments 1 to 29, wherein the amyloid level is reduced by at least 20 Centiloids relative to baseline as determined by visual reads of amyloid PET images.
38. The method according to any one of embodiments 1 to 29 and 37, wherein the amyloid level is reduced by at least 25 Centiloids relative to baseline as determined by visual reads of amyloid PET images.
39. The method according to any one of embodiments 1 to 29 and 37 to 38, wherein the amyloid level is reduced by at least 60 Centiloids relative to baseline as determined by visual reads of amyloid PET images.
40. The method according to any one of embodiments 1 to 29 and 37 to 39, wherein the amyloid level is reduced by at least 75 Centiloids relative to baseline as determined by visual reads of amyloid PET images.
41. The method according to any one of embodiments 1 to 29 and 37 to 40, wherein the amyloid level is reduced by at least 80 Centiloids relative to baseline as determined by visual reads of amyloid PET images.
42. The method according to any one of embodiments 1 to 29 and 37 to 41, wherein the amyloid level is reduced by at least 85 Centiloids relative to baseline as determined by visual reads of amyloid PET images.
43. The method according to any one of embodiments 1 to 29 and 37 to 42, wherein the amyloid level is reduced by at least 90 Centiloids relative to baseline as determined by visual reads of amyloid PET images.
44. The method according to any one of embodiments 1 to 43, wherein the reduction of the amyloid level is after 18 months of administration of the composition.
45. The method according to any one of embodiments 1 to 44, wherein the reduction of the amyloid level is after 12 months of administration of the composition.
46. The method according to any one of embodiments 1 to 45, wherein the reduction of the amyloid level is after 6 months of administration of the composition.
47. The method according to any one of embodiments 1 to 46, wherein the reduction of the amyloid level is after 3 months of administration of the composition.
48. The method according to any one of embodiments 1 to 47, wherein said administration results in a reduction of cerebrospinal fluid level of Abeta1-42 protein fragment.
49. The method according to any one of embodiments 1 to 48, wherein the subject is concomitantly administered at least one Alzheimer's disease medication other than trontinemab.
50. The method according to any one of embodiments 1 to 48, wherein the subject is not concomitantly administered at least one Alzheimer's disease medication other than trontinemab.
51. A method of converting an amyloid positive subject having early Alzheimer's disease to amyloid negative subject comprising administering a composition comprising a therapeutically effective amount of at least one bispecific anti-Abeta/TfR antibody.
52. The method according to embodiment 51, wherein administration of the composition results in at least 30% of subjects being amyloid negative as determined by visual reads of amyloid PET images after 3 months of administration of the composition, preferably, 30 to 70% of subjects being amyloid negative as determined by visual reads of amyloid PET images after 3 months of administration (3 doses) of the composition.
52a. The method according to any one of embodiments 51 to 52, wherein administration of the composition results in at least 30% of subjects being amyloid negative as determined by visual reads of amyloid PET images after 6 months of administration of the composition, preferably, 30 to 70% of subjects being amyloid negative as determined by visual reads of amyloid PET images after 6 months of administration (3 doses) of the composition.
52b. The method according to embodiment 51, wherein the administration of the antibody composition results in a subject being determined, by visual reads of amyloid PET images after 3 months of administration of the antibody composition, to be amyloid negative.
52c. The method according to any one of embodiments 51 and 52b, wherein the administration of the antibody composition results in a subject being determined, by visual reads of amyloid PET images after 4 to 6 months of administration of the antibody composition, to be amyloid negative.
52d. The method according to any one of embodiments 51, 52b or 52c, wherein the administration of the antibody composition results in a subject being determined, by visual reads of amyloid PET images after 3 to 6 months of administration of the antibody composition, to be amyloid negative.
53. The method according to any one of embodiments 51 to 52, wherein administration of composition results in at least 50% of the subjects being amyloid negative, as determined by visual reads of amyloid PET images, after 12 months of administration of the composition.
54. The method according to any one of embodiments 51 to 53 wherein administration of the composition results in at least 70% of the subjects being amyloid negative, as determined by visual reads of amyloid PET images, after 18 months of administration of the composition.
55. The method according to any one of embodiments 51 to 54, wherein the at least one bispecific anti-Abeta/TfR antibody comprises
   i) two copies of a first pair of an antibody heavy chain variable domain and a cognate antibody light chain variable domains forming a binding site specifically binding to human Abeta protein each comprising
      three heavy chain complementarity determining regions (HCDRs) comprising amino acid sequences of SEQ ID NO: 1 (HCDR1), SEQ ID NO: 2 (HCDR2), and SEQ ID NO: 3 (HCDR3); and
      three light chain complementarity determining regions (LCDRs) comprising amino acid sequences of SEQ ID NO: 5 (LCDR1), SEQ ID NO: 6 (LCDR2), and SEQ ID NO: 7 (LCDR3); and
   ii) one copy of a second pair of an antibody heavy chain variable domain and a cognate antibody light chain variable domains forming a binding site specifically binding to human TfR comprising
      three heavy chain complementarity determining regions (HCDRs) comprising amino acid sequences of SEQ ID NO: 9 (HCDR1), SEQ ID NO: 10 (HCDR2), and SEQ ID NO: 11 (HCDR3); and
      three light chain complementarity determining regions (LCDRs) comprising amino acid sequences of SEQ ID NO: 13 (LCDR1), SEQ ID NO: 14 (LCDR2), and SEQ ID NO: 15 (LCDR3).
56. The method according to any one of embodiments 51 to 55, wherein the at least one bispecific anti-Abeta/TfR antibody is trontinemab.
57. The method according to any one of embodiments 51 to 56, wherein the therapeutically effective amount of the at least one bispecific anti-Abeta/TfR antibody is 9 mg/kg or less relative to the weight of the subject.
58. The method according to any one of embodiments 51 to 57, wherein the therapeutically effective amount of the at least one bispecific anti-Abeta/TfR antibody is administered once every four weeks.
59. The method according to any one of embodiments 51 to 58, wherein the at least one bispecific anti-Abeta/TfR antibody is administered intravenously.
60. The method according to any one of embodiments 51 to 59, wherein the subject having early Alzheimer's disease has been diagnosed as having mild or prodromal Alzheimer's disease.
61. The method according to any one of embodiments 51 to 59, wherein the subject having early Alzheimer's disease has been diagnosed as having mild cognitive impairment due to Alzheimer's disease, intermediate likelihood and/or has been diagnosed as having mild Alzheimer's disease dementia.
62. The method according to any one of embodiments 51 to 61, wherein the composition comprises 0.2 mg/kg to 7.2 mg/kg of the at least one anti-Abeta antibody relative to the weight of the subject.
63. The method according to any one of embodiments 51 to 62, wherein the composition comprises 0.6 mg/kg to 7.2 mg/kg of the at least one anti-Abeta antibody relative to the weight of the subject.
64. The method according to any one of embodiments 51 to 63, wherein the composition comprises 1.2 mg/kg to 7.2 mg/kg of the at least one anti-Abeta antibody relative to the weight of the subject.
65. The method according to any one of embodiments 51 to 64, wherein the composition comprises 1.8 mg/kg to 7.2 mg/kg of the at least one anti-Abeta antibody relative to the weight of the subject.
66. The method according to any one of embodiments 51 to 65, wherein the composition comprises 2 mg/kg to 5 mg/kg of the at least one anti-Abeta antibody relative to the weight of the subject.
67. The method according to any one of embodiments 51 to 66, wherein the composition comprises 3 mg/kg to 4 mg/kg of the at least one anti-Abeta antibody relative to the weight of the subject.
68. The method according to any one of embodiments 51 to 62, wherein the composition comprises 0.2 mg/kg to 3.6 mg/kg of the at least one anti-Abeta antibody relative to the weight of the subject.
69. The method according to any one of embodiments 51 to 62 and 68, wherein the composition comprises 0.6 mg/kg to 3.6 mg/kg of the at least one anti-Abeta antibody relative to the weight of the subject.
70. The method according to any one of embodiments 51 to 62 and 68 to 69, wherein the composition comprises 1.2 mg/kg to 3.6 mg/kg of the at least one anti-Abeta antibody relative to the weight of the subject.
71. The method according to any one of embodiments 51 to 70, wherein the composition comprises 1.8 mg/kg to 3.6 mg/kg of the at least one anti-Abeta antibody relative to the weight of the subject.
72. The method according to any one of embodiments 51 to 71, wherein the composition comprises about 1.8 mg/kg or about 1.9 mg/kg or about 2.0 mg/kg or about 2.1 mg/kg or about 2.2 mg/kg or about 2.3 mg/kg or about 2.4 mg/kg or about 2.5 mg/kg or about 2.6 mg/kg or about 2.7 mg/kg or about 2.8 mg/kg or about 2.9 mg/kg or about 3.0 mg/kg or about 3.1 mg/kg or about 3.2 mg/kg or about 3.3 mg/kg or about 3.4 mg/kg or about 3.5 mg/kg or about 3.6 mg/kg of the at least one anti-Abeta antibody relative to the weight of the subject.
73. The method according to any one of embodiments 51 to 72, wherein the composition comprises 1.2 mg/kg to 7.2 mg/kg of the at least one anti-Abeta antibody relative to the weight of the subject for a number of administrations and thereafter the composition comprises 0.2 mg/kg to 3.6 mg/kg of the at least one anti-Abeta antibody relative to the weight of the subject.
74. The method according to embodiment 73, wherein the number of administrations is 3 to 60.
75. The method according to any one of embodiments 51 to 74, wherein the amyloid level is reduced by at least 75 Centiloids relative to placebo as determined by visual reads of amyloid PET images.
76. The method according to any one of embodiments 51 to 75, wherein the amyloid level is reduced by at least 80 Centiloids relative to baseline as determined by visual reads of amyloid PET images.
77. The method according to any one of embodiments 51 to 76, wherein the amyloid level is reduced by at least 85 Centiloids relative to placebo as determined by visual reads of amyloid PET images.
78. The method according to any one of embodiments 51 to 77, wherein the amyloid level is reduced by at least 90 Centiloids relative to placebo as determined by visual reads of amyloid PET images.
79. The method according to any one of embodiments 51 to 74, wherein the amyloid level is reduced by at least 75 Centiloids relative to baseline as determined by visual reads of amyloid PET images.
80. The method according to any one of embodiments 51 to 74 and 79, wherein the amyloid level is reduced by at least 80 Centiloids relative to baseline as determined by visual reads of amyloid PET images.
81. The method according to any one of embodiments 51 to 74 and 79 to 80, wherein the amyloid level is reduced by at least 85 Centiloids relative to baseline as determined by visual reads of amyloid PET images.
82. The method according to any one of embodiments 51 to 74 and 79 to 81, wherein the amyloid level is reduced by at least 90 Centiloids relative to baseline as determined by visual reads of amyloid PET images.
83. The method according to any one of embodiments 51 to 82, wherein the reduction of the amyloid level is after 18 months of administration of the composition.
84. The method according to any one of embodiments 51 to 83, wherein the reduction of the amyloid level is after 12 months of administration of the composition.
85. The method according to any one of embodiments 51 to 84, wherein the reduction of the amyloid level is after 6 months of administration of the composition.
86. The method according to any one of embodiments 51 to 85, wherein the reduction of the amyloid level is after 3 months of administration of the composition.
87. The method according to any one of embodiments 51 to 86, wherein said administration results in a reduction of cerebrospinal fluid level of Abeta1-42 protein fragment.
88. The method according to any one of embodiments 51 to 87, wherein the subject is concomitantly administered at least one Alzheimer's disease medication other than trontinemab.
89. The method according to any one of embodiments 51 to 88, wherein the subject is not concomitantly administered at least one Alzheimer's disease medication other than trontinemab.
90. A method of treating a subject having early Alzheimer's disease comprising administering a composition comprising a therapeutically effective amount of at least one bispecific anti-Abeta/TfR antibody, wherein the severity of at least one symptom associated with Alzheimer's disease is reduced by at least 10%, 20%, 30%, 40%, 50%, 60%, 70%, 80%, 90%, or 95% relative to the severity of the same symptom in the same subject prior to treatment.
91. The method according to embodiment 90, wherein the severity of at least one symptom associated with Alzheimer's disease is reduced by at least 25% relative to placebo as determined by CDR-SB or at least 40% relative to placebo as determined by PET imaging after 18 months of administration of the composition.
92. The method according to any one of embodiments 90 to 91, wherein the severity of at least one symptom associated with Alzheimer's disease is reduced by at least 60% relative to placebo as determined by CDR-SB or at least 75% relative to placebo as determined by PET imaging after 18 months of administration of the composition.
93. The method according to any one of embodiments 90 to 92, wherein the at least one symptom associated with Alzheimer's disease is chosen from clinical decline and brain amyloid level.
94. The method according to any one of embodiments 90 to 93, wherein the at least one bispecific anti-Abeta/TfR antibody comprises
   i) two copies of a first pair of an antibody heavy chain variable domain and a cognate antibody light chain variable domains forming a binding site specifically binding to human Abeta protein each comprising
      three heavy chain complementarity determining regions (HCDRs) comprising amino acid sequences of SEQ ID NO: 1 (HCDR1), SEQ ID NO: 2 (HCDR2), and SEQ ID NO: 3 (HCDR3); and
      three light chain complementarity determining regions (LCDRs) comprising amino acid sequences of SEQ ID NO: 5 (LCDR1), SEQ ID NO: 6 (LCDR2), and SEQ ID NO: 7 (LCDR3); and
   ii) one copy of a second pair of an antibody heavy chain variable domain and a cognate antibody light chain variable domains forming a binding site specifically binding to human TfR comprising
      three heavy chain complementarity determining regions (HCDRs) comprising amino acid sequences of SEQ ID NO: 9 (HCDR1), SEQ ID NO: 10 (HCDR2), and SEQ ID NO: 11 (HCDR3); and
      three light chain complementarity determining regions (LCDRs) comprising amino acid sequences of SEQ ID NO: 13 (LCDR1), SEQ ID NO: 14 (LCDR2), and SEQ ID NO: 15 (LCDR3).
95. The method according to any one of embodiments 90 to 94, wherein the at least one bispecific anti-Abeta/TfR antibody is trontinemab.
96. The method according to any one of embodiments 90 to 95, wherein the therapeutically effective amount of the at least one bispecific anti-Abeta/TfR antibody is 9 mg/kg or less relative to the weight of the subject.
97. The method according to any one of embodiments 90 to 96, wherein the therapeutically effective amount of the at least one bispecific anti-Abeta/TfR antibody is administered once every four weeks.
98. The method according to any one of embodiments 90 to 97, wherein the at least one bispecific anti-Abeta/TfR antibody is administered intravenously.
99. The method according to any one of embodiments 90 to 98, wherein the subject having early Alzheimer's disease has been diagnosed as having mild, mild-to-prodromal or prodromal Alzheimer's disease.
100. The method according to any one of embodiments 90 to 98, wherein the subject having early Alzheimer's disease has been diagnosed as having mild cognitive impairment due to Alzheimer's disease, intermediate likelihood and/or has been diagnosed as having mild Alzheimer's disease dementia.
101. The method according to any one of embodiments 90 to 100, wherein the composition comprises 0.2 mg/kg to 7.2 mg/kg of the at least one anti-Abeta antibody relative to the weight of the subject.
102. The method according to any one of embodiments 90 to 101, wherein the composition comprises 0.6 mg/kg to 7.2 mg/kg of the at least one anti-Abeta antibody relative to the weight of the subject.
103. The method according to any one of embodiments 90 to 102, wherein the composition comprises 1.2 mg/kg to 7.2 mg/kg of the at least one anti-Abeta antibody relative to the weight of the subject.
104. The method according to any one of embodiments 90 to 103, wherein the composition comprises 1.8 mg/kg to 7.2 mg/kg of the at least one anti-Abeta antibody relative to the weight of the subject.
105. The method according to any one of embodiments 90 to 104, wherein the composition comprises 2 mg/kg to 5 mg/kg of the at least one anti-Abeta antibody relative to the weight of the subject.
106. The method according to any one of embodiments 90 to 105, wherein the composition comprises 3 mg/kg to 4 mg/kg of the at least one anti-Abeta antibody relative to the weight of the subject.
107. The method according to any one of embodiments 90 to 101, wherein the composition comprises 0.2 mg/kg to 3.6 mg/kg of the at least one anti-Abeta antibody relative to the weight of the subject.
108. The method according to any one of embodiments 90 to 101 and 107, wherein the composition comprises 0.6 mg/kg to 3.6 mg/kg of the at least one anti-Abeta antibody relative to the weight of the subject.
109. The method according to any one of embodiments 90 to 101 and 107 to 108, wherein the composition comprises 1.2 mg/kg to 3.6 mg/kg of the at least one anti-Abeta antibody relative to the weight of the subject.
110. The method according to any one of embodiments 90 to 109, wherein the composition comprises 1.8 mg/kg to 3.6 mg/kg of the at least one anti-Abeta antibody relative to the weight of the subject.
111. The method according to any one of embodiments 90 to 110, wherein the composition comprises about 1.8 mg/kg or about 1.9 mg/kg or about 2.0 mg/kg or about 2.1 mg/kg or about 2.2 mg/kg or about 2.3 mg/kg or about 2.4 mg/kg or about 2.5 mg/kg or about 2.6 mg/kg or about 2.7 mg/kg or about 2.8 mg/kg or about 2.9 mg/kg or about 3.0 mg/kg or about 3.1 mg/kg or about 3.2 mg/kg or about 3.3 mg/kg or about 3.4 mg/kg or about 3.5 mg/kg or about 3.6 mg/kg of the at least one anti-Abeta antibody relative to the weight of the subject.
112. The method according to any one of embodiments 90 to 111, wherein the composition comprises 1.2 mg/kg to 7.2 mg/kg of the at least one anti-Abeta antibody relative to the weight of the subject for a number of administrations and thereafter the composition comprises 0.2 mg/kg to 3.6 mg/kg of the at least one anti-Abeta antibody relative to the weight of the subject.
113. The method according to embodiment 112, wherein the number of administrations is 3 to 60.
114. The method according to any one of embodiments 90 to 113, wherein the amyloid level of the subject is reduced by at least 20 Centiloids relative to placebo as determined by visual reads of amyloid PET images.
115. The method according to any one of embodiments 90 to 114, wherein the amyloid level of the subject is reduced by at least 25 Centiloids relative to placebo as determined by visual reads of amyloid PET images.
116. The method according to any one of embodiments 90 to 115, wherein the amyloid level of the subject is reduced by at least 60 Centiloids relative to placebo as determined by visual reads of amyloid PET images.
117. The method according to any one of embodiments 90 to 116, wherein the amyloid level of the subject is reduced by at least 75 Centiloids relative to placebo as determined by visual reads of amyloid PET images.
118. The method according to any one of embodiments 90 to 117, wherein the amyloid level of the subject is reduced by at least 80 Centiloids relative to baseline as determined by visual reads of amyloid PET images.
119. The method according to any one of embodiments 90 to 118, wherein the amyloid level of the subject is reduced by at least 85 Centiloids relative to placebo as determined by visual reads of amyloid PET images.
120. The method according to any one of embodiments 90 to 119, wherein the amyloid level of the subject is reduced by at least 90 Centiloids relative to placebo as determined by visual reads of amyloid PET images.
121. The method according to any one of embodiments 90 to 113, wherein the amyloid level of the subject is reduced by at least 20 Centiloids relative to baseline as determined by visual reads of amyloid PET images.
122. The method according to any one of embodiments 90 to 113 and 121, wherein the amyloid level of the subject is reduced by at least 25 Centiloids relative to baseline as determined by visual reads of amyloid PET images.
123. The method according to any one of embodiments 90 to 113 and 121 to 122, wherein the amyloid level of the subject is reduced by at least 60 Centiloids relative to baseline as determined by visual reads of amyloid PET images.
124. The method according to any one of embodiments 90 to 113 and 121 to 123, wherein the amyloid level of the subject is reduced by at least 75 Centiloids relative to baseline as determined by visual reads of amyloid PET images.
125. The method according to any one of embodiments 90 to 113 and 121 to 124, wherein the amyloid level of the subject is reduced by at least 80 Centiloids relative to baseline as determined by visual reads of amyloid PET images.
126. The method according to any one of embodiments 90 to 113 and 121 to 125, wherein the amyloid level of the subject is reduced by at least 85 Centiloids relative to baseline as determined by visual reads of amyloid PET images.
127. The method according to any one of embodiments 90 to 113 and 121 to 126, wherein the amyloid level of the subject is reduced by at least 90 Centiloids relative to baseline as determined by visual reads of amyloid PET images.
128. The method according to any one of embodiments 90 to 127, wherein the reduction is after 18 months of administration of the composition.
129. The method according to any one of embodiments 90 to 128, wherein the reduction is after 12 months of administration of the composition.
130. The method according to any one of embodiments 90 to 129, wherein the reduction is after 6 months of administration of the composition.
131. The method according to any one of embodiments 90 to 130, wherein said administration results in a reduction of cerebrospinal fluid level of Abeta1-42 protein fragment.
132. The method according to any one of embodiments 90 to 131, wherein the subject is concomitantly administered at least one Alzheimer's disease medication other than trontinemab.
133. The method according to any one of embodiments 90 to 131, wherein the subject is not concomitantly administered at least one Alzheimer's disease medication other than trontinemab.
134. A method of treating a subject having early Alzheimer's disease comprising administering a composition comprising a therapeutically effective amount of at least one bispecific anti-Abeta/TfR antibody, wherein the severity of at least one symptom associated with Alzheimer's disease is reduced by at least 10%, 20%, 30%, 40%, 50%, 60%, 70%, 80%, 90%, or 95% relative to the severity of the same symptom in subjects that received placebo.
135. The method of embodiment 134, wherein the severity of at least one symptom associated with Alzheimer's disease is determined by CDR-SB or PET.
136. The method according to any one of embodiments 134 to 135, wherein severity of at least one symptom associated with Alzheimer's disease is reduced by at least 25% relative to placebo as determined by CDR-SB or at least 40% relative to placebo as determined by PET imaging after 18 months of administration of the composition.
137. The method according to any one of embodiments 134 to 137, wherein the severity of at least one symptom associated with Alzheimer's disease is reduced by at least 60% relative to placebo as determined by CDR-SB or at least 75% relative to placebo as determined by PET imaging after 18 months of administration of the composition.
138. The method according to any one of embodiments 134 to 137, wherein the at least one symptom associated with Alzheimer's disease is chosen from clinical decline and brain amyloid level.
139. The method according to any one of embodiments 134 to 138, wherein the at least one bispecific anti-Abeta/TfR antibody comprises
   i) two copies of a first pair of an antibody heavy chain variable domain and a cognate antibody light chain variable domains forming a binding site specifically binding to human Abeta protein each comprising
      three heavy chain complementarity determining regions (HCDRs) comprising amino acid sequences of SEQ ID NO: 1 (HCDR1), SEQ ID NO: 2 (HCDR2), and SEQ ID NO: 3 (HCDR3); and
      three light chain complementarity determining regions (LCDRs) comprising amino acid sequences of SEQ ID NO: 5 (LCDR1), SEQ ID NO: 6 (LCDR2), and SEQ ID NO: 7 (LCDR3); and
   ii) one copy of a second pair of an antibody heavy chain variable domain and a cognate antibody light chain variable domains forming a binding site specifically binding to human TfR comprising
      three heavy chain complementarity determining regions (HCDRs) comprising amino acid sequences of SEQ ID NO: 9 (HCDR1), SEQ ID NO: 10 (HCDR2), and SEQ ID NO: 11 (HCDR3); and
      three light chain complementarity determining regions (LCDRs) comprising amino acid sequences of SEQ ID NO: 13 (LCDR1), SEQ ID NO: 14 (LCDR2), and SEQ ID NO: 15 (LCDR3).
140. The method according to any one of embodiments 134 to 139, wherein the at least one bispecific anti-Abeta/TfR antibody is trontinemab.
141. The method according to any one of embodiments 134 to 140, wherein the therapeutically effective amount of the at least one bispecific anti-Abeta/TfR antibody is 9 mg/kg or less relative to the weight of the subject.
142. The method according to any one of embodiments 134 to 141, wherein the therapeutically effective amount of the at least one bispecific anti-Abeta/TfR antibody is administered once every four weeks.
143. The method according to any one of embodiments 134 to 142, wherein the at least one bispecific anti-Abeta/TfR antibody is administered intravenously.
144. The method according to any one of embodiments 134 to 143, wherein the subject having early Alzheimer's disease has been diagnosed as having mild mild-to-prodromal or prodromal Alzheimer's disease.
145. The method according to any one of embodiments 134 to 144, wherein the subject having early Alzheimer's disease has been diagnosed as having mild cognitive impairment due to Alzheimer's disease, intermediate likelihood and/or has been diagnosed as having mild Alzheimer's disease dementia.
146. The method according to any one of embodiments 134 to 145, wherein the composition comprises 0.2 mg/kg to 7.2 mg/kg of the at least one anti-Abeta antibody relative to the weight of the subject.
147. The method according to any one of embodiments 134 to 146, wherein the composition comprises 0.6 mg/kg to 7.2 mg/kg of the at least one anti-Abeta antibody relative to the weight of the subject.
148. The method according to any one of embodiments 134 to 147, wherein the composition comprises 1.2 mg/kg to 7.2 mg/kg of the at least one anti-Abeta antibody relative to the weight of the subject.
149. The method according to any one of embodiments 134 to 148, wherein the composition comprises 1.8 mg/kg to 7.2 mg/kg of the at least one anti-Abeta antibody relative to the weight of the subject.
150. The method according to any one of embodiments 134 to 149, wherein the composition comprises 2 mg/kg to 5 mg/kg of the at least one anti-Abeta antibody relative to the weight of the subject.
151. The method according to any one of embodiments 134 to 150, wherein the composition comprises 3 mg/kg to 4 mg/kg of the at least one anti-Abeta antibody relative to the weight of the subject.
152. The method according to any one of embodiments 134 to 146, wherein the composition comprises 0.2 mg/kg to 3.6 mg/kg of the at least one anti-Abeta antibody relative to the weight of the subject.
153. The method according to any one of embodiments 134 to 146 and 152, wherein the composition comprises 0.6 mg/kg to 3.6 mg/kg of the at least one anti-Abeta antibody relative to the weight of the subject.
154. The method according to any one of embodiments 134 to 146 and 152 to 153, wherein the composition comprises 1.2 mg/kg to 3.6 mg/kg of the at least one anti-Abeta antibody relative to the weight of the subject.
155. The method according to any one of embodiments 134 to 154, wherein the composition comprises 1.8 mg/kg to 3.6 mg/kg of the at least one anti-Abeta antibody relative to the weight of the subject.
156. The method according to any one of embodiments 134 to 155, wherein the composition comprises about 1.8 mg/kg or about 1.9 mg/kg or about 2.0 mg/kg or about 2.1 mg/kg or about 2.2 mg/kg or about 2.3 mg/kg or about 2.4 mg/kg or about 2.5 mg/kg or about 2.6 mg/kg or about 2.7 mg/kg or about 2.8 mg/kg or about 2.9 mg/kg or about 3.0 mg/kg or about 3.1 mg/kg or about 3.2 mg/kg or about 3.3 mg/kg or about 3.4 mg/kg or about 3.5 mg/kg or about 3.6 mg/kg of the at least one anti-Abeta antibody relative to the weight of the subject.
157. The method according to any one of embodiments 134 to 156, wherein the composition comprises 1.2 mg/kg to 7.2 mg/kg of the at least one anti-Abeta antibody relative to the weight of the subject for a number of administrations and thereafter the composition comprises 0.2 mg/kg to 3.6 mg/kg of the at least one anti-Abeta antibody relative to the weight of the subject.
158. The method according to embodiment 157, wherein the number of administrations is 3 to 60.
159. The method according to any one of embodiments 134 to 158, wherein the amyloid level is reduced by at least 20 Centiloids relative to placebo as determined by visual reads of amyloid PET images.
160. The method according to any one of embodiments 134 to 159, wherein the amyloid level is reduced by at least 25 Centiloids relative to placebo as determined by visual reads of amyloid PET images.
161. The method according to any one of embodiments 134 to 160, wherein the amyloid level is reduced by at least 60 Centiloids relative to placebo as determined by visual reads of amyloid PET images.
162. The method according to any one of embodiments 134 to 161, wherein the amyloid level is reduced by at least 75 Centiloids relative to placebo as determined by visual reads of amyloid PET images.
163. The method according to any one of embodiments 134 to 162, wherein the amyloid level is reduced by at least 80 Centiloids relative to baseline as determined by visual reads of amyloid PET images.
164. The method according to any one of embodiments 134 to 163, wherein the amyloid level is reduced by at least 85 Centiloids relative to placebo as determined by visual reads of amyloid PET images.
165. The method according to any one of embodiments 134 to 164, wherein the amyloid level is reduced by at least 90 Centiloids relative to placebo as determined by visual reads of amyloid PET images.
166. The method according to any one of embodiments 134 to 158, wherein the amyloid level is reduced by at least 20 Centiloids relative to baseline as determined by visual reads of amyloid PET images.
167. The method according to any one of embodiments 134 to 158 and 166, wherein the amyloid level is reduced by at least 25 Centiloids relative to baseline as determined by visual reads of amyloid PET images.
168. The method according to any one of embodiments 134 to 158 and 166 to 167, wherein the amyloid level is reduced by at least 60 Centiloids relative to baseline as determined by visual reads of amyloid PET images.
169. The method according to any one of embodiments 134 to 158 and 166 to 168, wherein the amyloid level is reduced by at least 75 Centiloids relative to baseline as determined by visual reads of amyloid PET images.
170. The method according to any one of embodiments 134 to 158 and 166 to 169, wherein the amyloid level is reduced by at least 80 Centiloids relative to baseline as determined by visual reads of amyloid PET images.
171. The method according to any one of embodiments 134 to 158 and 166 to 170, wherein the amyloid level is reduced by at least 85 Centiloids relative to baseline as determined by visual reads of amyloid PET images.
172. The method according to any one of embodiments 134 to 158 and 166 to 171, wherein the amyloid level is reduced by at least 90 Centiloids relative to baseline as determined by visual reads of amyloid PET images.
173. The method according to any one of embodiments 134 to 172, wherein the reduction of the amyloid level is after 18 months of administration of the composition.
174. The method according to any one of embodiments 134 to 173, wherein the reduction of the amyloid level is after 12 months of administration of the composition.
175. The method according to any one of embodiments 134 to 174, wherein the reduction of the amyloid level is after 6 months of administration of the composition.
176. The method according to any one of embodiments 134 to 175, wherein said administration results in a reduction of cerebrospinal fluid level of Abeta1-42 protein fragment.
177. The method according to any one of embodiments 134 to 176, wherein the subject is concomitantly administered at least one Alzheimer's disease medication other than trontinemab.
178. The method according to any one of embodiments 134 to 177, wherein the subject is not concomitantly administered at least one Alzheimer's disease medication other than trontinemab.
179. A bispecific anti-Abeta/TfR antibody for use as a medicament in the treatment of Alzheimer's Disease (in a subject).
180. A bispecific anti-Abeta/TfR antibody for use in the treatment of Alzheimer's Disease (in a subject).
181. The bispecific anti-Abeta/TfR antibody for use according to any one of embodiments 179 to 180, wherein administration of the antibody results in at least 30% of subjects being amyloid negative as determined by visual reads of amyloid PET images after 3 months of administration of the composition.
182. The bispecific anti-Abeta/TfR antibody for use according to any one of embodiments 179 to 181, wherein administration of the antibody results in at least 50% of the subjects being amyloid negative, as determined by visual reads of amyloid PET images, after 12 months of administration of the composition.
183. The bispecific anti-Abeta/TfR antibody for use according to any one of embodiments 179 to 182 wherein administration of the antibody results in at least 70% of the subjects being amyloid negative, as determined by visual reads of amyloid PET images, after 18 months of administration of the composition.
184. The bispecific anti-Abeta/TfR antibody for use according to any one of embodiments 179 to 183, wherein the bispecific anti-Abeta/TfR antibody comprises
   i) two copies of a first pair of an antibody heavy chain variable domain and a cognate antibody light chain variable domains forming a binding site specifically binding to human Abeta protein each comprising
      three heavy chain complementarity determining regions (HCDRs) comprising amino acid sequences of SEQ ID NO: 1 (HCDR1), SEQ ID NO: 2 (HCDR2), and SEQ ID NO: 3 (HCDR3); and
      three light chain complementarity determining regions (LCDRs) comprising amino acid sequences of SEQ ID NO: 5 (LCDR1), SEQ ID NO: 6 (LCDR2), and SEQ ID NO: 7 (LCDR3); and
   ii) one copy of a second pair of an antibody heavy chain variable domain and a cognate antibody light chain variable domains forming a binding site specifically binding to human TfR comprising
      three heavy chain complementarity determining regions (HCDRs) comprising amino acid sequences of SEQ ID NO: 9 (HCDR1), SEQ ID NO: 10 (HCDR2), and SEQ ID NO: 11 (HCDR3); and
      three light chain complementarity determining regions (LCDRs) comprising amino acid sequences of SEQ ID NO: 13 (LCDR1), SEQ ID NO: 14 (LCDR2), and SEQ ID NO: 15 (LCDR3).
185. The bispecific anti-Abeta/TfR antibody for use according to any one of embodiments 179 to 184, wherein the bispecific anti-Abeta/TfR antibody is trontinemab.
186. The bispecific anti-Abeta/TfR antibody for use according to any one of embodiments 179 to 185, wherein the antibody is administered in a therapeutically effective amount.
187. The bispecific anti-Abeta/TfR antibody for use according to any one of embodiments 179 to 186, wherein the antibody is administered at a dose of 9 mg/kg or less (relative to the weight of the subject).
188. The bispecific anti-Abeta/TfR antibody for use according to any one of embodiments 179 to 187, wherein the antibody is administered once every four weeks.
189. The bispecific anti-Abeta/TfR antibody for use according to any one of embodiments 179 to 188, wherein the antibody is administered intravenously.
190. The bispecific anti-Abeta/TfR antibody for use according to any one of embodiments 179 to 189, wherein the treatment is of a subject having early Alzheimer's disease.
191. The bispecific anti-Abeta/TfR antibody for use according to any one of embodiments 179 to 190, wherein the treatment is of a subject that has been diagnosed as having mild, mild-to-prodromal or prodromal Alzheimer's disease.
192. The bispecific anti-Abeta/TfR antibody for use according to any one of embodiments 179 to 191, wherein the treatment is of a subject having early Alzheimer's disease that has been diagnosed as having mild cognitive impairment due to Alzheimer's disease, intermediate likelihood and/or has been diagnosed as having mild Alzheimer's disease dementia.
193. The bispecific anti-Abeta/TfR antibody for use according to any one of embodiments 179 to 192, wherein the composition comprises 0.2 mg/kg to 7.2 mg/kg of the at least one anti-Abeta antibody relative to the weight of the subject.
194. The bispecific anti-Abeta/TfR antibody for use according to any one of embodiments 179 to 193, wherein the composition comprises 0.6 mg/kg to 7.2 mg/kg of the at least one anti-Abeta antibody relative to the weight of the subject.
195. The bispecific anti-Abeta/TfR antibody for use according to any one of embodiments 179 to 194, wherein the composition comprises 1.2 mg/kg to 7.2 mg/kg of the at least one anti-Abeta antibody relative to the weight of the subject.
196. The bispecific anti-Abeta/TfR antibody for use according to any one of embodiments 179 to 195, wherein the composition comprises 1.8 mg/kg to 7.2 mg/kg of the at least one anti-Abeta antibody relative to the weight of the subject.
197. The bispecific anti-Abeta/TfR antibody for use according to any one of embodiments 179 to 196, wherein the composition comprises 2 mg/kg to 5 mg/kg of the at least one anti-Abeta antibody relative to the weight of the subject.
198. The bispecific anti-Abeta/TfR antibody for use according to any one of embodiments 179 to 197, wherein the composition comprises 3 mg/kg to 4 mg/kg of the at least one anti-Abeta antibody relative to the weight of the subject.
199. The bispecific anti-Abeta/TfR antibody for use according to any one of embodiments 179 to 193, wherein the composition comprises 0.2 mg/kg to 3.6 mg/kg of the at least one anti-Abeta antibody relative to the weight of the subject.
200. The bispecific anti-Abeta/TfR antibody for use according to any one of embodiments 179 to 193 and 199, wherein the composition comprises 0.6 mg/kg to 3.6 mg/kg of the at least one anti-Abeta antibody relative to the weight of the subject.
201. The bispecific anti-Abeta/TfR antibody for use according to any one of embodiments 179 to 193 and 199 to 201, wherein the composition comprises 1.2 mg/kg to 3.6 mg/kg of the at least one anti-Abeta antibody relative to the weight of the subject.
202. The bispecific anti-Abeta/TfR antibody for use according to any one of embodiments 179 to 201, wherein the composition comprises 1.8 mg/kg to 3.6 mg/kg of the at least one anti-Abeta antibody relative to the weight of the subject.
203. The bispecific anti-Abeta/TfR antibody for use according to any one of embodiments 179 to 202, wherein the composition comprises about 1.8 mg/kg or about 1.9 mg/kg or about 2.0 mg/kg or about 2.1 mg/kg or about 2.2 mg/kg or about 2.3 mg/kg or about 2.4 mg/kg or about 2.5 mg/kg or about 2.6 mg/kg or about 2.7 mg/kg or about 2.8 mg/kg or about 2.9 mg/kg or about 3.0 mg/kg or about 3.1 mg/kg or about 3.2 mg/kg or about 3.3 mg/kg or about 3.4 mg/kg or about 3.5 mg/kg or about 3.6 mg/kg of the at least one anti-Abeta antibody relative to the weight of the subject.
204. The bispecific anti-Abeta/TfR antibody for use according to any one of embodiments 179 to 203, wherein the composition comprises 1.2 mg/kg to 7.2 mg/kg of the at least one anti-Abeta antibody relative to the weight of the subject for a number of administrations and thereafter the composition comprises 0.2 mg/kg to 3.6 mg/kg of the at least one anti-Abeta antibody relative to the weight of the subject.
205. The bispecific anti-Abeta/TfR antibody for use according to embodiment 204, wherein the number of administrations is 3 to 60.
206. The bispecific anti-Abeta/TfR antibody for use according to any one of embodiments 179 to 205, wherein the administration of the antibody reduces the amyloid level by at least 75 Centiloids relative to placebo as determined by visual reads of amyloid PET images.
207. The bispecific anti-Abeta/TfR antibody for use according to any one of embodiments 179 to 206, wherein the administration of the antibody reduces the amyloid level by at least 80 Centiloids relative to baseline as determined by visual reads of amyloid PET images.
208. The bispecific anti-Abeta/TfR antibody for use according to any one of embodiments 179 to 207, wherein the administration of the antibody reduces the amyloid level by at least 85 Centiloids relative to placebo as determined by visual reads of amyloid PET images.
209. The bispecific anti-Abeta/TfR antibody for use according to any one of embodiments 179 to 208, wherein the administration of the antibody reduces the amyloid level by at least 90 Centiloids relative to placebo as determined by visual reads of amyloid PET images.
210. The bispecific anti-Abeta/TfR antibody for use according to any one of embodiments 179 to 205, wherein the administration of the antibody reduces the amyloid level by at least 75 Centiloids relative to baseline as determined by visual reads of amyloid PET images.
211. The bispecific anti-Abeta/TfR antibody for use according to any one of embodiments 179 to 205 and 210, wherein the administration of the antibody reduces the amyloid level by at least 80 Centiloids relative to baseline as determined by visual reads of amyloid PET images.
212. The bispecific anti-Abeta/TfR antibody for use according to any one of embodiments 179 to 205 and 210 to 211, wherein the administration of the antibody reduces the amyloid level by at least 85 Centiloids relative to baseline as determined by visual reads of amyloid PET images.
213. The bispecific anti-Abeta/TfR antibody for use according to any one of embodiments 179 to 205 and 210 to 212, wherein the administration of the antibody reduces the amyloid level by at least 90 Centiloids relative to baseline as determined by visual reads of amyloid PET images.
214. The bispecific anti-Abeta/TfR antibody for use according to any one of embodiments 179 to 213, wherein the administration of the antibody reduces the amyloid level within 18 months after the start of the administration of the antibody.
215. The bispecific anti-Abeta/TfR antibody for use according to any one of embodiments 179 to 214, wherein the administration of the antibody reduces the amyloid level within 12 months after the start of the administration of the antibody.
216. The bispecific anti-Abeta/TfR antibody for use according to any one of embodiments 179 to 215, wherein the administration of the antibody reduces the amyloid level within 6 months after the start of the administration of the antibody.
217. The bispecific anti-Abeta/TfR antibody for use according to any one of embodiments 179 to 216, wherein the administration of the antibody reduces the amyloid level within 3 months after the start of the administration of the antibody.
218. The bispecific anti-Abeta/TfR antibody for use according to any one of embodiments 179 to 217, wherein the administration of the antibody results in a reduction of cerebrospinal fluid level of Abeta1-42 protein fragment.
219. The bispecific anti-Abeta/TfR antibody for use according to any one of embodiments 179 to 218, wherein at least one Alzheimer's disease medication other than trontinemab is administered.
220. The bispecific anti-Abeta/TfR antibody for use according to any one of embodiments 179 to 219, wherein no further Alzheimer's disease medication other than trontinemab is administered.
221. Use of a bispecific anti-Abeta/TfR antibody for the treatment of Alzheimer's Disease.
222. Use of a bispecific anti-Abeta/TfR antibody in a method of treating Alzheimer's disease.
223. The use according to any one of embodiments 221 to 222, wherein the use of the antibody results in at least 30% of subjects being amyloid negative as determined by visual reads of amyloid PET images after 3 months of administration of the composition.
224. The use according to any one of embodiments 221 to 223, wherein the use of the antibody results in at least 50% of the subjects being amyloid negative, as determined by visual reads of amyloid PET images, after 12 months of administration of the composition.
225. The use according to any one of embodiments 221 to 224 wherein the use of the antibody results in at least 70% of the subjects being amyloid negative, as determined by visual reads of amyloid PET images, after 18 months of administration of the composition.
226. The use according to any one of embodiments 221 to 225, wherein the bispecific anti-Abeta/TfR antibody comprises
   i) two copies of a first pair of an antibody heavy chain variable domain and a cognate antibody light chain variable domains forming a binding site specifically binding to human Abeta protein each comprising
      three heavy chain complementarity determining regions (HCDRs) comprising amino acid sequences of SEQ ID NO: 1 (HCDR1), SEQ ID NO: 2 (HCDR2), and SEQ ID NO: 3 (HCDR3); and
      three light chain complementarity determining regions (LCDRs) comprising amino acid sequences of SEQ ID NO: 5 (LCDR1), SEQ ID NO: 6 (LCDR2), and SEQ ID NO: 7 (LCDR3); and
   ii) one copy of a second pair of an antibody heavy chain variable domain and a cognate antibody light chain variable domains forming a binding site specifically binding to human TfR comprising
      three heavy chain complementarity determining regions (HCDRs) comprising amino acid sequences of SEQ ID NO: 9 (HCDR1), SEQ ID NO: 10 (HCDR2), and SEQ ID NO: 11 (HCDR3); and
      three light chain complementarity determining regions (LCDRs) comprising amino acid sequences of SEQ ID NO: 13 (LCDR1), SEQ ID NO: 14 (LCDR2), and SEQ ID NO: 15 (LCDR3).
227. The use according to any one of embodiments 221 to 226, wherein the bispecific anti-Abeta/TfR antibody is trontinemab.
228. The use according to any one of embodiments 221 to 227, wherein the antibody is used/administered in a therapeutically effective amount.
229. The use according to any one of embodiments 221 to 228, wherein the antibody is used/administered at a dose of 9 mg/kg or less (relative to the weight of the subject).
230. The use according to any one of embodiments 221 to 229, wherein the antibody is used/administered once every four weeks.
231. The use according to any one of embodiments 221 to 230, wherein the antibody is used/administered intravenously.
232. The use according to any one of embodiments 221 to 231, wherein the treatment is of a subject having early Alzheimer's disease.
233. The use according to any one of embodiments 221 to 232, wherein the treatment is of a subject that has been diagnosed as having mild, mild-to-prodromal or prodromal Alzheimer's disease.
234. The use according to any one of embodiments 221 to 233, wherein the treatment is of a subject having early Alzheimer's disease that has been diagnosed as having mild cognitive impairment due to Alzheimer's disease, intermediate likelihood and/or has been diagnosed as having mild Alzheimer's disease dementia.
235. The use according to any one of embodiments 221 to 234, wherein the composition comprises 0.2 mg/kg to 7.2 mg/kg of the at least one anti-Abeta antibody relative to the weight of the subject.
236. The use according to any one of embodiments 221 to 235, wherein the composition comprises 0.6 mg/kg to 7.2 mg/kg of the at least one anti-Abeta antibody relative to the weight of the subject.
237. The use according to any one of embodiments 221 to 236, wherein the composition comprises 1.2 mg/kg to 7.2 mg/kg of the at least one anti-Abeta antibody relative to the weight of the subject.
238. The use according to any one of embodiments 221 to 237, wherein the composition comprises 1.8 mg/kg to 7.2 mg/kg of the at least one anti-Abeta antibody relative to the weight of the subject.
239. The use according to any one of embodiments 221 to 238, wherein the composition comprises 2 mg/kg to 5 mg/kg of the at least one anti-Abeta antibody relative to the weight of the subject.
240. The use according to any one of embodiments 221 to 239, wherein the composition comprises 3 mg/kg to 4 mg/kg of the at least one anti-Abeta antibody relative to the weight of the subject.
241. The use according to any one of embodiments 221 to 235, wherein the composition comprises 0.2 mg/kg to 3.6 mg/kg of the at least one anti-Abeta antibody relative to the weight of the subject.
242. The use according to any one of embodiments 221 to 235 and 241, wherein the composition comprises 0.6 mg/kg to 3.6 mg/kg of the at least one anti-Abeta antibody relative to the weight of the subject.
243. The use according to any one of embodiments 221 to 235 and 241 to 242, wherein the composition comprises 1.2 mg/kg to 3.6 mg/kg of the at least one anti-Abeta antibody relative to the weight of the subject.
244. The use according to any one of embodiments 221 to 243, wherein the composition comprises 1.8 mg/kg to 3.6 mg/kg of the at least one anti-Abeta antibody relative to the weight of the subject.
245. The use according to any one of embodiments 221 to 244, wherein the composition comprises about 1.8 mg/kg or about 1.9 mg/kg or about 2.0 mg/kg or about 2.1 mg/kg or about 2.2 mg/kg or about 2.3 mg/kg or about 2.4 mg/kg or about 2.5 mg/kg or about 2.6 mg/kg or about 2.7 mg/kg or about 2.8 mg/kg or about 2.9 mg/kg or about 3.0 mg/kg or about 3.1 mg/kg or about 3.2 mg/kg or about 3.3 mg/kg or about 3.4 mg/kg or about 3.5 mg/kg or about 3.6 mg/kg of the at least one anti-Abeta antibody relative to the weight of the subject.
246. The use according to any one of embodiments 221 to 245, wherein the composition comprises 1.2 mg/kg to 7.2 mg/kg of the at least one anti-Abeta antibody relative to the weight of the subject for a number of administrations and thereafter the composition comprises 0.2 mg/kg to 3.6 mg/kg of the at least one anti-Abeta antibody relative to the weight of the subject.
247. The use according to embodiment 246, wherein the number of administrations is 3 to 60.
248. The use according to any one of embodiments 221 to 247, wherein the use/administration of the antibody reduces the amyloid level by at least 75 Centiloids relative to placebo as determined by visual reads of amyloid PET images.
249. The use according to any one of embodiments 221 to 248, wherein the use/administration of the antibody reduces the amyloid level by at least 80 Centiloids relative to baseline as determined by visual reads of amyloid PET images.
250. The use according to any one of embodiments 221 to 249, wherein the use/administration of the antibody reduces the amyloid level by at least 85 Centiloids relative to placebo as determined by visual reads of amyloid PET images.
251. The use according to any one of embodiments 221 to 250, wherein the use/administration of the antibody reduces the amyloid level by at least 90 Centiloids relative to placebo as determined by visual reads of amyloid PET images.
252. The use according to any one of embodiments 221 to 247, wherein the use/administration of the antibody reduces the amyloid level by at least 75 Centiloids relative to baseline as determined by visual reads of amyloid PET images.
253. The use according to any one of embodiments 221 to 247 and 252, wherein the use/administration of the antibody reduces the amyloid level by at least 80 Centiloids relative to baseline as determined by visual reads of amyloid PET images.
254. The use according to any one of embodiments 221 to 247 and 252 to 253, wherein the use/administration of the antibody reduces the amyloid level by at least 85 Centiloids relative to baseline as determined by visual reads of amyloid PET images.
255. The use according to any one of embodiments 221 to 247 and 252 to 254, wherein the use/administration of the antibody reduces the amyloid level by at least 90 Centiloids relative to baseline as determined by visual reads of amyloid PET images.
256. The use according to any one of embodiments 221 to 255, wherein the use/administration of the antibody reduces the amyloid level within 18 months after the start of the use/administration of the antibody.
257. The use according to any one of embodiments 221 to 256, wherein the use/administration of the antibody reduces the amyloid level within 12 months after the start of the use/administration of the antibody.
258. The use according to any one of embodiments 221 to 257, wherein the use/administration of the antibody reduces the amyloid level within 6 months after the start of the use/administration of the antibody.
259. The use according to any one of embodiments 221 to 257, wherein the use/administration of the antibody reduces the amyloid level within 3 months after the start of the use/administration of the antibody.
260. The use according to any one of embodiments 221 to 259, wherein the use/administration of the antibody results in a reduction of cerebrospinal fluid level of Abeta1-42 protein fragment.
261. The use according to any one of embodiments 221 to 260, wherein at least one Alzheimer's disease medication other than trontinemab is used/administered.
262. The use according to any one of embodiments 221 to 260, wherein no further Alzheimer's disease medication other than trontinemab is used/administered.
263. The method or the antibody for use or the use according to any one of embodiments 1 to 262, wherein the subject has white skin.
264. The method or the antibody for use of the use according to any one of embodiments 1 to 262, wherein the subject is not a person of color.
265. The method or the antibody for use of the use according to any one of embodiments 1 to 264, wherein the subject has a body weight of 60 to 70 kg.
266. The method or the antibody for use of the use according to any one of embodiments 1 to 265, wherein the subject is an APOE ε4 carrier.

### BRIEF DESCRIPTION OF THE DRAWINGS

FIG. 1: FIG. 1 depicts a sketch of the structure of trontinemab/RG6102.

FIG. 2A: FIG. 2A depicts the result of brain (tissue) exposure in non-human primate Hippocampus with trontinemab compared to gantenerumab after a single IV dose of 10 mg/kg trontinemab versus a single IV dose of 20 mg/kg gantenerumab.

FIG. 2B: FIG. 2B depicts the brain to plasma ratio in non-human primate Cortex, Hippocampus, and Striatum achieved with trontinemab compared to gantenerumab, shown as fold-change in Kp, Cmax and AUC.

FIG. 3: FIG. 3 depicts the course of amyloid depletion in Cohorts 1 to 3 shown as mean amyloid PET burden in Centiloids over 28 weeks.

FIG. 4: FIG. 4 depicts the course of amyloid depletion in Cohorts 1 to 3 shown as mean amyloid reduction from baseline in Centiloids over 28 weeks.

FIG. 5: FIG. 5 depicts the course of amyloid depletion in Cohorts 1 to 4 shown as mean amyloid reduction from baseline in Centiloids over 12 and 28 weeks, respectively.

FIG. 6: FIG. 6 depicts a comparison of trontinemab treatment effects in humans to other anti-Abeta treatment studies (GRADUATE I, GRADUATE II, CLARITY AD, TRAILBLAZER-ALZ-2) shown as amyloid PET in Centiloids over −27 months.

### DETAILED DESCRIPTION

The presently disclosed subject matter relates, in certain embodiments, to methods comprising the administration of bispecific antibodies that specifically bind to human Abeta protein and human transferrin receptor. For example, in certain embodiments, the methods described herein comprise administering such bispecific antibodies at lower dosages and/or at lower frequencies as compared to current anti-Abeta protein therapies known in the art. In certain embodiments, the methods described herein-despite being administered at such lower dosages and/or lower frequencies-achieve greater exposure to the patient's brain tissue and/or more efficient reduction in the patient's Abeta protein plaque as compared to current anti-Abeta protein therapies known in the art. In certain embodiments, the methods described herein result in a lower incidence of ARIA (Amyloid-Related Imaging Abnormalities) as compared to current anti-Abeta protein therapies known in the art. The presently disclosed subject matter further provides compositions comprising such bispecific antibodies at such dosages and or configured for such frequency dosing for performing such methods.

One aspect of the current invention is a method of reducing amyloid level in a subject having mild or prodromal Alzheimer's disease comprising intravenously administering a composition of the bispecific anti-Abeta/TfR antibody trontinemab/RG6102 with a dose in the range of 1.2 mg/kg to 7.2 mg/kg relative to the weight of the subject once every four weeks.

One aspect according to the current invention is a method of reducing amyloid level in a subject having mild or prodromal Alzheimer's disease comprising intravenously administering a composition of the bispecific anti-Abeta/TfR antibody trontinemab/RG6102 with a dose in the range of 1.2 mg/kg to 7.2 mg/kg relative to the weight of the subject once every four weeks.

One aspect according to the current invention is a method of reducing amyloid level in a subject having mild or prodromal Alzheimer's disease comprising intravenously administering a composition of the bispecific anti-Abeta/TfR antibody trontinemab/RG6102 with a dose in the range of 1.2 mg/kg to 7.2 mg/kg relative to the weight of the subject once every four weeks, wherein the administered dose results in a lower number of ARIA events compared to a monospecific anti-Abeta antibody. In certain embodiments of this aspect, the monospecific anti-Abeta antibody is administered at a dose of 10 mg/kg.

One aspect according to the current invention is a method of converting an amyloid positive subject having mild or moderate Alzheimer's disease to amyloid negative subject comprising intravenously administering a composition of the bispecific anti-Abeta/TfR antibody trontinemab/RG6102 with a dose in the range of 1.2 mg/kg to 7.2 mg/kg relative to the weight of the subject once every four weeks. In one preferred embodiment of this aspect, the administration of the composition results in at least 30% of subjects being amyloid negative as determined by visual reads of amyloid PET images after 3 months of administration of a dose of 1.8 mg/kg.

Amyloid-positivity or -negativity is, in one embodiment, determined by visual read of PET scans or Centiloids. In one embodiment the threshold between amyloid-negative and amyloid-positive is 24.1 Centiloids, i.e. amyloid-negative is below 24.1 Centiloids and amyloid positive is at 24.1 or more Centiloids.

One aspect according to the current invention is a method of treating a subject having mild or moderate Alzheimer's disease comprising intravenously administering a composition of the bispecific anti-Abeta/TfR antibody trontinemab/RG6102 with a dose in the range of 1.2 mg/kg to 7.2 mg/kg relative to the weight of the subject once every four weeks, wherein the severity of at least one symptom associated with Alzheimer's disease is reduced by at least 10%, 20%, 30%, 40%, 50%, 60%, 70%, 80%, 90%, or 95% relative to the severity of the same symptom in the same subject prior to treatment. In one preferred embodiment of this aspect, the at least one symptom associated with Alzheimer's disease is brain amyloid level.

One aspect according to the current invention is a method of treating a subject having early Alzheimer's disease comprising intravenously administering a composition of the bispecific anti-Abeta/TfR antibody trontinemab/RG6102 with a dose in the range of 1.2 mg/kg to 7.2 mg/kg relative to the weight of the subject once every four weeks, wherein the severity of at least one symptom associated with Alzheimer's disease is reduced by at least 10%, 20%, 30%, 40%, 50%, 60%, 70%, 80%, 90%, or 95% relative to the severity of the same symptom in subjects that received placebo. In one preferred embodiment of this aspect, the at least one symptom associated with Alzheimer's disease is brain amyloid level.

One aspect according to the current invention is the bispecific anti-Abeta/TfR antibody trontinemab/RG6102 for use as a medicament in the treatment of Alzheimer's Disease, wherein the antibody is intravenously administered with a dose in the range of 1.2 mg/kg to 7.2 mg/kg relative to the weight of the subject once every four weeks. In one preferred embodiment of this aspect, the administration of the composition results in at least 30% of subjects being amyloid negative as determined by visual reads of amyloid PET images after 3 months of administration of a dose of 1.8 mg/kg.

One aspect according to the current invention is a bispecific anti-Abeta/TfR antibody for use in the treatment of Alzheimer's Disease, wherein the antibody is intravenously administered with a dose in the range of 1.2 mg/kg to 7.2 mg/kg relative to the weight of the subject once every four weeks. In one preferred embodiment of this aspect, the administration of the composition results in at least 30% of subjects being amyloid negative as determined by visual reads of amyloid PET images after 3 months of administration of a dose of 1.8 mg/kg. In one preferred embodiment of this aspect, the administration of the composition results in at least 70% of subjects being amyloid negative as determined by visual reads of amyloid PET images after 3 to 6 months of administration of a dose of 1.8 mg/kg (3 to 6 doses).

One aspect of the current invention is the use of the bispecific anti-Abeta/TfR antibody trontinemab/RG6102 for the treatment of Alzheimer's Disease, wherein the antibody is intravenously administered with a dose in the range of 1.2 mg/kg to 7.2 mg/kg relative to the weight of the subject once every four weeks. In one preferred embodiment of this aspect, the administration of the composition results in at least 30% of subjects being amyloid negative as determined by visual reads of amyloid PET images after 3 months of administration of a dose of 1.8 mg/kg. In one preferred embodiment of this aspect, the administration of the composition results in at least 70% of subjects being amyloid negative as determined by visual reads of amyloid PET images after 3 to 6 months of administration of a dose of 1.8 mg/kg (3 to 6 doses).

One aspect of the current invention is the use of the bispecific anti-Abeta/TfR antibody trontinemab/RG6102 in a method of treating Alzheimer's disease, wherein the antibody is intravenously administered with a dose in the range of 1.2 mg/kg to 7.2 mg/kg relative to the weight of the subject once every four weeks. In one preferred embodiment of this aspect, the administration of the composition results in at least 30% of subjects being amyloid negative as determined by visual reads of amyloid PET images after 3 months of administration of a dose of 1.8 mg/kg. In one preferred embodiment of this aspect, the administration of the composition results in at least 70% of subjects being amyloid negative as determined by visual reads of amyloid PET images after 3 to 6 months of administration of a dose of 1.8 mg/kg (3 to 6 doses).

In one preferred embodiment of all aspects and embodiments of the current invention, the dose is in the range of 1.8 mg/kg to 3.6 mg/kg of the antibody relative to the weight of the subject.

In one preferred embodiment of all aspects and embodiments of the current invention, the amyloid level is reduced by at least 20 Centiloids relative to baseline as determined by visual reads of amyloid PET images after 3 months/3 administrations.

In one preferred embodiment of all aspects and embodiments of the current invention, the amyloid level is reduced by at least 25 Centiloids relative to baseline as determined by visual reads of amyloid PET images after 3 months/3 administrations.

In one preferred embodiment of all aspects and embodiments of the current invention, the amyloid level is reduced by at least 60 Centiloids relative to baseline as determined by visual reads of amyloid PET images after 3 months/3 administrations.

While not wishing to be bound by theory, the initial removal of amyloid as determined by PET is expected to only be the “tip of iceberg” with respect to the presently disclosed administration of bispecific anti-Abeta/TfR antibodies, such as, e.g., Trontinemab. As soluble oligomeric species are not measurable using PET, and removing insoluble amyloid does not remove the underlying driver to re-accumulation, the administration of bispecific anti-Abeta/TfR antibodies, such as, e.g., Trontinemab, according to the current invention encompasses sustained suppression of amyloid as an option rather than waiting until substantial pathology re-accumulates.

In certain embodiments, the present invention is directed to a bispecific anti-Abeta/TfR antibody for use in the treatment of Alzheimer's Disease, wherein the antibody is intravenously administered with a dose in the range of 1.2 mg/kg to 7.2 mg/kg relative to the weight of the subject for an initial period and thereafter administering a maintenance dose. In certain embodiments the maintenance dose is a reduced dose relative to the dose administered during the initial period. For example, but not by way of limitation, such maintenance dose can be in the range of 1.8 mg/kg to 3.6 mg/kg of the antibody relative to the weight of the subject. In certain embodiments the maintenance dose is 1.8 mg/kg of the antibody relative to the weight of the subject. In certain embodiments, the maintenance dose is 3.6 mg/kg of the antibody relative to the weight of the subject.

Drug labelling is also referred to as prescription labelling, is a written, printed or graphic matter upon any drugs or any of its container, or accompanying such a drug. Drug labels seek to identify drug contents and to state specific instructions or warnings for administration, storage and disposal. The approved drug labeling for healthcare providers gives key information about the drug that includes:
The specific diseases and conditions that the drug is approved to treat.
How to use the drug to treat those specific diseases and conditions.
Information about the risks of the drug.
Information that healthcare providers should discuss with patients before they take a drug.
Exemplary prescription labelling stating the use of the bispecific anti-Abeta/TfR antibodies of the present invention, such as, e.g., Trontinemab, can comprise: Trontinemab is an amyloid beta-directed antibody indicated for the treatment of Alzheimer's disease. Treatment with trontinemab should be initiated in patients with mild cognitive impairment or mild dementia stage of disease, the population in which treatment was initiated in clinical trials. There are no safety or effectiveness data on initiating treatment at earlier or later stages of the disease than were studied.
Confirm the presence of amyloid beta pathology prior to initiating treatment.
Obtain a recent baseline brain MRI prior to initiating treatment.
Obtain MRIs prior to the 5th, 7th, 9th, and 12th infusions; if radiographically observed ARIA occurs, treatment recommendations are based on type, severity, and presence of symptoms.
Dilution in 0.9% Sodium Chloride Injection, USP, is required prior to administration.
Administer as an intravenous infusion over approximately one hour via a terminal low-protein binding 0.2 or 0.22 micron in-line filter.

In preferred embodiments of all aspects and embodiments, the methods and compositions according to the current invention comprising a bispecific anti-Abeta/TfR antibody or trontinemab are for one or more of the following:
for the treatment of patients with mild cognitive impairment or mild dementia stage of disease or for the treatment of patients with mild, mild-to-moderate or moderate Alzheimer's disease; or/and
for the treatment of patients wherein the presence of amyloid beta pathology prior to initiating treatment has been confirmed; or/and
for the treatment of patients whereof a recent baseline brain MRI prior to initiating treatment has been obtained; or/and
for the treatment of patients whereof MRIs are obtained prior to the 5^{th} or/and 7^{th} or/and 9^{th} or/and 12^{th} infusions has been obtained and if radiographically observed ARIA occurs, treatment recommendations are based on type, severity, and presence of symptoms; or/and
for the treatment the antibody solution is diluted in 0.9% Sodium Chloride Injection, USP, prior to administration; or/and
for the treatment the antibody solution is administered as an intravenous infusion over approximately one hour via a terminal low-protein binding 0.2 or 0.22 micron in-line filter.

### Definitions

It must be noted that as used herein and in the appended claims, the singular forms “a”, “an”, and “the” include plural reference unless the context clearly dictates otherwise. Thus, for example, reference to “a cell” includes a plurality of such cells and equivalents thereof known to those skilled in the art, and so forth. As well, the terms “a” (or “an”), “one or more” and “at least one” can be used interchangeably herein. It is also to be noted that the terms “comprising”, “including”, and “having” can be used interchangeably.

The term “about” denotes a range of +/−20% of the thereafter following numerical value. In one embodiment the term about denotes a range of +/−10% of the thereafter following numerical value. In one embodiment the term about denotes a range of +/−5% of the thereafter following numerical value.

The term “comprising” also includes the term “consisting of”.

The term “anti-(human)Abeta protein antibody” or “anti-Abeta antibody” refers to an antibody that is capable of binding the human Abeta protein with sufficient affinity such that the antibody is useful as a diagnostic and/or therapeutic agent in targeting the Abeta protein. It is of note that the human Abeta protein has several naturally occurring forms, whereby the human forms are referred to as Aβ39, Aβ40, Aβ41, Aβ42 and Aβ43. The most prominent form,

Aβ42, has the amino acid sequence of SEQ ID NO: 45. In Aβ41, Aβ40, Aβ39, the C-terminal amino acids A, IA and VIA are missing, respectively. In the Aβ43 form, an additional threonine residue is comprised at the C-terminus of SEQ ID NO: 45. In one preferred embodiment, the antibody according to the invention specifically binds to the human Abeta protein that has the amino acid sequence of SEQ ID NO: 45.

The “central nervous system” or “CNS” refers to the complex of nerve tissues that control bodily function and includes the brain and spinal cord.

“Affinity” refers to the strength of the sum of all non-covalent interactions between a single binding site of a molecule (e.g., an antibody) and its binding partner (e.g., an antigen). Unless indicated otherwise, as used herein, “binding affinity” refers to intrinsic binding affinity that reflects a 1:1 interaction between members of a binding pair (e.g., antibody and antigen). Affinity can be measured by common methods known in the art, including those described herein.

The term “antibody-dependent cellular cytotoxicity (ADCC)” is a function mediated by Fc receptor binding and refers to lysis of target cells by an antibody as reported herein in the presence of effector cells. ADCC can be measured by the treatment of a preparation of CD19 expressing erythroid cells (e.g., K562 cells expressing recombinant human CD19) with an antibody according to the current invention in the presence of effector cells such as freshly isolated PBMC (peripheral blood mononuclear cells) or purified effector cells from buffy coats, like monocytes or NK (natural killer) cells. Target cells are labeled with 51Cr and subsequently incubated with the antibody. The labeled cells are incubated with effector cells and the supernatant is analyzed for released 51Cr. Controls include the incubation of the target endothelial cells with effector cells but without the antibody. The capacity of the antibody to induce the initial steps mediating ADCC is investigated by measuring their binding to Fcγ receptors expressing cells, such as cells, recombinantly expressing FcγRI and/or FcγRIIA or NK cells (expressing essentially FcγRIIIA).

The term “binding (to an antigen)” denotes the binding of an antibody to its cognate antigen. Binding can be determined in an in vitro assay. In certain embodiments, binding is determined in a binding assay in which the antibody is bound to a surface and binding of the antigen to the antibody is measured by Surface Plasmon Resonance (SPR) or vice versa. The affinity of the binding is defined by the terms ka (rate constant for the association of the antibody from the antibody/antigen complex), kd (dissociation constant), and KD (kd/ka). Thus, binding means a specific and detectable interaction between the antibody and its cognate antigen, e.g., a binding affinity (KD) of 10E-4 M or less. “Specifically binding” means a binding affinity (KD) of 10E-8 M or less, in some embodiments of 10E-13 to 10E-8 M, in some embodiments of 10E-13 to 10E-9 M. “Effector functions” refer to those biological activities attributable to the Fc-region of an antibody, which vary with the antibody class. Examples of antibody effector functions include: C1q binding and complement dependent cytotoxicity (CDC); Fc receptor binding; antibody-dependent cell-mediated cytotoxicity (ADCC); phagocytosis; down regulation of cell surface receptors (e.g., B-cell receptor); and B-cell activation.

Fc receptor binding dependent effector functions can be mediated by the interaction of the Fc-region of an antibody with Fc receptors (FcRs), which are specialized cell surface receptors on hematopoietic cells. Fc receptors belong to the immunoglobulin superfamily and have been shown to mediate both the removal of antibody-coated pathogens by phagocytosis of immune complexes, and the lysis of erythrocytes and various other cellular targets (e.g., tumor cells) coated with the corresponding antibody, via antibody dependent cell mediated cytotoxicity (ADCC) (see e.g., Van de Winkel, J. G. and Anderson, C. L., J. Leukoc. Biol. 49 (1991) 511-524). FcRs are defined by their specificity for immunoglobulin isotypes: Fc receptors for IgG antibodies are referred to as FcγR. Fc receptor binding is described e.g., in Ravetch, J. V. and Kinet, J. P., Annu. Rev. Immunol. 9 (1991) 457-492; Capel, P. J., et al., Immunomethods 4 (1994) 25-34; de Haas, M., et al., J. Lab. Clin. Med. 126 (1995) 330-341; and Gessner, J. E., et al., Ann. Hematol. 76 (1998) 231-248.

Cross-linking of receptors for the Fc-region of IgG antibodies (FcγR) triggers a wide variety of effector functions including phagocytosis, antibody-dependent cellular cytotoxicity, and release of inflammatory mediators, as well as immune complex clearance and regulation of antibody production. In humans, three classes of FcγR have been characterized, which are:
FcγRI (CD64) binds monomeric IgG with high affinity and is expressed on macrophages, monocytes, neutrophils, and eosinophils. Modification in the Fc-region IgG at least at one of the amino acid residues E233-G236, P238, D265, N297, A327 and P329 (numbering according to EU index of Kabat) reduce binding to FcγRI. IgG2 residues at positions 233-236, substituted into IgG1 and IgG4, reduced binding to FcγRI by 10³-fold and eliminated the human monocyte response to antibody-sensitized red blood cells (Armour, K. L., et al., Eur. J. Immunol. 29 (1999) 2613-2624);
FcγRII (CD32) binds complexed IgG with medium to low affinity and is widely expressed. This receptor can be divided into two sub-types, FcγRIIA and FcγRIIB. FcγRIIA is found on many cells involved in killing (e.g., macrophages, monocytes, neutrophils) and seems able to activate the killing process. FcγRIIB seems to play a role in inhibitory processes and is found on B cells, macrophages and on mast cells and eosinophils. On B-cells, it seems to function to suppress further immunoglobulin production and isotype switching to, for example, the IgE class. On macrophages, FcγRIIB acts to inhibit phagocytosis as mediated through FcγRIIA. On eosinophils and mast cells, the B-form may help to suppress activation of these cells through IgE binding to its separate receptor. Reduced binding for FcγRIIA is found e.g., for antibodies comprising an IgG Fc-region with mutations at least at one of the amino acid residues E233-G236, P238, D265, N297, A327, P329, D270, Q295, A327, R292, and K414 (numbering according to EU index of Kabat);
FcγRIII (CD16) binds IgG with medium to low affinity and exists as two types. FcγRIIIA is found on NK cells, macrophages, eosinophils and some monocytes and T cells and mediates ADCC. FcγRIIIB is highly expressed on neutrophils. Reduced binding to FcγRIIIA is found e.g., for antibodies comprising an IgG Fc-region with mutation at least at one of the amino acid residues E233-G236, P238, D265, N297, A327, P329, D270, Q295, A327, S239, E269, E293, Y296, V303, A327, K338 and D376 (numbering according to EU index of Kabat).

Mapping of the binding sites on human IgG1 for Fc receptors, the above mentioned mutation sites and methods for measuring binding to FcγRI and FcγRIIA are described in Shields, R. L., et al., J. Biol. Chem. 276 (2001) 6591-6604.

An “effective amount” of an agent, e.g., a pharmaceutical formulation, refers to an amount effective, at dosages and for periods of time necessary, to achieve the desired therapeutic or prophylactic result.

The term “Fc receptor” as used herein refers to activation receptors characterized by the presence of a cytoplasmic ITAM sequence associated with the receptor (see e.g., Ravetch, J. V. and Bolland, S., Annu. Rev. Immunol. 19 (2001) 275-290). Such receptors are FcγRI, FcγRIIA and FcγRIIIA. The term “no binding of FcγR” denotes that at an antibody concentration of 10 μg/ml the binding of the antibody to NK cells is 10% or less of the binding found for anti-OX40L antibody LC.001 as reported in WO 2006/029879.

While IgG4 shows reduced FcR binding, antibodies of other IgG subclasses show strong binding. However Pro238, Asp265, Asp270, Asn297 (loss of Fc carbohydrate), Pro329 and 234, 235, 236 and 237 Ile253, Ser254, Lys288, Thr307, Gln311, Asn434, and His435 are residues which provide if altered also reduce FcR binding (Shields, R. L., et al. J. Biol. Chem. 276 (2001) 6591-6604; Lund, J., et al., FASEB J. 9 (1995) 115-119; Morgan, A., et al., Immunology 86 (1995) 319-324; and EP 0 307 434). In certain embodiments, the antibody according to the invention is of IgG1 or IgG2 subclass and comprises the mutation PVA236, GLPSS331, L234A/L235A or P329G/L234A/L235A. In certain embodiments, the antibody as reported herein is of IgG4 subclass and comprises the mutation L235E. In certain embodiments, the antibody according to the invention further comprises the mutation S228P.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3(L3)-FR4.

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

The term “hypervariable region” or “HVR”, as used herein, refers to each of the regions of an antibody variable domain comprising the amino acid residue stretches which are hypervariable in sequence (“complementarity determining regions” or “CDRs”) and/or form structurally defined loops (“hypervariable loops”), and/or contain the antigen-contacting residues (“antigen contacts”). Generally, antibodies comprise six HVRs; three in the VH (H1, H2, H3), and three in the VL (L1, L2, L3).

HVRs include
(a) hypervariable loops occurring at amino acid residues 26-32 (L1), 50-52 (L2), 91-96 (L3), 26-32 (H1), 53-55 (H2), and 96-101 (H3) (Chothia, C. and Lesk, A. M., J. Mol. Biol. 196 (1987) 901-917);
(b) CDRs occurring at amino acid residues 24-34 (L1), 50-56 (L2), 89-97 (L3), 31-35b (H1), 50-65 (H2), and 95-102 (H3) (Kabat, E. A. et al., Sequences of Proteins of Immunological Interest, 5th ed. Public Health Service, National Institutes of Health, Bethesda, MD (1991), NIH Publication 91-3242.);
(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745 (1996)); and
(d) combinations of (a), (b), and/or (c), including amino acid residues 46-56 (L2), 47-56 (L2), 48-56 (L2), 49-56 (L2), 26-35 (H1), 26-35b (H1), 49-65 (H2), 93-102 (H3), and 94-102 (H3).

Unless otherwise indicated, HVR residues and other residues in the variable domain (e.g., FR residues) are numbered herein according to Kabat et al., supra.

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., the CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

An “isolated” antibody is one, which has been separated from a component of its natural environment. In certain embodiments, an antibody is purified to greater than 95% or 99% purity as determined by, for example, electrophoretic (e.g., SDS-PAGE, isoelectric focusing (IEF), capillary electrophoresis) or chromatographic (e.g., size exclusion chromatography or ion exchange or reverse phase HPLC) analytical methods. For review of methods for assessment of antibody purity, see, e.g., Flatman, S. et al., J. Chrom. B 848 (2007) 79-87.

An “isolated” nucleic acid refers to a nucleic acid molecule that has been separated from a component of its natural environment.

An “isolated nucleic acid encoding an anti-human Abeta protein antibody” denotes to one or more nucleic acid molecules encoding the antibody heavy and light chains (or fragments thereof), including such nucleic acid molecule(s) in a single plasmid or separate plasmids.

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies and is not to be construed as requiring production of the antibody by any particular method. For example, the monoclonal antibodies in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

The term “package insert” is used to refer to instructions customarily included in commercial packages of therapeutic products, that contain information about the indications, usage, dosage, administration, combination therapy, contraindications and/or warnings concerning the use of such therapeutic products.

The term “pharmaceutical formulation” refers to a preparation which is in such form as to permit the biological activity of an active ingredient contained therein, e.g., of a therapeutic antibody, to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

As used herein, the term “treatment” (and grammatical variations thereof such as “treat” or “treating”) refers to clinical intervention in an attempt to alter the natural course of the individual being treated and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. For example, but not by way of limitation, such treatment can comprise an intervention to reduce the clinical decline in a subject with a disease associated with amyloid accumulation, e.g., Alzheimer's disease. In certain embodiments, such treatment can comprise converting an amyloid-positive subject to an amyloid-negative subject. In certain embodiments, such treatment can comprise an intervention to reduce the severity of at least one symptom associated with a disease associated with amyloid accumulation, e.g., Alzheimer's disease, in a subject. In certain embodiments, an antibody according to the current invention is used to delay or prevent development of a disease or to slow the progression of a disease.

The term “valent” as used within the current application denotes the presence of a specified number of binding sites in a (antibody) molecule. As such, the terms “bivalent”, “tetravalent”, and “hexavalent” denote the presence of two binding sites, four binding sites, and six binding sites, respectively, in a (antibody) molecule.

The term “variable region” or “variable domain” refers to the domain of an antibody heavy or light chain that is involved in binding of the antibody to its antigen. The variable domains of the heavy chain and light chain (VH and VL, respectively) of an antibody generally have similar structures, with each domain comprising four framework regions (FRs) and three hypervariable regions (HVRs) (see, e.g., Kindt, T. J. et al. Kuby Immunology, 6th ed., W.H. Freeman and Co., N.Y. (2007), page 91). A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano, S. et al., J. Immunol. 150 (1993) 880-887; Clackson, T. et al., Nature 352 (1991) 624-628). A (cognate) pair of an antibody heavy chain variable domain and an antibody light

When a range of values is listed herein, it is intended to encompass the boundaries as well as each value and sub-range within that range. For example, “2 mg/kg to 6 mg/kg” is intended to encompass, for example, 2.0 mg/kg, 2.5 mg/kg, 3 mg/kg, 3.5 mg/kg, 4 mg/kg, 4.5 mg/kg, 5 mg/kg, 5.5 mg/kg, 6 mg/kg, 2.5 mg/kg to 3 mg/kg, 2.5 mg/kg to 4.5 mg/kg, 3 mg/kg to 4.5 mg/kg, 4.5 mg/kg to 6 mg/kg, 2.5 mg/kg to 4 mg/kg, and so forth.

“Early AD” or “early Alzheimer's disease,” as used herein, is a continuum of AD severity from mild cognitive impairment due to AD-intermediate likelihood to mild Alzheimer's disease dementia. Subjects with early AD include subjects with mild Alzheimer's disease dementia. In certain embodiments, subjects with early AD have a score of 22 to 30 the minimal mental state examination (MMSA) and CDR (clinical dementia rating) global range of 0.5 to 1.0.

Subjects with “mild Alzheimer's disease dementia”, as used herein, are subjects that meet the National Institute on Aging and Alzheimer's Association (NIA-AA) core clinical criteria for probable Alzheimer's disease dementia in McKhann, G. M. et al., “The diagnosis of dementia due to Alzheimer's disease: Recommendations from the National Institute on Aging—Alzheimer's Association workgroups on diagnostic guidelines for Alzheimer's disease.” Alzheimer Dement. 7 (2011) 263-269. Also included herein are subjects who have a CDR score of 0.5 to 1.0 and a Memory Box score of 0.5 or greater at screening and baseline.

As used herein, “MMSE” refers to the Mini-Mental State Examination, a cognitive instrument commonly used for screening purposes, but also often measured longitudinally in AD clinical trials having a 30-point scale with higher scores indicating less impairment and lower scores indicating more impairment. As used herein, seven items measuring orientation to time and place, registration, recall, attention, language, and drawing were assessed. (Folstein, M. F., et al., J. Psychiatr. Res. 12 (1975) 189-198).

As used herein, “CDR-SB” refers to clinical dementia rating-sum of boxes. The CDR is a clinical scale that describes 5 degrees of impairment in performance on each of 6 categories of function including memory, orientation, judgment, and problem solving, community affairs, home and hobbies, and personal care. (Berg, L. et al., Ann. Neurol. 23 (1988) 477-484.) The ratings of degree of impairment obtained on each of the 6 categories of function are synthesized into 1 global rating of dementia CDR score (ranging from 0 to 3). A sum of boxes score provides an additional measure of change where each category has a maximum possible score of 3 points and the total score is a sum of the category scores giving a total possible score of 0 to 18 with higher scores indicating more impairment. The global score may be used as a clinical measure of severity of dementia.

As used herein, whether an early AD subject is “amyloid-positive” or “amyloid-negative” is determined based on whether or not the patient has a positive amyloid load as indicated by longitudinal PET assessment of an amyloid imaging agent uptake into the brain. In certain embodiments, a qualitative visual read of PET scans will be used to determine amyloid positive and amyloid negative by categorizing subjects as having either “normal” or “abnormal” uptake on the basis of the PET image pattern. Readers will have been trained and certified to recognize brain PET images with abnormal or normal patterns of uptake, or the detection of amyloid is done through a semi-quantitative or quantitative approach.

As used herein, the term “prevent” refers to obtaining beneficial or desired results including, but not limited to, prophylactic benefit. For prophylactic benefit, the composition may be administered to a subject at risk of developing Alzheimer's disease, to a subject having one or more preclinical symptoms but not clinical symptoms of Alzheimer's disease, or to a subject reporting one or more of the physiological symptoms of Alzheimer's disease, even though a clinical diagnosis of having Alzheimer's has not been made. As used herein “prevention” may further include therapeutic benefit, by which is meant eradication or amelioration of the underlying condition being treated or of one or more of the physiological symptoms associated therewith.

As used herein, the terms “serious adverse event” or “SAE” means an event that (1) results in death; (2) is life-threatening; (3) requires inpatient hospitalization or prolongation of existing hospitalization; (4) results in persistent or significant disability/incapacity; and/or (5) is a congenital anomaly/birth defect, which is observed after administration of a composition described herein.

The severity of a serious adverse event may be assessed based on a uniform scale used in the art. For example, the seriousness of a subject's serious adverse event may be evaluated according to the National Cancer Institute's “Common Terminology Criteria for Adverse Events” or “CTCAE.” The descriptions for the various CTCAE adverse event grades are set forth below:
Grade 1: mild; asymptomatic or mild symptoms; clinical or diagnostic observations only; intervention not indicated.
Grade 2: moderate; minimal, local, or noninvasive intervention indicated; limiting age-appropriate instrumental activities of daily living.
Grade 3: severe or medically significant but not immediately life-threatening; hospitalization or prolongation of hospitalization indicated; disabling; limiting self-care activities of daily living.
Grade 4: life-threatening consequences; urgent intervention indicated.
Grade 5: death related to the adverse event.

As used herein, the term “Centiloid” means a unit of the Centiloid Scale as described in Klunk, W. E., et al., Alz. Dement. 11 (2015) 1-15. As there has been considerable variability in the exact numbers reported as quantitative outcome measures of tracer retention a standardization of quantitative amyloid imaging measures by scaling the outcome of each particular analysis method or tracer to a 0 to 100 scale, anchored by young controls (less or 45 years) and typical Alzheimer's disease patients.

Thus, the Centiloid scale is a standardized PET-based amyloid load measurement that homogenizes amyloid load measurements across tracers and sites (see, e.g., Villain, N., et al., Rev. Neuro. 178 (2022) 1011-1030; Klunk, W. E., et al., Alz. Dement. 11 (2015) 1-15; Navitsky, M., et al. Alz. Dement. 14 (2018) 1565-1571).

Zero is the average value in “high certainty” amyloid-negative subjects (i.e., young subjects), and 100 is the average value in “typical” AD patients with dementia (Klunk, W. E., et al., Alz. Dement. 11 (2015) 1-15). The usual cut-off to define “amyloid-positive” and “amyloid-negative” individuals is around 10-25 Centiloids (de Souza, G. S., et al., Mol. Imag. Biol. 24 (2021) 394-403). This clearance, as measured with amyloid PET, is not an artifact related to the antibodies themselves, since a case report of autopsy data from a patient under aducanumab confirms this clearance (Plowey, E. D., et al., Acta Neuropathol. 144 (2022) 143-153).

For example, if the amyloid plaque level assessed by PET decreased below 25 Centiloids this indicates the removal of amyloid plaques under the pathological cut-off value.

It has been considered by some authors that the amyloid load needs to be lowered to 20 Centiloids or less to produce a noticeable cognitive benefit, with a lag time of several months between amyloid removal and clinical effect (Karran, E. and De Strooper, B., Nat. Rev. Drug. Discov. 21 (2022) 306-318).

As used herein, the term “PET” means Amyloid Positron Emission Tomography. Briefly, for determining amyloid load a standardized Centiloid approach with “ADNI-like” cortical regions and a whole cerebellum reference region was employed. This resulted in a single global measure of amyloid burden for each participant at each period.

The determined global reductions are deemed to be an appropriate measure for comparisons to other molecules that were or are in the clinic.

In more detail, participants were scanned with either florbetapir (trade name Amyvid) or florbetaben (trade name Neuraceq) with imaging protocol consistent with the FDA and EU label. Amyloid load was quantified via the Centiloid method (see, e.g., Klunk, W. E., et al., Alz. Dement. 11 (2015) 1-15).

For inclusion in the study, participants were required to have a minimum of 50 Centiloids (CL) to confirm amyloid pathology and sufficiently high burden to show possible treatment effect by trontinemab. (Note 100 CL represents the mean for a typical AD group, 0 CL is the mean for healthy controls with no amyloid, and 24 CL is considered the threshold of detectable amyloid differentiating no/sparse from moderate/frequent plaques (see, e.g., ref Navitsky, M., et al., Alzheimer's Dement. 14 (2018) 1565-1571). Navitsky et al. provided the conversion of florbetapir SUVr values to the Centiloid scale following the guidelines in Klunk et al.

Florbetapir was developed under the brand name Amyvid. It was approved by the FDA in 2012. It has a longer half-life and similar selectivity as the PIB compound (N-Methyl-[11C]2-(4′-Methylaminophenyl)-6-Hydroxybenzothiazole) and is thought to have a binding pattern similar to PIB with a high affinity for Abeta plaques (Anand, K. and Sabbagh, M., Neurotherap. 14 (2017) 54-61).

In more detail, Amyvid is a radioactive diagnostic agent for Positron Emission Tomography (PET) imaging of the brain to estimate β-amyloid neuritic plaque density in adult patients with cognitive impairment who are being evaluated for Alzheimer's Disease (AD) and other causes of cognitive decline. A negative Amyvid scan indicates sparse to no neuritic plaques, and is inconsistent with a neuropathological diagnosis of AD at the time of image acquisition; a negative scan result reduces the likelihood that a patient's cognitive impairment is due to AD.

A positive Amyvid scan indicates moderate to frequent amyloid neuritic plaques; neuropathological examination has shown this amount of amyloid neuritic plaque is present in patients with AD but may also be present in patients with other types of neurologic conditions as well as older people with normal cognition.

Generally, the dosage and administration is as follows:
The recommended dose for Amyvid is 370 MBq (10 mCi), maximum 50 pg mass dose, administered as a single intravenous bolus in a total volume of 10 mL or less. Follow the injection with an intravenous flush of 0.9% sterile sodium chloride.
Obtain 10-minute PET images starting approximately 30 to 50 minutes after intravenous injection (the patient should be supine, and the head positioned to center the brain, including the cerebellum, in the PET scanner field of view).
Image interpretation:
   The objective of Amyvid image interpretation is to provide an estimate of the brain β-amyloid neuritic plaque density.
   Images should be displayed in the transaxial orientation with access as needed to the sagittal and coronal planes. In reviewing the images, include all transaxial slices of the brain using a black-white scale with the maximum intensity of the scale set to the maximum intensity of all the brain pixels. Initially locate the brain slice with the highest levels of image contrast (highest radioactivity signals for Amyvid uptake) and adjust the contrast appropriately. Start image interpretation by displaying slices sequentially from the bottom of the brain to the top. Periodically refer to the sagittal and coronal plane image display, as needed to better define the radioactivity uptake and to ensure that the entire brain is displayed.
   Image interpretation is based upon the distribution of radioactive signal within the brain. Images are designated as positive or negative by comparing the radioactivity in cortical gray matter with activity in the adjacent white matter. This determination is made only in the cerebral cortex; the signal uptake in the cerebellum does not contribute to the scan interpretation (for example, a positive scan may show retained cerebellar gray-white contrast even when the cortical gray-white contrast is lost).
   Negative scans show more radioactivity in white matter than in gray matter, creating clear gray-white contrast.
   Positive scans show cortical areas with reduction or loss of the normally distinct gray-white contrast. These scans have one or more areas with increased cortical gray matter signal which results in reduced (or absent) gray-white contrast. Specifically, a positive scan will have either:
      a) Two or more brain areas (each larger than a single cortical gyrus) in which there is reduced or absent gray-white contrast. This is the most common appearance of a positive scan; or
      b) One or more areas in which gray matter radioactivity is intense and clearly exceeds radioactivity in adjacent white matter.

Piramal Imaging developed florbetaben under the brand name Neuraceq. This compound is an 18F-labeled polyethylene glycol stilbene derivative, which demonstrated high specificity for Abeta in vitro without binding to tau, frontotemporal lobe dementia tissue, or DLB tissue (Anand, K. and Sabbagh, M., Neurotherap. 14 (2017) 54-61).

Richards, D. and Sabbagh, M. N., reported about florbetaben for PET imaging of beta-amyloid plaques in the brain (Neurol. Ther. 3 (2014) 79-88). In more detail, they reported that PET scan reads on a subject-level (whole brain) were investigated to determine the sensitivity and specificity of florbetaben in this setting. The results from the visual assessment using the method applicable for clinical routine were compared with the neuropathological assessment of absence/presence of beta-amyloid plaques according to Consortium to Establish a Registry for Alzheimer's Disease (CERAD) criteria. A sensitivity of 100% (95% CI 80.5-100.0%), a specificity of 91.67% (95% CI 80.6-100.0%), and an almost perfect across-reader agreement [estimate interrater reliability (j)=0.870] was obtained for the first 31 deceased subjects and the image analyses from 10 young healthy volunteers who were considered to be negative for beta-amyloid, which results were subsequently confirmed in a larger Cohort of subjects and were used for approvals in the EU and US. Moreover, the addition of young, healthy volunteers was no longer needed as a sufficiently high number of amyloid-negative subjects became available for the analyses.

In more detail, Neuraceq™ is a radioactive diagnostic agent indicated for Positron Emission Tomography (PET) imaging of the brain to estimate B-amyloid neuritic plaque density in adult patients with cognitive impairment who are being evaluated for Alzheimer's Disease (AD) and other causes of cognitive decline. A negative Neuraceq scan indicates sparse to no neuritic plaques and is inconsistent with a neuropathological diagnosis of AD at the time of image acquisition; a negative scan result reduces the likelihood that a patient's cognitive impairment is due to AD. A positive Neuraceq scan indicates moderate to frequent amyloid neuritic plaques; neuropathological examination has shown this amount of amyloid neuritic plaque is present in patients with AD, but may also be present in patients with other types of neurologic conditions as well as older people with normal cognition

Generally, the dosage and administration is as follows:
The recommended dose of Neuraceq is 300 MBq (8.1 mCi), maximum 30 mcg mass dose, administered as a single slow intravenous bolus (6 sec/mL) in a total volume of up to 10 mL.
Acquire PET images over 15 to 20 minutes starting 45 to 130 minutes after Neuraceq injection (the patient should be kept supine with the head positioned to center the brain, including the cerebellum, in the PET scanner field of view).
Image Interpretation:
   The objective of Neuraceq image interpretation is to estimate β-amyloid neuritic plaque density in brain gray matter.
   PET images should be displayed in the transaxial orientation using gray scale or inverse gray scale. The sagittal and coronal planes may be used for additional orientation purposes. CT or MR images may be helpful for anatomic reference purposes. However, visual assessment should be performed using the axial planes according to the recommended reading methodology.
   Interpretation of the images is made by visually comparing the activity in cortical gray matter with activity in adjacent white matter. Regions displayed in the PET images which ‘anatomically’ correspond to white matter structures (e.g., the cerebellar white matter or the splenium) should be identified to help the readers orient themselves. Images should be viewed and assessed in a systematic manner, starting with the cerebellum and scrolling up through the lateral temporal and frontal lobes, the posterior cingulate cortex/precuneus, and the parietal lobes. For a gray matter cortical region to be assessed as showing ‘tracer uptake’, the majority of slices from the respective region must be affected.
   For each patient, the PET image assessment is categorized as either “β-amyloid-positive” or “β-amyloid-negative”. This determination is based on the assessment of tracer uptake in the gray matter of the following four brain regions: the temporal lobes, the frontal lobes, the posterior cingulate cortex/precuneus, and the parietal lobes; according to the following ‘rules for assessment:
      β-amyloid negative—tracer uptake (i.e., signal intensity) in gray matter is lower than in white matter in all four brain regions (no β-amyloid deposition)
      β-amyloid positive—smaller area(s) of tracer uptake equal to or higher than that present in white matter extending beyond the white matter rim to the outer cortical margin involving the majority of the slices within at least one of the four brain regions (“moderate” β-amyloid deposition), or a large confluent area of tracer uptake equal to or higher than that present in white matter extending beyond the white matter rim to the outer cortical margin and involving the entire region including the majority of slices within at least one of the four brain regions (“pronounced” β-amyloid deposition). There is no known clinical or histopathologic correlation distinguishing “moderate” from “pronounced” β-amyloid deposition.

As used herein, the term “ARIA” means Amyloid Related Imaging Abnormalities. Amyloid related imaging abnormalities (ARIA) are magnetic resonance imaging (MRI) abnormalities that represent the leakage of fluid or blood into the leptomeninges and/or parenchyma in the brain. ARIA-E (E for oedema or effusion) represents vasogenic oedema and/or sulcal effusion best seen on axial T2 FLAIR sequences and ARIA-H (H for hemosiderin [iron] deposits) represents microhaemorrhages or superficial siderosis best seen on iron sensitive sequences such as gradient echo T2* or susceptibility weighted imaging sequences (Sperling, R. A., et al., Alzheimers Dement. 7 (2011) 367-385; Hampel, H., et al., Brain 146 (2023) 4414-4424).

ARIA has been reported with antibodies capable of lowering Abeta aggregates, which led to the understanding that ARIA is a class effect of these agents (Salloway, S., et al., Neurol. 73 (2009) 2061-2070; Ostrowitzki, S., et al., Alz. Res. Ther. 9 (2017) 95). Although ARIA occurs mostly in association with anti-amyloid immunotherapy, it may occur spontaneously in people with Alzheimer's disease or in relation to other cerebrovascular and neurological conditions. The majority of patients who experience ARIA are asymptomatic. The most common symptoms can include headache, confusion, dizziness, nausea, and visual disturbances. These symptoms typically resolve following suspension of the anti-amyloid (Sperling, R., et al., Lancet Neurol. 11 (2012) 241-249; Sevigny, J., et al., Nature 537 (2016) 50-56; Ostrowitzki et al. 2017; Greenberg, S. M., et al. Nat. Rev. Neurol. 16 (2020) 30-42; Salloway, S., et al., JAMA Neurol. 79 (2022) 13-21).

The pathophysiological mechanism of ARIA is not fully understood but is thought to reflect temporarily increased vascular permeability due to increased trafficking of parenchymal amyloid beta (Abeta) to the perivascular space and/or leakage of blood vessels following the clearance of vascular Abeta. In the normal vasculature, smooth muscle cells are regularly distributed, but in patients with Alzheimer's Disease smooth muscle cell distribution is disrupted due to accumulation of Abeta pathology. Following anti-amyloid therapy, vessels may be susceptible to ARIA resulting in proteinaceous fluid and/or red blood cells leaking into the vessel wall and surrounding tissues. With Abeta clearance and recovery of the vascular structural integrity, the risk of ARIA could decrease (Sperling et al. 2012, supra; Barakos, J., et al., J. Prev. Alz. Dis. 9 (2022) 211-220).

### Trontinemab/RG6102

Trontinemab is a bispecific antibody comprising:
a) one full length antibody comprising two pairs each of a full length antibody light chain and a full length antibody heavy chain, wherein the binding sites formed by each of the pairs of the full length heavy chain and the full length light chain specifically bind to a first antigen, and
b) one additional Fab fragment, wherein the additional Fab fragment is fused to the C-terminus of one heavy chain of the full length antibody, wherein the binding site of the additional Fab fragment specifically binds to a second antigen,
wherein each of the full length antibody light chains comprises in the constant light chain domain (CL) at position 123 the amino acid residue arginine (instead of the wild-type glutamic acid residue; E123R mutation) and at position 124 the amino acid residue lysine (instead of the wild-type glutamine residue; Q124K mutation) (numbering according to Kabat),
wherein each of the full length antibody heavy chains comprises in the first constant heavy chain domain (CH1) at position 147 an glutamic acid residue (instead of the wild-type lysine residue; K147E mutation) and at position 213 an glutamic acid residue (instead of the wild-type lysine amino acid residue; K213E mutation) (numbering according to Kabat EU index),
wherein the additional Fab fragment specifically binding to the second antigen comprises a domain crossover such that the constant light chain domain (CL) and the constant heavy chain domain 1 (CH1) are replaced by each other, and
wherein the first antigen is human Abeta protein and the second antigen is human transferrin receptor.

In more detail, in one preferred embodiment, trontinemab is a bispecific antibody comprising a (full length) light chain that has the amino acid sequence of SEQ ID NO: 17, a (full length) heavy chain that has the amino acid sequence of SEQ ID NO: 18, a (full length) light chain that has the amino acid sequence of SEQ ID NO: 19, and an antibody Fab fragment comprising the amino acid sequences of SEQ ID NO: 20 (SEQ ID NO: 21 comprising SEQ ID NO: 18+linker+SEQ ID NO: 20).

SEQ ID NO: 17 has the amino acid sequence
DIVLTQSPATLSLSPGERATLSCRASQSVSSSYLAWYQQKPGQAPRLLIY
GASSRATGVPARFSGSGSGTDFTLTISSLEPEDFATYYCLQIYNMPITFG
QGTKVEIKRTVAAPSVFIFPPSDRKLKSGTASVVCLLNNFYPREAKVQWK
VDNALQSGNSQESVTEQDSKDSTYSLSSTLTLSKADYEKHKVYACEVTHQ
GLSSPVTKSENRGEC,
SEQ ID NO: 18 has the amino acid sequence
QVELVESGGGLVQPGGSLRLSCAASGFTFSSYAMSWVRQAPGKGLEWVSA
INASGTRTYYADSVKGRFTISRDNSKNTLYLQMNSLRAEDTAVYYCARGK
GNTHKPYGYVRYFDVWGQGTLVTVSSASTKGPSVFPLAPSSKSTSGGTAA
LGCLVEDYFPEPVTVSWNSGALTSGVHTFPAVLQSSGLYSLSSVVTVPSS
SLGTQTYICNVNHKPSNTKVDEKVEPKSCDKTHTCPPCPAPELLGGPSVF
LFPPKPKDTLMISRTPEVTCVVVDVSHEDPEVKENWYVDGVEVHNAKTKP
REEQYNSTYRVVSVLTVLHQDWLNGKEYKCKVSNKALPAPIEKTISKAKG
QPREPQVCTLPPSRDELTKNQVSLSCAVKGFYPSDIAVEWESNGQPENNY
KTTPPVLDSDGSFFLVSKLTVDKSRWQQGNVFSCSVMHEALHNHYTQKSL
SLSPG,
SEQ ID NO: 19 has the amino acid sequence
AIQLTQSPSSLSASVGDRVTITCRASQSISSYLAWYQQKPGKAPKLLIYR
ASTLASGVPSRFSGSGSGTDFTLTISSLQPEDFATYYCQQNYASSNVDNT
FGGGTKVEIKSSASTKGPSVFPLAPSSKSTSGGTAALGCLVKDYFPEPVT
VSWNSGALTSGVHTFPAVLQSSGLYSLSSVVTVPSSSLGTQTYICNVNHK
PSNTKVDKKVEPKSC
SEQ ID NO: 20 has the amino acid sequence
QSMQESGPGLVKPSQTLSLTCTVSGFSLSSYAMSWIRQHPGKGLEWIGYI
WSGGSTDYASWAKSRVTISKTSTTVSLKLSSVTAADTAVYYCARRYGTSY
PDYGDASGEDPWGQGTLVTVSSASVAAPSVFIFPPSDEQLKSGTASVVCL
LNNFYPREAKVQWKVDNALQSGNSQESVTEQDSKDSTYSLSSTLTLSKAD
YEKHKVYACEVTHQGLSSPVTKSFNRGEC
SEQ ID NO: 21 has the amino acid sequence
QVELVESGGGLVQPGGSLRLSCAASGFTFSSYAMSWVRQAPGKGLEWVSA
INASGTRTYYADSVKGRFTISRDNSKNTLYLQMNSLRAEDTAVYYCARGK
GNTHKPYGYVRYFDVWGQGTLVTVSSASTKGPSVFPLAPSSKSTSGGTAA
LGCLVEDYFPEPVTVSWNSGALTSGVHTFPAVLQSSGLYSLSSVVTVPSS
SLGTQTYICNVNHKPSNTKVDEKVEPKSCDKTHTCPPCPAPELLGGPSVF
LFPPKPKDTLMISRTPEVTCVVVDVSHEDPEVKENWYVDGVEVHNAKTKP
REEQYNSTYRVVSVLTVLHQDWLNGKEYKCKVSNKALPAPIEKTISKAKG
QPREPQVYTLPPCRDELTKNQVSLWCLVKGFYPSDIAVEWESNGQPENNY
KTTPPVLDSDGSFFLYSKLTVDKSRWQQGNVFSCSVMHEALHNHYTQKSL
SLSPGGGSGGGGSGGGGSGGGGSQSMQESGPGLVKPSQTLSLTCTVSGFS
LSSYAMSWIRQHPGKGLEWIGYIWSGGSTDYASWAKSRVTISKTSTTVSL
KLSSVTAADTAVYYCARRYGTSYPDYGDASGFDPWGQGTLVTVSSASVAA
PSVFIFPPSDEQLKSGTASVVCLLNNFYPREAKVQWKVDNALQSGNSQES
VTEQDSKDSTYSLSSTLTLSKADYEKHKVYACEVTHQGLSSPVTKSFNRG
EC
Abeta VH (SEQ ID NO: 04), CDRs underlined:
QVELVESGGG LVQPGGSLRL SCAASGFTFS SYAMSWVRQA
PGKGLEWVSA INASGTRTYY ADSVKGRFTI SRDNSKNTLY
LQMNSLRAED TAVYYCARGK GNTHKPYGYV RYFDVWGQGT
LVTVSS
CDR-H1 (SEQ ID NO: 01): GFTFSSYAMS
CDR-H2 (SEQ ID NO: 02): AINASGTRTY YADSVKG
CDR-H3 (SEQ ID NO: 03): GKGNTHKPYG YVRYFDV
Abeta VL (SEQ ID NO: 08), CDRs underlined:
DIVLTQSPAT LSLSPGERAT LSCRASQSVS SSYLAWYQQK
PGQAPRLLIY GASSRATGVP ARFSGSGSGT DFTLTISSLE
PEDFATYYCL QIYNMPITFG QGTKVEIK
CDR-L1 (SEQ ID NO: 05): RASQSVSSSY LA
CDR-L2 (SEQ ID NO: 06): GASSRAT
CDR-L3 (SEQ ID NO: 07): LQIYNMPIT
TfR VH (SEQ ID NO: 12), CDRs underlined:
QSMQESGPGL VKPSQTLSLT CTVSGFSLSS YAMSWIRQHP
GKGLEWIGYI WSGGSTDYAS WAKSRVTISK TSTTVSLKLS
SVTAADTAVY YCARRYGTSY PDYGDASGED PWGQGTLVTV SS
CDR-H1 (SEQ ID NO: 09): SYAMS
CDR-H2 (SEQ ID NO: 10): YIWSGGSTDY ASWAKS
CDR-H3 (SEQ ID NO: 11): RYGTSYPDYG DASGFDP
TfR VL (SEQ ID NO: 16), CDRs underlined:
AIQLTQSPSS LSASVGDRVT ITCRASQSIS SYLAWYQQKP
GKAPKLLIYR ASTLASGVPS RFSGSGSGTD FTLTISSLQP
EDFATYYCQQ NYASSNVDNT FGGGTKVEIK
CDR-L1 (SEQ ID NO: 13: RASQSISSYL A
CDR-L2 (SEQ ID NO: 14): RASTLAS
CDR-L3 (SEQ ID NO: 15): QQNYASSNVD NT

### Trontinemab is Characterized as Follows:

immunoglobulin [G1-VH-Ckappa_Vkappa-CH1]_[G1_kappa] with domain crossover, anti-[Homo sapiens APP (amyloid beta, A4 precursor protein) Abeta, conformational epitope 1-40/42)] and anti-[Homo sapiens TFRC (transferrin receptor, p90, CD71)], humanized monoclonal antibody, bispecific; fused chain G1-VH-Ckappa bispecific, anti-APP A beta and anti-TFRC (1-702) [gamma1 heavy chain anti-APP Abeta (1-455) humanized [VH (Homo sapiens IGHV3-23*04 (92.9%)-(IGHD)-IGHJ4*01 (93.3%), CDR-IMGT [8.8.19] (26-33.51-58.97-115)) (1-126)-Homo sapiens IGHG1*01, G1m17,1, G1v32 CH3 W22 (CHI K26>E (156), K119>E (222), K120 (223) (127-224), hinge 1-15 (225-239), CH2 (240-349), CH3 S10>C (363), D12 (365), L14 (367), T22>W (375) (350-454), CHS K2>del (455)) (127-455)]-18-mer diglycyl-seryl-tris(tetraglycyl-seryl) linker (456-473)-VH-C kappa chain anti-TFRC (474-702) humanized [VH (Homo sapiens IGHV4-31*02 (76.0%)-(IGHD)-IGHJ5*02 (100%), CDR-IMGT [8.7.19] (498-505.523-529.566-584)) (474-595)-alanyl-seryl (596-597)-C-KAPPA (Homo sapiens IGKC*01 (100%), Km3 A45.1 (641), V101 (679) (598-702)], (229-215′)-disulfide with kappa light chain anti-APP Abeta (1′-215′) humanized [V-KAPPA (Homo sapiens IGKV3D-7*01 (89.6%)-IGKJ1*01 (100%), CDR-IMGT [7.3.9] (27-33.51-53.90-98)) (1′-108′)-Homo sapiens IGKC*01 (100%), Km3 A45.1 (154), V101 (192), E12>R (124), Q13>K (125) (109′-215′)], (702-215″″)-disulfide with Vkappa-CH1 chain anti-TFRC (1″″-215″″) humanized [V-KAPPA (Homo sapiens IGKV1-13*02 (94.4%)-IGKJ4*01 (100%), CDR-IMGT [6.3.12] (27-32.50-52.89-100)) (1″″-110″″)-diseryl (111″″-112″″)-CH1-hinge (Homo sapiens IGHG1*01 CH1-h, G1m17 (CH1 K120 (209) (113″″-210″″), hinge 1-5 (211″″-215″″)) (113″″-215″″)], gamma1 heavy chain anti-APP Abeta (1″-456″) humanized [VH (Homo sapiens IGHV3-23*04 (92.9%)-(IGHD)-IGHJ4*01 (93.3%), CDR-IMGT [8.8.19] (26-33.51-58.97-115)) (1″-126″)-Homo sapiens IGHG1*01, G1m17,1, G1v33 CH3 S22, A24, V86 (CH1 K26>E (156), K119>E (222), K120 (223) (127″-224″), hinge 1-15 (225″-239″), CH2 (240″-349″), CH3 Y5>C (358), D12 (365), L14 (367), T22>S (375), L24>A (377), Y86>V (416) (350″-454″), CHS (455″-456″)) (127″-456″)], (229″-215′″)-disulfide with kappa light chain anti-APP Abeta (1′″-215′″) humanized [V-KAPPA (Homo sapiens IGKV3D-7*01 (89.6%)-IGKJ1*01 (100%), CDR-IMGT [7.3.9] (27-33.51-53.90-98)) (1′″-108′″)-Homo sapiens IGKC*01 (100%), Km3 A45.1 (154), V101 (192) E12>R (124), Q13>K (125) (109′″-215′″)], dimer (235-235″:238-238″:363-358″)-trisdisulfide, produced in Chinese hamster ovary (CHO) cells, glycoform alfa

| Heavy chain anti-APP beta 1-40/42, anti-TFRC (H) | |
|---|---|
| QVELVESGGG LVQPGGSLRL SCAASGFTFS SYAMSWVRQA PGKGLEWVSA | 50 |
| INASGTRTYY ADSVKGRFTI SRDNSKNTLY LQMNSLRAED TAVYYCARGK | 100 |
| GNTHKPYGYV RYFDVWGQGT LVTVSSASTK GPSVFPLAPS SKSTSGGTAA | 150 |
| LGCLVEDYFP EPVTVSWNSG ALTSGVHTFP AVLQSSGLYS LSSVVTVPSS | 200 |
| SLGTQTYICN VNHKPSNTKV DEKVEPKSCD KTHTCPPCPA PELLGGPSVF | 250 |
| LFPPKPKDTL MISRTPEVTC VVVDVSHEDP EVKFNWYVDG VEVHNAKTKP | 300 |
| REEQYNSTYR VVSVLTVLHQ DWLNGKEYKC KVSNKALPAP IEKTISKAKG | 350 |
| QPREPQVYTL PPCRDELTKN QVSLWCLVKG FYPSDIAVEW ESNGQPENNY | 400 |
| KTTPPVLDSD GSFFLYSKLT VDKSRWQQGN VFSCSVMHEA LHNHYTQKSL | 450 |
| SLSPGGGSGG GGSGGGGSGG GGSQSMQESG PGLVKPSQTL SLTCTVSGFS | 500 |
| LSSYAMSWIR QHPGKGLEWI GYIWSGGSTD YASWAKSRVT ISKTSTTVSL | 550 |
| KLSSVTAADT AVYYCARRYG TSYPDYGDAS GFDPWGQGTL VTVSSASVAA | 600 |
| PSVFIFPPSD EQLKSGTASV VCLLNNFYPR EAKVQWKVDN ALQSGNSQES | 650 |
| VTEQDSKDST YSLSSTLTLS KADYEKHKVY ACEVTHQGLS SPVTKSFNRG | 700 |
| EC | 702 |
| Heavy chain anti-APP A beta 1-40/42 (H″) | |
| QVELVESGGG LVQPGGSLRL SCAASGFTFS SYAMSWVRQA PGKGLEWVSA | 50 |
| INASGTRTYY ADSVKGRFTI SRDNSKNTLY LQMNSLRAED TAVYYCARGK | 100 |
| GNTHKPYGYV RYFDVWGQGT LVTVSSASTK GPSVFPLAPS SKSTSGGTAA | 150 |
| LGCLVEDYFP EPVTVSWNSG ALTSGVHTFP AVLQSSGLYS LSSVVTVPSS | 200 |
| Light chain Vkappa-CH1, anti-TFRC (L″″) | |
| AIQLTQSPSS LSASVGDRVT ITCRASQSIS SYLAWYQQKP GKAPKLLIYR | 50 |
| ASTLASGVPS RFSGSGSGTD FTLTISSLOP EDFATYYCQQ NYASSNVDNT | 100 |
| FGGGTKVEIK SSASTKGPSV FPLAPSSKST SGGTAALGCL VKDYFPEPVT | 150 |
| VSWNSGALTS GVHTFPAVLQ SSGLYSLSSV VTVPSSSLGT QTYICNVNHK | 200 |
| PSNTKVDKKV EPKSC | 215 |

Disulfide bridges location

| | | | | | | |
|---|---|---|---|---|---|---|
| Intra-H (C23-C104) | 22-96 | 153-209 | 270-330 | 376-434 | 494-565 | 622-682 |
| | 22″-96″ | 153″-209″ | 270″-330″ | 376″-434″ | | |
| Intra-L (C23-C104) | 23′-89′ | 135′-195′ | | | | |
| | 23″′-89″′ | 135″′-195″′ | | | | |
| | 23″″-88″″ | 139″″-195″″ | | | | |

| | | | | |
|---|---|---|---|---|
| Inter-H-L (h 5-CL 126) | 229-215′ | 229″-215″′ | (CL126-h 5) | 702-215″″ |
| Inter-H-H (h 11, h 14) | 235-235″ | 238-238″ | (CH3 S10 > C—CH3 | 363-358″ |
| | | | Y5 > C) | |

N-terminal glutaminyl cyclization
H VH Q1>pyroglutamyl (pE, 5-oxoprolyl): 1, 1″
N-glycosylation sites
H VH N57: 52, 52″
Glycosylated
H CH2 N84.4: 306, 306″
Fucosylated complex bi-antennary CHO-type glycans
C-terminal lysine clipping
H CHS K2: 456″

Trontinemab targets fibrillar Abeta protein as well as amyloid plaques and also oligomers which means that trontinemab is in general suitable to do both: clearing plaques and preventing re-accumulation as well as neutralizing oligomers.

### Comparative Anti-Abeta Monoclonal Antibodies

Zhao et al. (Int. J. Nanomed. 18 (2023) 7825-7845) summarized the clinical advancement of anti-Abeta antibodies as follows:
aducanumab
   target residues 3 to 7 in the Abeta protein N-terminus;
   exhibits low monovalent affinity but strong avidity for epitope-rich aggregates, allowing it to selectively bind to Abeta protein oligomeric or fibrillar aggregates; this binding activates microglia;
   Phase III trials for efficacy: EMERGE (NCT02484547) and ENGAGE (NCT02477800); high-dose aducanumab in the EMERGE trial met its primary endpoint, though the low-dose aducanumab did not exhibit significant benefits compared to the placebo group; the ENGAGE trial did not meet its primary endpoint;
   about a third of aducanumab-treated participants developed amyloid-related imaging abnormalities (ARIA), of which a quarter were symptomatic and 3% were severe; ARIA-E (vasogenic edema) and ARIA-H (microhemorrhages and hemosiderosis) were commonly observed in patients receiving a dosage of 10 mg/kg, with a 19.1% and 35% incidence rate, respectively; APOE ε4 carriers had a higher incidence compared to non-carriers;
lecanemab
   selectively binding to soluble Abeta protein aggregates (including oligomers and protofibrils), approximately 1000 times greater than Abeta protein monomers and 10-15 times than insoluble fibrils;
   Phase III CLARITY AD trial (NCT03887455); intravenous lecanemab at a dosage of 10 mg/kg every two weeks; assessing clinical decline using CDR-SB as the primary endpoint, the lecanemab group showed a substantial reduction in clinical decline; ARIA-H and ARIA-E findings
   despite its moderate therapeutic effect, lecanemab failed to demonstrate clinically significant benefits in patients with clinically manifest or prodromal dementia;
   emerging evidence from the Phase 2 lecanemab study shows that fluid biomarkers re-emerge after stopping treatment with conventional amyloid depleting monoclonal antibodies; plasma Aβ42/40 ratio begins decreasing again, plasma p-tau181 starts increasing; these biomarkers precede the slow re-accumulation of amyloid PET/brain amyloid; the biomarker observations were associated with clinical decline as measured via CDR-SB, ADCOMS and ADAS-cog; the rates of clinical progression after stopping treatment were similar in lecanemab and placebo treated participants;
   lecanemab, marketed as Leqembi, prescribing information directs administering 10 mg/kg lecanemab once every two weeks as a one-hour intravenous infusion and includes a warning section that recommends enhanced clinical vigilance for amyloid-related imaging abnormalities (“ARIA”) during the first 14 weeks of treatment with Leqembi and describes an increased risk of ARIA in apolipoprotein E F4 (“ApoE4”) homozygotes compared to heterozygotes and non-carriers;
donanemab
   specifically targets a pyroglutamated form of Abeta protein (N3pG-Aβ) that is aggregated in amyloid plaques;
   Phase III trial, TRAILBLAZER-ALZ 2 (NCT04437511); 72-week period; donanemab treatment every four weeks; donanemab was reported to be associated with the occurrence of ARIA, with approximately 40% of participants in donanemab groups experiencing ARIA, and about 26.1% of them showing symptomatic ARIA (symptomatic ARIA-E was observed in only 0.8% of participants receiving placebo);
remternetug
   specifically targets N3pG-Aβ;
ABBV-916
   recognizes the N3pG-AP aggregated in amyloid plaques;
ACU193
   Abeta protein oligomers-selective antibody;
   Phase I clinical trial (NCT04931459) showed significant reductions in amyloid plaque levels as detectable by brain amyloid PET scans in patients receiving higher doses of ACU193 after 6-12 weeks; 10.4% of patients experiencing ARIA-E, of which 2.1% were symptomatic.

In clinical settings, the binding of anti-Aβ mAbs to amyloid plaques has been associated with a higher incidence of ARIA.

| | | | clinical | |
|---|---|---|---|---|
| antibody | target | indication | effect | ARIA |
| aducanumab | Oligomeric Aβ | Mild AD | high-dose | about a third of |
| | or fibrillar | | aducanumab | aducanumab-treated |
| | aggregates; | | in the | participants developed |
| | Aducanumab- | | EMERGE | amyloid-related imaging |
| | avwa is a | | trial met its | abnormalities (ARIA), of |
| | recombinant | | primary | which a quarter were |
| | human | | endpoint, | symptomatic and 3% were |
| | immunoglobulin | | though the | severe; |
| | gamma 1 (IgG1) | | low-dose | ARIA-E (vasogenic |
| | monoclonal | | aducanumab | edema) and ARIA-H |
| | antibody | | did not | (microhemorrhages and |
| | directed against | | exhibit | hemosiderosis) were |
| | aggregated | | significant | commonly observed in |
| | soluble and | | benefits | patients receiving a dosage |
| | insoluble forms | | compared to | of 10 mg/kg, with a 19.1% |
| | of amyloid beta, | | the placebo | and 35% incidence rate, |
| | and is | | group | respectively. Besides, |
| | expressed in a | | | APOE ε4 carriers had a |
| | Chinese hamster | | | higher incidence compared |
| | ovary cell line. | | | to noncarriers. |
| | Aducanumab- | | | |
| | avwa has an | | | |
| | approximate | | | |
| | molecular | | | |
| | weight of 146 | | | |
| | kDa; | | | |
| | Aducanumab- | | | |
| | avwa is a | | | |
| | human, | | | |
| | immunoglobulin | | | |
| | gamma 1 (IgG1) | | | |
| | monoclonal | | | |
| | antibody | | | |
| | directed | | | |
| | against | | | |
| | aggregated | | | |
| | soluble and | | | |
| | insoluble forms | | | |
| | of amyloid beta. | | | |
| lecanemab | Soluble Aβ | MCI or mild | Phase II: | ARIA-H and ARIA-E, |
| | protofibrils | AD | showed that | |
| | | | patients | |
| | | | receiving | |
| | | | lecanemab | |
| | | | at a dosage | |
| | | | of 10 mg/kg | |
| | | | twice a | |
| | | | month | |
| | | | experienced | |
| | | | a | |
| | | | statistically | |
| | | | significant | |
| | | | reduction in | |
| | | | cognitive | |
| | | | decline at 6, | |
| | | | 12, and 18 | |
| | | | months. | |
| | | | This dosage | |
| | | | led to a 93% | |
| | | | reduction in | |
| | | | brain | |
| | | | amyloid | |
| | | | plaques, | |
| | | | a 47% | |
| | | | slower | |
| | | | decline in | |
| | | | cognitive | |
| | | | function as | |
| | | | measured | |
| | | | by ADAS- | |
| | | | Cog, and a | |
| | | | 30% slower | |
| | | | decline in | |
| | | | cognitive | |
| | | | function | |
| | | | as measured | |
| | | | by | |
| | | | ADCOMS. | |
| | | | Phase III: | |
| | | | months, | |
| | | | with | |
| | | | reduced | |
| | | | amyloid | |
| | | | plaque | |
| | | | burden, a | |
| | | | 24% | |
| | | | deceleration | |
| | | | in disease | |
| | | | progression, | |
| | | | and a 37% | |
| | | | deceleration | |
| | | | in decline in | |
| | | | activities of | |
| | | | daily living | |
| donanemab | N3pG-Aβ Aβ | Early | treatment | approximately 40% of |
| | plaques | symptomatic | every four | participants in donanemab |
| | | AD | weeks. At | groups experiencing ARIA, |
| | | | the 76-week | and about 26.1% of them |
| | | | mark, | showing symptomatic |
| | | | donanemab | ARIA. In contrast, |
| | | | treatment | symptomatic ARIA-E was |
| | | | showed a | observed in only 0.8% of |
| | | | 35.1% | participants receiving |
| | | | reduction in | placebo. |
| | | | disease | |
| | | | progression | |
| | | | risk | |
| | | | compared to | |
| | | | placebo in | |
| | | | early AD | |
| | | | patients | |
| | | | with | |
| | | | low/medium | |
| | | | tau | |
| | | | pathology, | |
| | | | as measured | |
| | | | by iADRS | |
| | | | scores. | |
| remternetug | N3pG-Aβ Aβ | Early-stage | Phase III | |
| | plaques | AD | ongoing | |
| ABBV-916 | N3pG-Aβ Aβ | Early-stage | Phase II to | |
| | plaques | AD | be | |
| | | | completed | |
| | | | in | |
| | | | December | |
| | | | 2024 | |
| ACU193 | Aβ oligomers | MCI or | Phase I: | 10.4% of patients |
| | | Early-stage | reductions | experiencing ARIA-E, of |
| | | AD | in amyloid | which 2.1% were |
| | | | plaque | symptomatic. |
| | | | levels as | |
| | | | detectable | |
| | | | by brain | |
| | | | amyloid | |
| | | | PET scans | |
| | | | in patients | |
| | | | receiving | |
| | | | higher doses | |
| | | | of ACU193 | |
| | | | after 6-12 | |
| | | | weeks | |
| trontinemab | Aβ oligomers, | Mild to | | |
| | fibers, and | moderate | | |
| | plaques | AD | | |

| | | | |
|---|---|---|---|
| Aducanumab | Dose | 10 mg/kg Q4W IV | Titration to 10 mg/kg |
| | | | Q4W IV |
| | ARIA-E/PET | 41%/−19%* (Wk54) | 35%/−60.8 Centiloids |
| | | | (Wk78) |
| Gantenerumab | Dose | 225 mg Q4W SC | Titration to 510 mg Q2W |
| | | | SC |
| | ARIA-E/PET | 13.5%/−4.8%* | 25%/−57.4 CL (Wk116) |
| | | (Wk100) | |
| Lecanemab | Dose | 10 mg/kg Q2W IV | 10 mg/kg Q2W IV |
| | ARIA-E/PET | 9.9%/−22%* | 13%/−59.1 CL (Wk78) |
| | | (Wk78) | |
| Donanemab | Dose | Titration to 1400 | Titration to 1400 mg Q4W |
| | | mg Q4W IV | IV |
| | ARIA-E/PET | 26.7/−85 CL | 24%/−88 CL (Wk76) |
| | | (Wk76) | |

*Mean percent change from baseline in SUVR; Table shows highest dose tested in respective trial only

### Amyloid Reduction and Clinical Effect

Recent efforts have focused on targeting amyloid (Bachurin, S. O., et al., Med. Res. Rev. 37 (2017) 1186-1225) as the most compelling therapeutic target (Graham, W. V., et al., Annu. Rev. Med. 68 (2017) 413-430). These therapies are based on the amyloid hypothesis that postulates that Abeta accumulation is the primary factor driving Abeta pathogenesis (Selkoe, D. J., Neuron. 6 (1991) 487-498; Hardy, J. and Selkoe, D. J., Science 297 (2002) 353-356; Selkoe and Hardy 2016, supra). Several monoclonal anti-Abeta antibodies, including aducanumab, lecanemab, donanemab, and gantenerumab have been shown to bind Abeta protein, promote its clearance, reduce both the deposition of Abeta aggregates, as well as the markers of neurodegeneration in the CSF in the clinical setting (Salloway et al. 2009, supra; Ostrowitzki, S., et al., Arch. Neurol. 69 (2012) 198-207; Sevigny et al 2016, supra; Klein, G., et al., J. Prev. Alz. Dis. 8 (2021) 3-6; Klein, G., et al., Alz. Res. Ther. 11 (2019) 101; Bateman, R. J., et al., N. Engl. J. Med. 389 (2023) 1862-1876; Small, S. A. and Swanson, L. W., Cold Spring Harb. Symp. Quant. Biol. 83 (2018) 193-200; Budd Haeberlein, S., et al. J. Prev. Alz. Dis. 9 (2022) 197-210; Bateman AAT-AD/PD 2020; Ostrowitzki et al 2017, supra; Bateman AAT-AD/PD 2020).

In addition, a Phase II study with the amyloid plaque targeting therapy donanemab met its primary efficacy endpoint and demonstrated an extensive reduction of amyloid plaques along with regional lowering of the tau load as revealed by amyloid and tau PET imaging after 76 weeks of treatment.

The potential link between dose-dependent amyloid removal and clinical efficacy is further supported by the results of a large Phase II study with lecanemab as it demonstrated a substantial amyloid reduction for the highest dose and a dose-dependent clinical benefit on various scales of cognition (Swanson et al. 2018, supra). This was confirmed in a large Phase III study (CLARITY AD) with lecanemab, which met its primary endpoint. Lecanemab was found to reduce the CDR-SB score more efficiently compared with placebo at 18 months. The study also achieved all secondary endpoints, indicating the effectiveness of lecanemab in early AD (Van Dyck et al., NEJM 388 (2022) 9-21).

Thus, targeting amyloid deposits represents one of the most promising avenues for the treatment of AD. Preclinical, a large body of evidence suggests that targeting Abeta aggregates in transgenic models of AD with vaccination with Abeta or passive immunization with anti-Abeta antibodies resulted in decreased amyloidosis and in improvement of cognitive function (Bard, F., et al. Nat. Med. 6 (2000) 916-919; Janus, C., et al., Biochim. Biophys. Acta 1502 (2000) 63-75). The results obtained clinically with the 4 antibodies gantenerumab, lecanemab, aducanumab, and donanemab support a potential link between amyloid removal and clinical efficacy. In a Phase I study with aducanumab in patients with early AD, slowing of clinical decline was associated with a time and dose-related reduction of deposited amyloid, as seen on brain amyloid PET imaging (Sevigny et al. 2016, supra). Similarly, in a recent publication (McDade, E., et al., Alz. Res. Ther. 14 (2022) 191), a significant correlation between the level of amyloid removal achieved by different doses of lecanemab and progression on various clinical scales was shown.

Clinically, a significant effect on preventing clinical decline has been shown with anti-amyloid antibodies clearing amyloid plaques rapidly, and with a high proportion of participants reaching amyloid negativity at the end of the double blind treatment period. For example, lecanemab was able to bring 81% of the participants below the reference amyloid positivity threshold of 30 Centiloids threshold after 18 months of treatment. Overall, a mean amyloid PET level of 22.99 Centiloids was achieved in the lecanemab group after 18 months of treatment was achieved (van Dyck, C. H., et al., N. Eng. J. Med. 388 (2023) 9-21). Donanemab achieved similar results, with 72% of the participants being falling below the amyloid positivity threshold of 24.1 Centiloids after 18 months (Eli Lilly and Company 2023) and, similar to lecanemab, met its primary clinical endpoint in Phase II. GRADUATE I and GRADUATE II, the two pivotal Phase II studies with gantenerumab did not meet their primary endpoint. However, the magnitude of amyloid plaque removal was smaller than expected, predicted based on the open label extension (OLE) study data from Scarlet RoAD and Marguerite RoAD, two historical Phase III studies (ScarletRoAD and MargueriteRoAD) of gantenerumab, in which robust and sustained Abeta plaque reduction, comparable with the levels achieved by other amyloid lowering antibodies, had been demonstrated in the OLE.

Based on these results, bringing amyloid levels below the positivity threshold rapidly appears to be a necessary feature to achieve clinical efficacy (Karran, E. and De Strooper, B., Nat. Rev. Drug. Discov. 21 (2022) 306-318). The extent and speed of amyloid plaque removal by anti-amyloid antibodies are both dose and exposure dependent. Because of their large size, only about 0.1%-0.2% of peripherally administered antibodies cross the intact BBB (Poduslo, J. F., et al., Proc. Natl. Acad. Sci. USA 91 (1994) 5705-5709; Yu, Y. J. and Watts, R. J., Neurotherap. 10 (2013) 459-472).

Additionally, the rate of occurrence of any ARIA, ARIA-E, or ARIA-H was significantly increased by high-clearance anti-amyloid immunotherapies (Villain, N., et al., Rev. Neuro. 178 (2022) 1011-1030).

Shcherbinin et al. (JAMA Neurology 79 (2022) 1015-1024) reported that slowing of tau pathology (Tau PET) at 76 weeks was more pronounced with complete amyloid clearance at 24 weeks as found in the Phase II clinical trial of donanemab.

A meta-analysis of the aducanumab, donanemab and lecanemab clinical trial data by Villain, N., et al. (Rev. Neuro. 178 (2022) 1011-1030) supported the assertion that those clinical trial data were adequate on their own to convincingly demonstrate a clinical benefit in reducing the clinical decline in patients with Alzheimer disease and proved a significant global effect of high-clearance anti-amyloid immunotherapies on the progression of CDR-SB, ADAS-Cog, but not of MMSE. Thus, it confirms the trend regarding the clinical efficacy of high-clearance anti-amyloid immunotherapies and offers the opportunity to estimate the magnitude of the clinical effect of high-clearance anti-amyloid immunotherapies (Liu, K. Y. and Howard, R., Nat. Rev. Neurol. 17 (2021) 715-722; Liu, K. Y., et al., Lancet Psychiatry 8 (2021) 1013-1016).

### Therapeutically Effective Amount of the at Least One Bispecific Anti-Abeta/TfR Antibody

The methods of the present invention, in certain embodiments, comprise administering to a subject a composition comprising a therapeutically effective amount of at least one bispecific anti-Abeta/TfR antibody. As used herein, the term a “therapeutically effective amount” refers to an amount of a compound or pharmaceutical composition sufficient to produce a desired therapeutic effect.

One of ordinary skill in the art will understand that the therapeutically effective amount of the at least one bispecific anti-Abeta/TfR antibody administered to a subject may depend upon a number of factors including pharmacodynamic characteristics, route of administration, frequency of treatment, and health, age, and weight of the subject to be treated and, with the information disclosed herein, will be able to determine the appropriate amount for each subject.

In certain embodiments, the therapeutically effective amount is a dose chosen to improve efficacy and/or maintain efficacy and improve at least one of safety and tolerability. In some embodiments, the therapeutically effective amount is chosen to lower at least one side effect and simultaneously improve efficacy and/or maintain efficacy.

In certain embodiments, 1 mg/kg to 9 mg/kg, 1 mg/kg to 8 mg/kg, 1 mg/kg to 7.2 mg/kg, 1 mg/kg to 6.5 mg/kg, 1 mg/kg to 6 mg/kg, 1 mg/kg to 5.5 mg/kg, 1 mg/kg to 4 mg/kg, 1 mg/kg to 3.9 mg/kg, 1 mg/kg to 3.8 mg/kg, 1 mg/kg to 3.7 mg/kg, or 1 mg/kg to 3.6 mg/kg of at least one bispecific anti-Abeta/TfR antibody is administered to the subject relative to the body weight of the subject.

In certain embodiments, 1.2 mg/kg to 9 mg/kg, 1.2 mg/kg to 8 mg/kg, 1.2 mg/kg to 7.2 mg/kg, 1.2 mg/kg to 6.5 mg/kg, 1.2 mg/kg to 6 mg/kg, 1.2 mg/kg to 5.5 mg/kg, 1.2 mg/kg to 4 mg/kg, 1.2 mg/kg to 3.9 mg/kg, 1.2 mg/kg to 3.8 mg/kg, 1.2 mg/kg to 3.7 mg/kg, or 1.2 mg/kg to 3.6 mg/kg of at least one bispecific anti-Abeta/TfR antibody is administered to the subject relative to the body weight of the subject.

In certain embodiments, 1.5 mg/kg to 9 mg/kg, 1.5 mg/kg to 8 mg/kg, 1.5 mg/kg to 7.2 mg/kg, 1.5 mg/kg to 6.5 mg/kg, 1.5 mg/kg to 6 mg/kg, 1.5 mg/kg to 5.5 mg/kg, 1.5 mg/kg to 4 mg/kg, 1.5 mg/kg to 3.9 mg/kg, 1.5 mg/kg to 3.8 mg/kg, 1.5 mg/kg to 3.7 mg/kg, or 1.5 mg/kg to 3.6 mg/kg of at least one bispecific anti-Abeta/TfR antibody is administered to the subject relative to the body weight of the subject.

In certain embodiments, 1.8 mg/kg to 9 mg/kg, 1.8 mg/kg to 8 mg/kg, 1.8 mg/kg to 7.2 mg/kg, 1.8 mg/kg to 6.5 mg/kg, 1.8 mg/kg to 6 mg/kg, 1.8 mg/kg to 5.5 mg/kg, 1.8 mg/kg to 4 mg/kg, 1.8 mg/kg to 3.9 mg/kg, 1.8 mg/kg to 3.8 mg/kg, 1.8 mg/kg to 3.7 mg/kg, or 1.8 mg/kg to 3.6 mg/kg of at least one bispecific anti-Abeta/TfR antibody is administered to the subject relative to the body weight of the subject.

In certain embodiments, 2 mg/kg to 9 mg/kg, 2 mg/kg to 8 mg/kg, 2 mg/kg to 7.2 mg/kg, 2 mg/kg to 6.5 mg/kg, 2 mg/kg to 6 mg/kg, 2 mg/kg to 5.5 mg/kg, 2 mg/kg to 4 mg/kg, 2 mg/kg to 3.9 mg/kg, 2 mg/kg to 3.8 mg/kg, 2 mg/kg to 3.7 mg/kg, or 2 mg/kg to 3.6 mg/kg of at least one bispecific anti-Abeta/TfR antibody is administered to the subject relative to the body weight of the subject.

In certain embodiments, 2.2 mg/kg to 9 mg/kg, 2.2 mg/kg to 8 mg/kg, 2.2 mg/kg to 7.2 mg/kg, 2.2 mg/kg to 6.5 mg/kg, 2.2 mg/kg to 6 mg/kg, 2.2 mg/kg to 5.5 mg/kg, 2.2 mg/kg to 4 mg/kg, 2.2 mg/kg to 3.9 mg/kg, 2.2 mg/kg to 3.8 mg/kg, 2.2 mg/kg to 3.7 mg/kg, or 2.2 mg/kg to 3.6 mg/kg of at least one bispecific anti-Abeta/TfR antibody is administered to the subject relative to the body weight of the subject.

In certain embodiments, 2.4 mg/kg to 9 mg/kg, 2.4 mg/kg to 8 mg/kg, 2.4 mg/kg to 7.2 mg/kg, 2.4 mg/kg to 6.5 mg/kg, 2.4 mg/kg to 6 mg/kg, 2.4 mg/kg to 5.5 mg/kg, 2.4 mg/kg to 4 mg/kg, 2.4 mg/kg to 3.9 mg/kg, 2.4 mg/kg to 3.8 mg/kg, 2.4 mg/kg to 3.7 mg/kg, or 2.4 mg/kg to 3.6 mg/kg of at least one bispecific anti-Abeta/TfR antibody is administered to the subject relative to the body weight of the subject.

In certain embodiments, 2.6 mg/kg to 9 mg/kg, 2.6 mg/kg to 8 mg/kg, 2.6 mg/kg to 7.2 mg/kg, 2.6 mg/kg to 6.5 mg/kg, 2.6 mg/kg to 6 mg/kg, 2.6 mg/kg to 5.5 mg/kg, 2.6 mg/kg to 4 mg/kg, 2.6 mg/kg to 3.9 mg/kg, 2.6 mg/kg to 3.8 mg/kg, 2.6 mg/kg to 3.7 mg/kg, or 2.6 mg/kg to 3.6 mg/kg of at least one bispecific anti-Abeta/TfR antibody is administered to the subject relative to the body weight of the subject.

In certain embodiments, 2.8 mg/kg to 9 mg/kg, 2.8 mg/kg to 8 mg/kg, 2.8 mg/kg to 7.2 mg/kg, 2.8 mg/kg to 6.5 mg/kg, 2.8 mg/kg to 6 mg/kg, 2.8 mg/kg to 5.5 mg/kg, 2.8 mg/kg to 4 mg/kg, 2.8 mg/kg to 3.9 mg/kg, 2.8 mg/kg to 3.8 mg/kg, 2.8 mg/kg to 3.7 mg/kg, or 2.8 mg/kg to 3.6 mg/kg of at least one bispecific anti-Abeta/TfR antibody is administered to the subject relative to the body weight of the subject.

In certain embodiments, 3 mg/kg to 9 mg/kg, 3 mg/kg to 8 mg/kg, 3 mg/kg to 7.2 mg/kg, 3 mg/kg to 6.5 mg/kg, 3 mg/kg to 6 mg/kg, 3 mg/kg to 5.5 mg/kg, 3 mg/kg to 4 mg/kg, 3 mg/kg to 3.9 mg/kg, 3 mg/kg to 3.8 mg/kg, 3 mg/kg to 3.7 mg/kg, or 3 mg/kg to 3.6 mg/kg of at least one bispecific anti-Abeta/TfR antibody is administered to the subject relative to the body weight of the subject.

In certain embodiments, 1 mg/kg to 9 mg/kg, 1.2 mg/kg to 9 mg/kg, 1.4 mg/kg to 9 mg/kg, 1.8 mg/kg to 9 mg/kg, 1.9 mg/kg to 9 mg/kg, 2 mg/kg to 9 mg/kg, 2.1 mg/kg to 9 mg/kg, 2.2 mg/kg to 9 mg/kg, 2.3 mg/kg to 9 mg/kg, 2.4 mg/kg to 9 mg/kg, 2.5 mg/kg to 9 mg/kg, 2.6 mg/kg to 9 mg/kg, 2.7 mg/kg to 9 mg/kg, 2.8 mg/kg to 9 mg/kg, 2.9 mg/kg to 9 mg/kg, 3 mg/kg to 9 mg/kg, 3.1 mg/kg to 9 mg/kg, 3.2 mg/kg to 9 mg/kg, 3.3 mg/kg to 9 mg/kg, 3.4 mg/kg to 9 mg/kg, 3.5 mg/kg to 9 mg/kg, 3.6 mg/kg to 9 mg/kg, 3.7 mg/kg to 9 mg/kg, 3.8 mg/kg to 9 mg/kg, 3.9 mg/kg to 9 mg/kg, 4 mg/kg to 9 mg/kg, 4.5 mg/kg to 9 mg/kg, 5 mg/kg to 9 mg/kg, 5.5 mg/kg to 9 mg/kg, 6 mg/kg to 9 mg/kg, 6.5 mg/kg to 9 mg/kg, 7 mg/kg to 9 mg/kg, 7.2 mg/kg to 9 mg/kg, or 8 mg/kg to 9/mg/kg of at least one bispecific anti-Abeta/TfR antibody is administered to the subject relative to the body weight of the subject.

In certain embodiments, 1 mg/kg to 7.2 mg/kg, 1.2 mg/kg to 7.2 mg/kg, 1.4 mg/kg to 7.2 mg/kg, 1.8 mg/kg to 7.2 mg/kg, 1.9 mg/kg to 7.2 mg/kg, 2 mg/kg to 7.2 mg/kg, 2.1 mg/kg to 7.2 mg/kg, 2.2 mg/kg to 7.2 mg/kg, 2.3 mg/kg to 7.2 mg/kg, 2.4 mg/kg to 7.2 mg/kg, 2.5 mg/kg to 7.2 mg/kg, 2.6 mg/kg to 7.2 mg/kg, 2.7 mg/kg to 7.2 mg/kg, 2.8 mg/kg to 7.2 mg/kg, 2.9 mg/kg to 7.2 mg/kg, 3 mg/kg to 7.2 mg/kg, 3.1 mg/kg to 7.2 mg/kg, 3.2 mg/kg to 7.2 mg/kg, 3.3 mg/kg to 7.2 mg/kg, 3.4 mg/kg to 7.2 mg/kg, 3.5 mg/kg to 7.2 mg/kg, 3.6 mg/kg to 7.2 mg/kg, 3.7 mg/kg to 7.2 mg/kg, 3.8 mg/kg to 7.2 mg/kg, 3.9 mg/kg to 7.2 mg/kg, 4 mg/kg to 7.2 mg/kg, 4.5 mg/kg to 7.2 mg/kg, 5 mg/kg to 7.2 mg/kg, 5.5 mg/kg to 7.2 mg/kg, 6 mg/kg to 7.2 mg/kg, 6.5 mg/kg to 7.2 mg/kg, or 7 mg/kg to 7.2 mg/kg of at least one bispecific anti-Abeta/TfR antibody is administered to the subject relative to the body weight of the subject.

In certain embodiments, 1 mg/kg to 6.5 mg/kg, 1.2 mg/kg to 6.5 mg/kg, 1.4 mg/kg to 6.5 mg/kg, 1.8 mg/kg to 6.5 mg/kg, 1.9 mg/kg to 6.5 mg/kg, 2 mg/kg to 6.5 mg/kg, 2.1 mg/kg to 6.5 mg/kg, 2.2 mg/kg to 6.5 mg/kg, 2.3 mg/kg to 6.5 mg/kg, 2.4 mg/kg to 6.5 mg/kg, 2.5 mg/kg to 6.5 mg/kg, 2.6 mg/kg to 6.5 mg/kg, 2.7 mg/kg to 6.5 mg/kg, 2.8 mg/kg to 6.5 mg/kg, 2.9 mg/kg to 6.5 mg/kg, 3 mg/kg to 6.5 mg/kg, 3.1 mg/kg to 6.5 mg/kg, 3.2 mg/kg to 6.5 mg/kg, 3.3 mg/kg to 6.5 mg/kg, 3.4 mg/kg to 6.5 mg/kg, 3.5 mg/kg to 6.5 mg/kg, 3.6 mg/kg to 6.5 mg/kg, 3.7 mg/kg to 6.5 mg/kg, 3.8 mg/kg to 6.5 mg/kg, 3.9 mg/kg to 6.5 mg/kg, 4 mg/kg to 6.5 mg/kg, 4.5 mg/kg to 6.5 mg/kg, 5 mg/kg to 6.5 mg/kg, 5.5 mg/kg to 6.5 mg/kg, or 6 mg/kg to 7.2 mg/kg of at least one bispecific anti-Abeta/TfR antibody is administered to the subject relative to the body weight of the subject.

In certain embodiments, 1 mg/kg to 6 mg/kg, 1.2 mg/kg to 6 mg/kg, 1.4 mg/kg to 6 mg/kg, 1.8 mg/kg to 6 mg/kg, 1.9 mg/kg to 6 mg/kg, 2 mg/kg to 6 mg/kg, 2.1 mg/kg to 6 mg/kg, 2.2 mg/kg to 6 mg/kg, 2.3 mg/kg to 6 mg/kg, 2.4 mg/kg to 6 mg/kg, 2.5 mg/kg to 6 mg/kg, 2.6 mg/kg to 6 mg/kg, 2.7 mg/kg to 6 mg/kg, 2.8 mg/kg to 6 mg/kg, 2.9 mg/kg to 6 mg/kg, 3 mg/kg to 6 mg/kg, 3.1 mg/kg to 6 mg/kg, 3.2 mg/kg to 6 mg/kg, 3.3 mg/kg to 6 mg/kg, 3.4 mg/kg to 6 mg/kg, 3.5 mg/kg to 6 mg/kg, 3.6 mg/kg to 6 mg/kg, 3.7 mg/kg to 6 mg/kg, 3.8 mg/kg to 6 mg/kg, 3.9 mg/kg to 6 mg/kg, 4 mg/kg to 6 mg/kg, 4.5 mg/kg to 6 mg/kg, 5 mg/kg to 6 mg/kg, or 5.5 mg/kg to 6 mg/kg of at least one bispecific anti-Abeta/TfR antibody is administered to the subject relative to the body weight of the subject.

In certain embodiments, 1 mg/kg to 5.5 mg/kg, 1.2 mg/kg to 5.5 mg/kg, 1.4 mg/kg to 5.5 mg/kg, 1.8 mg/kg to 5.5 mg/kg, 1.9 mg/kg to 5.5 mg/kg, 2 mg/kg to 5.5 mg/kg, 2.1 mg/kg to 5.5 mg/kg, 2.2 mg/kg to 5.5 mg/kg, 2.3 mg/kg to 5.5 mg/kg, 2.4 mg/kg to 5.5 mg/kg, 2.5 mg/kg to 5.5 mg/kg, 2.6 mg/kg to 5.5 mg/kg, 2.7 mg/kg to 5.5 mg/kg, 2.8 mg/kg to 5.5 mg/kg, 2.9 mg/kg to 5.5 mg/kg, 3 mg/kg to 5.5 mg/kg, 3.1 mg/kg to 5.5 mg/kg, 3.2 mg/kg to 5.5 mg/kg, 3.3 mg/kg to 5.5 mg/kg, 3.4 mg/kg to 5.5 mg/kg, 3.5 mg/kg to 5.5 mg/kg, 3.6 mg/kg to 5.5 mg/kg, 3.7 mg/kg to 5.5 mg/kg, 3.8 mg/kg to 5.5 mg/kg, 3.9 mg/kg to 5.5 mg/kg, 4 mg/kg to 5.5 mg/kg, 4.5 mg/kg to 5.5 mg/kg, 5 mg/kg to 5.5 mg/kg of at least one bispecific anti-Abeta/TfR antibody is administered to the subject relative to the body weight of the subject.

In certain embodiments, 1 mg/kg to 5 mg/kg, 1.2 mg/kg to 5 mg/kg, 1.4 mg/kg to 5 mg/kg, 1.8 mg/kg to 5 mg/kg, 1.9 mg/kg to 5 mg/kg, 2 mg/kg to 5 mg/kg, 2.1 mg/kg to 5 mg/kg, 2.2 mg/kg to 5 mg/kg, 2.3 mg/kg to 5 mg/kg, 2.4 mg/kg to 5 mg/kg, 2.5 mg/kg to 5 mg/kg, 2.6 mg/kg to 5 mg/kg, 2.7 mg/kg to 5 mg/kg, 2.8 mg/kg to 5 mg/kg, 2.9 mg/kg to 5 mg/kg, 3 mg/kg to 5 mg/kg, 3.1 mg/kg to 5 mg/kg, 3.2 mg/kg to 5 mg/kg, 3.3 mg/kg to 5 mg/kg, 3.4 mg/kg to 5 mg/kg, 3.5 mg/kg to 5 mg/kg, 3.6 mg/kg to 5 mg/kg, 3.7 mg/kg to 5 mg/kg, 3.8 mg/kg to 5 mg/kg, 3.9 mg/kg to 5 mg/kg, 4 mg/kg to 5 mg/kg, 4.5 mg/kg to 5 mg/kg of at least one bispecific anti-Abeta/TfR antibody is administered to the subject relative to the body weight of the subject.

In certain embodiments, 1 mg/kg to 4.6 mg/kg, 1.2 mg/kg to 4.6 mg/kg, 1.4 mg/kg to 4.6 mg/kg, 1.8 mg/kg to 4.6 mg/kg, 1.9 mg/kg to 4.6 mg/kg, 2 mg/kg to 4.6 mg/kg, 2.1 mg/kg to 4.6 mg/kg, 2.2 mg/kg to 4.6 mg/kg, 2.3 mg/kg to 4.6 mg/kg, 2.4 mg/kg to 4.6 mg/kg, 2.5 mg/kg to 4.6 mg/kg, 2.6 mg/kg to 4.6 mg/kg, 2.7 mg/kg to 4.6 mg/kg, 2.8 mg/kg to 4.6 mg/kg, 2.9 mg/kg to 4.6 mg/kg, 3 mg/kg to 4.6 mg/kg, 3.1 mg/kg to 4.6 mg/kg, 3.2 mg/kg to 4.6 mg/kg, 3.3 mg/kg to 4.6 mg/kg, 3.4 mg/kg to 4.6 mg/kg, 3.5 mg/kg to 4.6 mg/kg, 3.6 mg/kg to 4.6 mg/kg, 3.7 mg/kg to 4.6 mg/kg, 3.8 mg/kg to 4.6 mg/kg, 3.9 mg/kg to 4.6 mg/kg, 4 mg/kg to 4.6 mg/kg, 4.5 mg/kg to 4.6 mg/kg of at least one bispecific anti-Abeta/TfR antibody is administered to the subject relative to the body weight of the subject.

In certain embodiments, 1 mg/kg to 4.2 mg/kg, 1.2 mg/kg to 4.2 mg/kg, 1.4 mg/kg to 4.2 mg/kg, 1.8 mg/kg to 4.2 mg/kg, 1.9 mg/kg to 4.2 mg/kg, 2 mg/kg to 4.2 mg/kg, 2.1 mg/kg to 4.2 mg/kg, 2.2 mg/kg to 4.2 mg/kg, 2.3 mg/kg to 4.2 mg/kg, 2.4 mg/kg to 4.2 mg/kg, 2.5 mg/kg to 4.2 mg/kg, 2.6 mg/kg to 4.2 mg/kg, 2.7 mg/kg to 4.2 mg/kg, 2.8 mg/kg to 4.2 mg/kg, 2.9 mg/kg to 4.2 mg/kg, 3 mg/kg to 4.2 mg/kg, 3.1 mg/kg to 4.2 mg/kg, 3.2 mg/kg to 4.2 mg/kg, 3.3 mg/kg to 4.2 mg/kg, 3.4 mg/kg to 4.2 mg/kg, 3.5 mg/kg to 4.2 mg/kg, 3.6 mg/kg to 4.2 mg/kg, 3.7 mg/kg to 4.2 mg/kg, 3.8 mg/kg to 4.2 mg/kg, 3.9 mg/kg to 4.2 mg/kg, 4 mg/kg to 4.2 mg/kg of at least one bispecific anti-Abeta/TfR antibody is administered to the subject relative to the body weight of the subject.

In certain embodiments, 1 mg/kg to 3.8 mg/kg, 1.2 mg/kg to 3.8 mg/kg, 1.4 mg/kg to 3.8 mg/kg, 1.8 mg/kg to 3.8 mg/kg, 1.9 mg/kg to 3.8 mg/kg, 2 mg/kg to 3.8 mg/kg, 2.1 mg/kg to 3.8 mg/kg, 2.2 mg/kg to 3.8 mg/kg, 2.3 mg/kg to 3.8 mg/kg, 2.4 mg/kg to 3.8 mg/kg, 2.5 mg/kg to 3.8 mg/kg, 2.6 mg/kg to 3.8 mg/kg, 2.7 mg/kg to 3.8 mg/kg, 2.8 mg/kg to 3.8 mg/kg, 2.9 mg/kg to 3.8 mg/kg, 3 mg/kg to 3.8 mg/kg, 3.1 mg/kg to 3.8 mg/kg, 3.2 mg/kg to 3.8 mg/kg, 3.3 mg/kg to 3.8 mg/kg, 3.4 mg/kg to 3.8 mg/kg, 3.5 mg/kg to 3.8 mg/kg, 3.6 mg/kg to 3.8 mg/kg, 3.7 mg/kg to 3.8 mg/kg of at least one bispecific anti-Abeta/TfR antibody is administered to the subject relative to the body weight of the subject.

In certain embodiments, 1 mg/kg to 3.6 mg/kg, 1.2 mg/kg to 3.6 mg/kg, 1.4 mg/kg to 3.6 mg/kg, 1.8 mg/kg to 3.6 mg/kg, 1.9 mg/kg to 3.6 mg/kg, 2 mg/kg to 3.6 mg/kg, 2.1 mg/kg to 3.6 mg/kg, 2.2 mg/kg to 3.6 mg/kg, 2.3 mg/kg to 3.6 mg/kg, 2.4 mg/kg to 3.6 mg/kg, 2.5 mg/kg to 3.6 mg/kg, 2.6 mg/kg to 3.6 mg/kg, 2.7 mg/kg to 3.6 mg/kg, 2.8 mg/kg to 3.6 mg/kg, 2.9 mg/kg to 3.6 mg/kg, 3 mg/kg to 3.6 mg/kg, 3.1 mg/kg to 3.6 mg/kg, 3.2 mg/kg to 3.6 mg/kg, 3.3 mg/kg to 3.6 mg/kg, 3.4 mg/kg to 3.6 mg/kg, 3.5 mg/kg to 3.6 mg/kg of at least one bispecific anti-Abeta/TfR antibody is administered to the subject relative to the body weight of the subject.

In certain embodiments, 2.2 mg/kg to 4.2 mg/kg, 2.3 mg/kg to 4.2 mg/kg, 2.4 mg/kg to 4.2 mg/kg, 2.5 mg/kg to 4.2 mg/kg, 2.6 mg/kg to 4.2 mg/kg, 2.7 mg/kg to 4.2 mg/kg, 2.8 mg/kg to 4.2 mg/kg, 2.9 mg/kg to 4.2 mg/kg, 3 mg/kg to 4.2 mg/kg, 3.1 mg/kg to 4.2 mg/kg, 3.2 mg/kg to 4.2 mg/kg, 3.3 mg/kg to 4.2 mg/kg, 3.4 mg/kg to 4.2 mg/kg, 3.5 mg/kg to 4.2 mg/kg, 3.6 mg/kg to 4.2 mg/kg, 3.7 mg/kg to 4.2 mg/kg, 3.8 mg/kg to 4.2 mg/kg, 3.9 mg/kg to 4.2 mg/kg, 4 mg/kg to 4.2 mg/kg, 4.1 mg/kg to 4.2 mg/kg, 2.2 mg/kg to 4.1 mg/kg, 2.2 mg/kg to 4 mg/kg, 2.2 mg/kg to 3.9 mg/kg, 2.2 mg/kg to 3.8 mg/kg, 2.2 mg/kg to 3.7 mg/kg, 2.2 mg/kg to 3.6 mg/kg, 2.2 mg/kg to 3.5 mg/kg, 2.2 mg/kg to 3.4 mg/kg, 2.2 mg/kg to 3.3 mg/kg, 2.2 mg/kg to 3.2 mg/kg, 2.2 mg/kg to 3.1 mg/kg, 2.2 mg/kg to 3 mg/kg, 2.2 mg/kg to 2.9 mg/kg, 2.2 mg/kg to 2.8 mg/kg, 2.2 mg/kg to 2.7 mg/kg, 2.2 mg/kg to 2.6 mg/kg, 2.2 mg/kg to 2.5 mg/kg, 2.2 mg/kg to 2.4 mg/kg, or 2.2 mg/kg to 2.3 of at least one bispecific anti-Abeta/TfR antibody is administered to the subject relative to the body weight of the subject.

In certain embodiments, 2.2 mg/kg to 4.2 mg/kg, 2.3 mg/kg to 4.2 mg/kg, 2.4 mg/kg to 4.2 mg/kg, 2.5 mg/kg to 4.2 mg/kg, 2.6 mg/kg to 4.2 mg/kg, 2.7 mg/kg to 4.2 mg/kg, 2.8 mg/kg to 4.2 mg/kg, 2.9 mg/kg to 4.2 mg/kg, 3 mg/kg to 4.2 mg/kg, 3.1 mg/kg to 4.2 mg/kg, 3.2 mg/kg to 4.2 mg/kg, 3.3 mg/kg to 4.2 mg/kg, 3.4 mg/kg to 4.2 mg/kg, 3.5 mg/kg to 4.2 mg/kg, 3.6 mg/kg to 4.2 mg/kg, 3.7 mg/kg to 4.2 mg/kg, 3.8 mg/kg to 4.2 mg/kg, 3.9 mg/kg to 4.2 mg/kg, 4 mg/kg to 4.2 mg/kg, 4.1 mg/kg to 4.2 mg/kg, 2.6 mg/kg to 4.1 mg/kg, 2.6 mg/kg to 4 mg/kg, 2.6 mg/kg to 3.9 mg/kg, 2.6 mg/kg to 3.8 mg/kg, 2.6 mg/kg to 3.7 mg/kg, 2.6 mg/kg to 3.6 mg/kg, 2.6 mg/kg to 3.5 mg/kg, 2.6 mg/kg to 3.4 mg/kg, 2.6 mg/kg to 3.3 mg/kg, 2.6 mg/kg to 3.2 mg/kg, 2.6 mg/kg to 3.1 mg/kg, 2.6 mg/kg to 3 mg/kg, 2.6 mg/kg to 2.9 mg/kg, 2.6 mg/kg to 2.8 mg/kg, or 2.6 mg/kg to 2.7 mg/kg of at least one bispecific anti-Abeta/TfR antibody is administered to the subject relative to the body weight of the subject.

In certain embodiments, 1.2 mg/kg to 4.2 mg/kg, 1.2 mg/kg to 4.1 mg/kg, 1.2 mg/kg to 4.0 mg/kg, 1.2 mg/kg to 3.9 mg/kg, 1.2 mg/kg to 3.8 mg/kg, 1.2 mg/kg to 3.7 mg/kg, 1.2 mg/kg to 3.6 mg/kg, 1.2 mg/kg to 3.5 mg/kg, 1.2 mg/kg to 3.4 mg/kg, 1.2 mg/kg to 3.3 mg/kg, 1.2 mg/kg to 3.1 mg/kg, 1.2 mg/kg to 3 mg/kg, 1.2 mg/kg to 2.9 mg/kg, 1.2 mg/kg to 2.8 mg/kg, 1.2 mg/kg to 2.7 mg/kg, 1.2 mg/kg to 2.6 mg/kg, 1.2 mg/kg to 2.5 mg/kg, 1.2 mg/kg to 2.4 mg/kg, 1.2 mg/kg to 2.3 mg/kg, 1.2 mg/kg to 2.2 mg/kg, 1.2 mg/kg to 2.1 mg/kg, 1.2 mg/kg to 2 mg/kg, 1.2 mg/kg to 1.9 mg/kg, 1.2 mg/kg to 1.8 mg/kg, 1.3 mg/kg to 2.6 mg/kg, 1.4 mg/kg to 2.5 mg/kg, 1.5 mg/kg to 2.4 mg/kg, 1.6 mg/kg to 2.3 mg/kg, 1.7 mg/kg to 2.2 mg/kg, 1.8 mg/kg to 2.1 mg/kg of at least one bispecific anti-Abeta/TfR antibody is administered to the subject relative to the body weight of the subject.

In certain embodiments, 2.6 mg/kg to 4.2 mg/kg, 2.3 mg/kg to 4.2 mg/kg, 2.4 mg/kg to 4.2 mg/kg, 2.5 mg/kg to 4.2 mg/kg, 2.6 mg/kg to 4.2 mg/kg, 2.7 mg/kg to 4.2 mg/kg, 2.8 mg/kg to 4.2 mg/kg, 2.9 mg/kg to 4.2 mg/kg, 3 mg/kg to 4.2 mg/kg, 3.1 mg/kg to 4.2 mg/kg, 3.2 mg/kg to 4.2 mg/kg, 3.3 mg/kg to 4.2 mg/kg, 3.4 mg/kg to 4.2 mg/kg, 3.5 mg/kg to 4.2 mg/kg, 3.6 mg/kg to 4.2 mg/kg, 3.7 mg/kg to 4.2 mg/kg, 3.8 mg/kg to 4.2 mg/kg, 3.9 mg/kg to 4.2 mg/kg, 4 mg/kg to 4.2 mg/kg, 4.1 mg/kg to 4.2 mg/kg, 2.6 mg/kg to 4.1 mg/kg, 2.6 mg/kg to 4 mg/kg, 2.6 mg/kg to 3.9 mg/kg, 2.6 mg/kg to 3.8 mg/kg, 2.6 mg/kg to 3.7 mg/kg, 2.6 mg/kg to 3.6 mg/kg, 2.6 mg/kg to 3.5 mg/kg, 2.6 mg/kg to 3.4 mg/kg, 2.6 mg/kg to 3.3 mg/kg, 2.6 mg/kg to 3.2 mg/kg, 2.6 mg/kg to 3.1 mg/kg, 2.6 mg/kg to 3 mg/kg, 2.6 mg/kg to 2.9 mg/kg, 2.6 mg/kg to 2.8 mg/kg, or 2.6 mg/kg to 2.7 mg/kg of at least one bispecific anti-Abeta/TfR antibody is administered to the subject relative to the body weight of the subject.

In certain embodiments, 2.6 mg/kg to 3.6 mg/kg, 2.7 mg/kg to 3.6 mg/kg, 2.8 mg/kg to 3.6 mg/kg, 2.9 mg/kg to 3.6 mg/kg, 3 mg/kg to 3.6 mg/kg, 3.1 mg/kg to 3.6 mg/kg, 3.2 mg/kg to 3.6 mg/kg, 3.3 mg/kg to 3.6 mg/kg, 3.4 mg/kg to 3.6 mg/kg, 3.5 mg/kg to 3.6 mg/kg, 2.6 mg/kg to 3.5 mg/kg, 2.6 mg/kg to 3.4 mg/kg, 2.6 mg/kg to 3.3 mg/kg, 2.6 mg/kg to 3.2 mg/kg, 2.6 mg/kg to 3.1 mg/kg, 2.6 mg/kg to 3 mg/kg, 2.6 mg/kg to 2.9 mg/kg, 2.6 mg/kg to 2.8 mg/kg, or 2.6 mg/kg to 2.7 mg/kg of at least one bispecific anti-Abeta/TfR antibody is administered to the subject relative to the body weight of the subject.

In some embodiments, 0.5 mg/kg of at least one bispecific anti-Abeta/TfR antibody is administered to the subject relative to body weight of the subject. In some embodiments, 1 mg/kg of at least one bispecific anti-Abeta/TfR antibody is administered to the subject relative to body weight of the subject. In some embodiments, 1.2 mg/kg of at least one bispecific anti-Abeta/TfR antibody is administered to the subject relative to body weight of the subject. In some embodiments, 1.8 mg/kg of at least one bispecific anti-Abeta/TfR antibody is administered to the subject relative to body weight of the subject. In some embodiments, 2 mg/kg of at least one bispecific anti-Abeta/TfR antibody is administered to the subject relative to body weight of the subject. In some embodiments, 2.6 mg/kg of at least one bispecific anti-Abeta/TfR antibody is administered to the subject relative to body weight of the subject. In some embodiments, 2.8 mg/kg of at least one bispecific anti-Abeta/TfR antibody is administered to the subject relative to body weight of the subject. In some embodiments, 3 mg/kg of at least one bispecific anti-Abeta/TfR antibody is administered to the subject relative to body weight of the subject. In some embodiments, 3.2 mg/kg of at least one bispecific anti-Abeta/TfR antibody is administered to the subject relative to body weight of the subject. In some embodiments, 3.4 mg/kg of at least one bispecific anti-Abeta/TfR antibody is administered to the subject relative to body weight of the subject. In some embodiments, 3.6 mg/kg of at least one bispecific anti-Abeta/TfR antibody is administered to the subject relative to body weight of the subject. In some embodiments, 4 mg/kg of at least one bispecific anti-Abeta/TfR antibody is administered to the subject relative to body weight of the subject. In some embodiments, 4.5 mg/kg of at least one bispecific anti-Abeta/TfR antibody is administered to the subject relative to body weight of the subject. In some embodiments, 5 mg/kg of at least one bispecific anti-Abeta/TfR antibody is administered to the subject relative to body weight of the subject. In some embodiments, 5.5 mg/kg of at least one bispecific anti-Abeta/TfR antibody is administered to the subject relative to body weight of the subject. In some embodiments, 6 mg/kg of at least one bispecific anti-Abeta/TfR antibody is administered to the subject relative to body weight of the subject. In some embodiments, 6.5 mg/kg of at least one bispecific anti-Abeta/TfR antibody is administered to the subject relative to body weight of the subject. In some embodiments, 7 mg/kg of at least one bispecific anti-Abeta/TfR antibody is administered to the subject relative to body weight of the subject. In some embodiments, 7.2 mg/kg of at least one bispecific anti-Abeta/TfR antibody is administered to the subject relative to body weight of the subject. In some embodiments, 8 mg/kg of at least one bispecific anti-Abeta/TfR antibody is administered to the subject relative to body weight of the subject. In some embodiments, 9 mg/kg of at least one bispecific anti-Abeta/TfR antibody is administered to the subject relative to body weight of the subject.

In certain embodiments, a first dose of 1 mg/kg to 7.2 mg/kg, 1.2 mg/kg to 7.2 mg/kg, 1.4 mg/kg to 7.2 mg/kg, 1.8 mg/kg to 7.2 mg/kg, 1.9 mg/kg to 7.2 mg/kg, 2 mg/kg to 7.2 mg/kg, 2.1 mg/kg to 7.2 mg/kg, 2.2 mg/kg to 7.2 mg/kg, 2.3 mg/kg to 7.2 mg/kg, 2.4 mg/kg to 7.2 mg/kg, 2.5 mg/kg to 7.2 mg/kg, 2.6 mg/kg to 7.2 mg/kg, 2.7 mg/kg to 7.2 mg/kg, 2.8 mg/kg to 7.2 mg/kg, 2.9 mg/kg to 7.2 mg/kg, 3 mg/kg to 7.2 mg/kg, 3.1 mg/kg to 7.2 mg/kg, 3.2 mg/kg to 7.2 mg/kg, 3.3 mg/kg to 7.2 mg/kg, 3.4 mg/kg to 7.2 mg/kg, 3.5 mg/kg to 7.2 mg/kg, 3.6 mg/kg to 7.2 mg/kg, 3.7 mg/kg to 7.2 mg/kg, 3.8 mg/kg to 7.2 mg/kg, 3.9 mg/kg to 7.2 mg/kg, 4 mg/kg to 7.2 mg/kg, 4.5 mg/kg to 7.2 mg/kg, 5 mg/kg to 7.2 mg/kg, 5.5 mg/kg to 7.2 mg/kg, 6 mg/kg to 7.2 mg/kg, 6.5 mg/kg to 7.2 mg/kg, or 7 mg/kg to 7.2 mg/kg relative to the body weight of the subject of the at least one bispecific anti-Abeta/TfR antibody is administered to the subject for a number of administrations and after said number of administrations administration is continued with a lower second dose of 0.2 mg/kg to 3.6 mg/kg, 0.3 mg/kg to 3.6 mg/kg, 0.4 mg/kg to 3.6 mg/kg, 0.5 mg/kg to 3.6 mg/kg, 0.6 mg/kg to 3.6 mg/kg, 0.7 mg/kg to 3.6 mg/kg, 0.8 mg/kg to 3.6 mg/kg, 0.9 mg/kg to 3.6 mg/kg, 1.0 mg/kg to 1.1 mg/kg, 1.2 mg/kg to 3.6 mg/kg, 1.3 mg/kg to 3.6 mg/kg, 1.4 mg/kg to 3.6 mg/kg, 1.5 mg/kg to 3.6 mg/kg, 1.6 mg/kg to 3.6 mg/kg, 1.7 mg/kg to 3.6 mg/kg, 1.8 mg/kg to 3.6 mg/kg, 1.9 mg/kg to 3.6 mg/kg, 2 mg/kg to 3.6 mg/kg, 2.1 mg/kg to 3.6 mg/kg, 2.2 mg/kg to 3.6 mg/kg, 2.3 mg/kg to 3.6 mg/kg, 2.4 mg/kg to 3.6 mg/kg, 2.5 mg/kg to 3.6 mg/kg, 2.6 mg/kg to 3.6 mg/kg, 2.7 mg/kg to 3.6 mg/kg, 2.8 mg/kg to 3.6 mg/kg, 2.9 mg/kg to 3.6 mg/kg, 3 mg/kg to 3.6 mg/kg, 3.1 mg/kg to 3.6 mg/kg, 3.2 mg/kg to 3.6 mg/kg, 3.3 mg/kg to 3.6 mg/kg, 3.4 mg/kg to 3.6 mg/kg, 3.5 mg/kg to 3.6 mg/kg, 2.6 mg/kg to 3.5 mg/kg, 2.6 mg/kg to 3.4 mg/kg, 2.6 mg/kg to 3.3 mg/kg, 2.6 mg/kg to 3.2 mg/kg, 2.6 mg/kg to 3.1 mg/kg, 2.6 mg/kg to 3 mg/kg, 2.6 mg/kg to 2.9 mg/kg, 2.6 mg/kg to 2.8 mg/kg, or 2.6 mg/kg to 2.7 mg/kg relative to the body weight of the subject of the at least one bispecific anti-Abeta/TfR antibody.

In certain embodiments, the number of administrations is 3 to 60. In certain embodiments the number of administrations is 3, 6, 12, 18, 24, 36, 48 or 60.

In one preferred embodiment, the at least one bispecific anti-Abeta/TfR antibody is trontinemab (RG6102).

### Dosing Regimens for the at Least One Bispecific Anti-Abeta/TfR Antibody

The methods of the present invention, in certain embodiments, comprise administering to a subject a composition comprising a therapeutically effective amount of at least one bispecific anti-Abeta/TfR antibody. One of ordinary skill in the art will understand that any of the therapeutically effective amounts of the at least one bispecific anti-Abeta/TfR antibody may be administered one or more times according to one or more dosing regimens. One of ordinary skill in the art will be able to determine, depending upon a number of factors including pharmacodynamic characteristics, route of administration, dose, and health, age, and weight of the subject to be treated and, with the information disclosed herein, the appropriate dosing regimen(s) for each subject.

In some embodiments, a composition comprising at least one bispecific anti-Abeta/TfR antibody is administered every day, every other day, every third day, once every week, once every two weeks (“biweekly”), once every four weeks (“four-week interval”), once every month, once every six weeks, once every eight weeks, once every two months, once every ten weeks, once every twelve weeks, once every three months, once every fourteen weeks, once every sixteen weeks, once every four months, once every eighteen weeks, once every twenty weeks, once every five months, once every 22 weeks, once every 24 weeks, once every six months, once every eight months, once every seven months, once every eight months, once every nine months, once every ten months, once every eleven months, once every twelve months, once every thirteen months, once every thirteen months, once every fourteen months, once every fifteen months, once every sixteen months, once every seventeen months, or once every eighteen months. In some embodiments, a composition comprising at least one bispecific anti-Abeta/TfR antibody is administered every day, every other day, every third day, once every week, once every two weeks (“biweekly”), once every four weeks (“four-week interval”), or once every month. In some embodiments, a composition comprising a therapeutically effective amount of at least one bispecific anti-Abeta/TfR antibody is administered once every two weeks or once every four weeks. In some embodiments, a composition comprising a therapeutically effective amount of at least one bispecific anti-Abeta/TfR antibody is administered once every two weeks. In one preferred embodiment, a composition comprising a therapeutically effective amount of at least one bispecific anti-Abeta/TfR antibody is administered once every four weeks. In a further preferred embodiment, a composition comprising a therapeutically effective amount of at least one bispecific anti-Abeta/TfR antibody is administered once every month.

In some embodiments, a composition comprising a therapeutically effective amount of trontinemab is administered once every week. In some embodiments, a composition comprising a therapeutically effective amount of trontinemab is administered once every two weeks. In some embodiments, a composition comprising a therapeutically effective amount of trontinemab is administered once every three weeks. In one preferred embodiment, a composition comprising a therapeutically effective amount of trontinemab is administered once every four weeks. In a further preferred embodiment, a composition comprising a therapeutically effective amount of trontinemab is administered once every month.

In some embodiments, a composition comprising 1.8 mg/kg, 2.6 mg/kg, 3 mg/kg, 3.6 mg/kg or 7.2 mg/kg of at least one bispecific anti-Abeta/TfR antibody relative to the body weight of the subject is administered to the subject once every week. In some embodiments, a composition comprising 1.8 mg/kg, 2.6 mg/kg, 3 mg/kg, 3.6 mg/kg or 7.2 mg/kg of at least one bispecific anti-Abeta/TfR antibody relative to body weight of the subject is administered to the subject once every two weeks. In some embodiments, a composition comprising 1.8 mg/kg, 2.6 mg/kg, 3 mg/kg, 3.6 mg/kg or 7.2 mg/kg of at least one bispecific anti-Abeta/TfR antibody relative to body weight of the subject is administered to the subject once every three weeks. In one preferred embodiment, a composition comprising 1.8 mg/kg, 2.6 mg/kg, 3 mg/kg, 3.6 mg/kg or 7.2 mg/kg of at least one bispecific anti-Abeta/TfR antibody relative to body weight of the subject is administered to the subject once every four weeks. In a further preferred embodiment, a composition comprising 1.8 mg/kg, 2.6 mg/kg, 3 mg/kg, 3.6 mg/kg or 7.2 mg/kg of at least one bispecific anti-Abeta/TfR antibody relative to body weight of the subject is administered to the subject once every month.

In some embodiments, a composition comprising 1.8 to 7.2 mg/kg of at least one bispecific anti-Abeta/TfR antibody relative to body weight of the subject is administered to the subject once every four weeks. In one preferred embodiment, a composition comprising 2.6 to 3.6 mg/kg of at least one bispecific anti-Abeta/TfR antibody relative to body weight of the subject is administered to the subject once every four weeks or every month.

In some embodiments, a composition comprising 1.8 to 7.2 mg/kg of trontinemab relative to body weight of the subject is administered to the subject once every four weeks. In one preferred embodiment, a composition comprising 2.6 to 3.6 mg/kg of trontinemab relative to body weight of the subject is administered to the subject once every four weeks or every month.

### Composition Comprising the at Least One Bispecific Anti-Abeta/TfR Antibody

In some embodiments, the at least one bispecific anti-Abeta/TfR antibody is comprised in a composition. In some embodiments, the composition consists of at least one bispecific anti-Abeta/TfR antibody. In some embodiments, the composition comprises at least one bispecific anti-Abeta/TfR antibody and further comprises at least one additional component. The at least one additional component can be chosen from suitable physiologically acceptable excipients for human use.

The compositions of the present invention may be in the form of a solution, lyophilizate and/or any other suitable form deemed appropriate by one of ordinary skill in the art. The route of administration of the compositions of the present invention may be any suitable route, including intravenous and subcutaneous administration. In some embodiments, the composition is formulated as a sterile, non-pyrogenic liquid for intravenous administration. In some embodiments, the composition is a saline solution.

Formulations for anti-Abeta antibodies are known from the art, e.g., as disclosed in WO 2008/071394, WO 2013/131866, CN 115227813, JP 2014/001232, WO 2009/017467, and WO 2006/083689.

Generally, the anti-Abeta antibody used according to the current invention can be formulated in any formulation suitable for intravenous application. For example, but not by way of limitation, formulations known for other antibodies, especially other anti-Abeta antibodies, can be used.

In certain embodiments, the anti-Abeta antibody used according to the current invention is formulated in a stable formulation comprising:
about 1 to about 250 mg/mL of the anti-Abeta antibody (w/v);
about 0.001% to about 1% of at least one surfactant (w/v);
about 1 mM to about 100 mM of a buffer;
optionally about 10 mM to about 500 mM of a stabilizer and/or about 5 mM to about 500 mM of a tonicity agent;
at a pH of about 4.0 to about 7.0.

In certain embodiments, the formulation is a liquid formulation.

In certain preferred embodiments, the liquid formulation comprises:
1)
   about 1 to about 200 mg/mL of the anti-Abeta antibody,
   about 0.04% polysorbate 20 (w/v),
   about 20 mM L-histidine,
   about 250 mM sucrose,
   at a pH of about 5.5; or
2)
   about 37.5 mg/mL of the anti-Abeta antibody,
   about 0.02% polysorbate 20 (w/v),
   about 10 mM L-histidine,
   about 125 mM sucrose,
   at a pH of about 5.5; or
3)
   about 37.5 mg/mL of the anti-Abeta antibody,
   about 0.01% polysorbate 20 (w/v),
   about 10 mM L-histidine,
   about 125 mM sucrose,
   at a pH value of about 5.5; or
4)
   about 7.5 mg/mL of the anti-Abeta antibody,
   about 0.04% polysorbate 20 (w/v),
   about 20 mM L-histidine,
   about 250 mM sucrose,
   at a pH value of about 5.5; or
5)
   about 7.5 mg/mL of the anti-Abeta antibody,
   about 0.02% polysorbate 20 (w/v),
   about 10 mM L-histidine,
   about 125 mM sucrose,
   at a pH value of about 5.5; or
6)
   about 37.5 mg/mL Abeta antibody,
   about 0.02% polysorbate 20 (w/v),
   about 10 mM L-histidine,
   about 125 mM trehalose,
   at a pH value of about 5.5; or
7)
   about 37.5 mg/mL of the anti-Abeta antibody,
   about 0.01% polysorbate 20 (w/v),
   about 10 mM L-histidine,
   about 125 mM trehalose,
   at a pH value of about 5.5, or
8)
   about 75 mg/mL of the anti-Abeta antibody,
   about 0.02% polysorbate 20 (w/v),
   about 20 mM L-histidine,
   about 250 mM trehalose,
   at a pH value of about 5.5, or
9)
   about 75 mg/mL of the anti-Abeta antibody,
   about 0.02% polysorbate 20 (w/v),
   about 20 mM L-histidine,
   about 250 mM mannitol,
   at a pH value of about 5.5, or
10)
   about 75 mg/mL of the anti-Abeta antibody,
   about 0.02% polysorbate 20 (w/v),
   about 20 mM L-histidine,
   about 140 mM sodium chloride,
   at a pH value of about 5.5, or
11)
   about 150 mg/mL of the anti-Abeta antibody,
   0.02% polysorbate 20 (w/v),
   about 20 mM L-histidine,
   about 250 mM trehalose,
   at a pH value of about 5.5, or
12)
   about 150 mg/mL of the anti-Abeta antibody,
   about 0.02% polysorbate 20 (w/v),
   about 20 mM L-histidine,
   about 250 mM mannitol,
   at a pH value of about 5.5, or
13)
   about 150 mg/mL of the anti-Abeta antibody,
   about 0.02% polysorbate 20 (w/v),
   about 20 mM L-histidine,
   about 140 mM sodium chloride,
   at a pH value of about 5.5; or
14)
   about 10 mg/mL of the anti-Abeta antibody,
   about 0.01% polysorbate 20 (w/v),
   about 20 mM L-histidine,
   about 140 mM sodium chloride,
   at a pH value of about 5.5.

In certain preferred embodiments, the liquid formulation comprises:
about 1 mg/mL to about 200 mg/mL of the anti-Abeta antibody,
about 0.01% to about 0.1% of a poloxamer (w/v),
about 5 mM to about 50 mM of a buffer,
about 100 mM to about 300 mM of a stabilizer,
at a pH value in the range and including pH 4.5 to pH 7.0.

In certain embodiments, the antibody has a concentration of about 100 mg/ml to about 200 mg/ml, preferably about 150 mg/ml.

In certain embodiments, the poloxamer is present in a concentration of 0.02%-0.06%, preferably in a concentration of about 0.04%. In one preferred embodiment the poloxamer is poloxamer 188.

In certain embodiments, the buffer is a sodium acetate buffer or a Histidine buffer. In one preferred embodiment, the buffer is a Histidine/Histidine-HCl buffer.

In certain embodiments, the buffer has a concentration of about 10 mM to about 30 mM. In one preferred embodiment, the buffer has a concentration of about 20 mM.

In certain embodiments, the formulation has a pH value in the range and including pH 5 to pH 6. In one preferred embodiment, the pH of the formulation is 5.5.

In certain embodiments, the stabilizer is selected from sugars and amino acids.

In certain embodiments, the stabilizer is selected from trehalose and arginine.

In one certain embodiments, the stabilizer is trehalose and has a concentration of about 150 mM to about 250 mM. In one preferred embodiment, the stabilizer is trehalose and has a concentration of about 200 mM.

In certain embodiments, the stabilizer is arginine and has a concentration of about 100 mM to about 150 mM. In one preferred embodiment, the stabilizer is arginine and has a concentration of about 135 mM.

In one aspect, the antibody used in the current invention is within a pharmaceutical composition comprising the anti-Abeta antibody and a buffer, wherein the buffer is a histidine salt buffer or an acetate buffer.

In certain embodiments, the buffer is at a concentration in the range of and including about 5 mM to about 100 mM. In certain embodiments, the buffer is at a concentration in the range of and including about 30 mM to about 70 mM. In one preferred embodiment, the buffer is at a concentration of about 50 mM.

In one preferred embodiment, the buffer is a histidine-acetate buffer or an acetic acid-sodium acetate buffer.

In certain embodiments, the pH value of the pharmaceutical composition is in the range of and including about pH 4.5 to about pH 6.0. In certain embodiments, the pH value of the pharmaceutical composition is in the range of and including about pH 4.7 to about pH 5.5. In one preferred embodiments, the pH value of the pharmaceutical composition is about pH 5.0 or about pH 5.5.

In certain embodiments, the concentration of the anti-Abeta antibody is from about 60 mg/mL to about 200 mg/mL. In certain embodiments, the concentration of the anti-Abeta antibody is from about 90 mg/mL to about 150 mg/mL. In one preferred embodiment, the concentration of the anti-Abeta antibody is about 100 mg/mL or about 150 mg/mL In certain embodiments, the pharmaceutical composition further comprises a surfactant.

In certain embodiments, the surfactant is polysorbate. In one preferred embodiment, the surfactant is polysorbate 80 or polysorbate 20.

In certain embodiments, the concentration of the surfactant is in the range of and including about 0.01% to about 0.1% (w/v). In certain embodiments, the concentration of the surfactant is in the range of and including about 0.02% to about 0.08% (w/v). In one preferred embodiment, the concentration of the surfactant is in the range of and including about 0.04% to 0.06% (w/v).

In certain embodiments, the pharmaceutical composition further comprises a sugar.

In certain embodiments, the pharmaceutical composition comprises sucrose or trehalose. In one preferred embodiment, the pharmaceutical composition comprises sucrose.

In certain embodiments, the pharmaceutical composition comprises a sugar at a concentration in the range of and including about 20 mg/mL to about 100 mg/mL. In certain embodiments, the pharmaceutical composition comprises a sugar at a concentration in the range of and including about 30 mg/mL to about 90 mg/mL. In one preferred embodiment, the pharmaceutical composition comprises a sugar at a concentration of about 40 mg/mL or of about 60 mg/mL or of about 70 mg/mL or of about 80 mg/mL.

In certain embodiments, the pharmaceutical composition further comprises a chelating agent.

In certain embodiments, the chelating agent is ethylenediaminetetraacetic acid (EDTA) disodium salt.

In certain embodiments, the chelating agent is at a concentration in the range of and including about 0.01 mg/mL to about 20 mg/mL. In certain embodiments, the chelating agent is at a concentration range of and including about 0.1 mg/mL to about 1 mg/mL. In one embodiment, the chelating agent is at a concentration of about 0.5 mg/mL.

In certain embodiments, the pharmaceutical composition comprising the following components:
(i)
   (a) about 90 mg/mL to about 150 mg/mL of the anti-Abeta antibody,
   (b) about 0.2 mg/mL to about 0.8 mg/mL of polysorbate,
   (c) about 30 mg/mL to about 90 mg/mL of sucrose,
   (d) about 30 mM to about 70 mM of histidine salt buffer or acetate buffer; the pH of the pharmaceutical composition is from about 4.7 to about 5.5; or
(ii)
   (a) about 90 mg/mL to about 150 mg/mL of the anti-Abeta antibody,
   (b) about 0.2 mg/mL to about 0.8 mg/mL of polysorbate,
   (c) about 30 mg/mL to about 90 mg/mL of sucrose,
   (d) about 30 mM to about 70 mM of histidine salt buffer or acetate buffer;
   (e) from about 0.1 mg/mL to about 1 mg/mL of disodium salt of ethylenediaminetetraacetic acid;
   the pH of the pharmaceutical composition is from about 4.7 to about 5.5;
(iii)
   (a) about 100 mg/mL of the anti-Abeta antibody,
   (b) about 0.4 mg/mL of polysorbate 80,
   (c) about 40 mg/mL of sucrose,
   (d) about 50 mM histidine-acetate buffer;
   the pH of the pharmaceutical composition is about 5.0;
(iv)
   (a) about 100 mg/mL of the anti-Abeta antibody,
   (b) about 0.6 mg/mL of polysorbate 80,
   (c) about 60 mg/mL of sucrose,
   (d) about 50 mM of acetic acid-sodium acetate buffer;
   the pH of the pharmaceutical composition is about 5.0;
(v)
   (a) about 150 mg/mL of the anti-Abeta antibody,
   (b) about 0.6 mg/mL of polysorbate 80,
   (c) about 60 mg/mL of sucrose,
   (d) about 50 mM of acetic acid-sodium acetate buffer;
   the pH of the pharmaceutical composition is about 5.0;
(vi)
   (a) about 100 mg/mL of the anti-Abeta antibody,
   (b) about 0.6 mg/mL polysorbate 80,
   (c) about 70 mg/mL sucrose,
   (d) about 50 mM acetic acid-sodium acetate buffer,
   (e) about 0.5 mg/mL ethylenediaminetetraacetic acid disodium salt;
   the pH of the pharmaceutical composition is about 5.5.

In certain aspects, the antibody used in the current invention is in a formulation comprising about 10 mg of the anti-Abeta antibody in about 10 mM histidine, about 10 mM methionine, about 4% mannitol, and about 0.005% (w/v) polysorbate 80 at a pH of about 6.0.

In certain aspects, the antibody used in the current invention is in a formulation comprising about 10 mg to about 250 mg of the anti-Abeta antibody, about 4% mannitol or about 150 mM NaCl, about 5 mM to about 10 mM histidine, and about 10 mM methionine.

In certain aspects, the antibody used in the current invention is in a formulation comprising the anti-Abeta antibody at a concentration in the range of and including about 17 mg/ml to about 23 mg/ml; histidine at a concentration in the range of and including about 5 mM to about 15 mM; mannitol in an amount in the range of and including about 2% w/v to about 6% w/v; methionine at a concentration in the range of and including about 5 mM to about 15 mM, and polysorbate in an amount in the range of and including about 0.001% w/v to about 0.01% w/v, wherein the formulation has a pH of from 5.5 to 6.5.

In one preferred embodiment, the antibody is present at a concentration of about 20 mg/ml.

In one preferred embodiment, histidine is present at a concentration of about 10 mM.

In one preferred embodiment, mannitol is present in an amount of about 4% w/v In one preferred embodiment, methionine is present at a concentration of about 10 mM.

In one preferred embodiment, the polysorbate is present in an amount of 0.005% w/v.

In one preferred embodiment, the pH is about 6.0.

In one preferred embodiment, the histidine is L-histidine, the mannitol is D-mannitol, the methionine is L-methionine, and the polysorbate is polysorbate-80.

### Reduction of Clinical Decline

One aspect of the invention is directed to a method of reducing clinical decline in a subject having early Alzheimer's disease, such as mild, mild-to-prodromal or prodromal Alzheimer's disease. For example, but not by way of limitation, such embodiments, can comprise administering to said subject a composition comprising a therapeutically effective amount of at least one bispecific anti-Abeta/TfR antibody disclosed herein. In some embodiments, the subject having early Alzheimer's disease has been diagnosed as having mild cognitive impairment due to Alzheimer's disease-intermediate likelihood and/or has been diagnosed as having mild Alzheimer's disease dementia.

Any of the bispecific anti-Abeta/TfR antibodies, therapeutically acceptable amounts thereof, dosing regimens therefore, and compositions comprising the same according to the current invention may be used in the method of reducing clinical decline in a subject having early Alzheimer's disease according to the current invention. For example, in some embodiments, a composition comprising 1.8 mg/kg, 2.6 mg/kg, 3 mg/kg, 3.6 mg/kg or 7.2 mg/kg of the at least one bispecific anti-Abeta/TfR antibody such as trontinemab relative to body weight of the subject is administered to the subject once every week, once every two weeks, once every three weeks, once every four weeks, or once every month. In some embodiments, the clinical decline is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, or at least 60% relative to placebo as determined by CDR-SB. In some embodiments, the above-recited reduction in clinical decline is determined after 1 month, 6 months, 12 months, 18 months, 24 months, 36 months and/or 60 months of administration of the composition comprising a therapeutically effective amount of at least one bispecific anti-Abeta/TfR antibody.

In some embodiments, the clinical decline is reduced by 20% to 60% relative to placebo as determined by CDR-SB. In some embodiments, the clinical decline is reduced by 25% to 60% relative to placebo as determined by CDR-SB. In some embodiments, the clinical decline is reduced by 25% to 50% relative to placebo as determined by CDR-SB. In some embodiments, the clinical decline is reduced by at least 20% relative to placebo as determined by CDR-SB. In some embodiments, the clinical decline is reduced by at least 30% relative to placebo as determined by CDR-SB. In some embodiments, the clinical decline is reduced by at least 25%, such as at least 26% or at least 28%, as determined by CDR-SB. In some embodiments, the clinical decline is reduced by at least 30%, such as at least 35% or at least 38%, as determined by CDR-SB. In some embodiments, the above-recited reduction in clinical decline is determined after 1 month, 6 months, 12 months, 18 months, 24 months, 36 months and/or 60 months of administration of the composition comprising a therapeutically effective amount of at least one bispecific anti-Abeta/TfR antibody.

In some embodiments, the clinical decline is reduced by at least 30%, such as at least 35% or at least 40%, relative to placebo as determined by CDR-SB after 6 months of administration of the composition comprising a therapeutically effective amount of at least one bispecific anti-Abeta/TfR antibody. In some embodiments, the clinical decline is reduced by at least 30%, such as at least 35% or at least 45%, relative to placebo as determined by CDR-SB after 12 months of administration of the composition comprising a therapeutically effective amount of at least one bispecific anti-Abeta/TfR antibody. In some embodiments, the clinical decline is reduced by at least 20%, such as at least 25%, relative to placebo as determined by CDR-SB after 18 months of administration of the composition comprising a therapeutically effective amount of at least one bispecific anti-Abeta/TfR antibody. In one preferred embodiment, the composition comprises 1.8 to 7.2 mg/kg of at least one bispecific anti-Abeta/TfR antibody and is administered once every four weeks or once every month. In one preferred embodiment, the at least one bispecific anti-Abeta/TfR antibody is trontinemab.

In some embodiments, the clinical decline is reduced by at least 1%, at least 2%, at least 3%, at least 4%, at least 5%, at least 6%, at least 7%, at least 8%, at least 9%, at least 10%, at least 11%, at least 12%, at least 13%, or at least 14% relative to placebo as determined by CDR-SB, wherein the subject has been diagnosed as having mild cognitive impairment due to Alzheimer's disease-intermediate likelihood. In some embodiments, the above-recited reduction in clinical decline is determined after 1 month, 6 months, 12 months, 18 months, 24 months, 36 months and/or 60 months of administration of the composition comprising a therapeutically effective amount of at least one bispecific anti-Abeta/TfR antibody.

In some embodiments, the clinical decline is reduced by 10% to 20% relative to placebo as determined by CDR-SB, wherein the subject has been diagnosed as having mild cognitive impairment due to Alzheimer's disease-intermediate likelihood. In some embodiments, the clinical decline is reduced by at least 5%, such as by at 10%, at least 12%, or at least 14%, relative to placebo as determined by CDR-SB, wherein the subject has been diagnosed as having mild cognitive impairment due to Alzheimer's disease-intermediate likelihood. In some embodiments, the above-recited reduction in clinical decline is determined after 1 month, 6 months, 12 months, 18 months, 24 months, 36 months and/or 60 months of administration of the composition comprising a therapeutically effective amount of at least one bispecific anti-Abeta/TfR antibody.

In some embodiments, the clinical decline is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, or at least 51% relative to placebo as determined by CDR-SB, wherein the subject has been diagnosed as having mild Alzheimer's disease dementia. In some embodiments, the above-recited reduction in clinical decline is determined after 1 month, 6 months, 12 months, 18 months, 24 months, 36 months and/or 60 months of administration of the composition comprising a therapeutically effective amount of at least one bispecific anti-Abeta/TfR antibody.

In some embodiments, the clinical decline is reduced by 40% to 60% relative to placebo as determined by CDR-SB, wherein the subject has been diagnosed as having mild Alzheimer's disease dementia. In some embodiments, the clinical decline is reduced by at least 45%, such as by at 48%, at least 50%, or at least 51%, relative to placebo as determined by CDR-SB, wherein the subject has been diagnosed as having mild Alzheimer's disease dementia. In some embodiments, the clinical decline is reduced by at least 51% relative to placebo as determined by CDR-SB, wherein the subject has been diagnosed as having mild Alzheimer's disease dementia. In some embodiments, the above-recited reduction in clinical decline is determined after 1 month, 6 months, 12 months, 18 months, 24 months, 36 months and/or 60 months of administration of the composition comprising a therapeutically effective amount of at least one bispecific anti-Abeta/TfR antibody.

In some embodiments, the clinical decline in the subject diagnosed as having mild Alzheimer's disease dementia is reduced by at least 51% relative to placebo as determined by CDR-SB after 18 months of administration of the composition comprising a therapeutically effective amount of at least one bispecific anti-Abeta/TfR antibody. In some embodiments, the composition comprises 1.8 to 7.2 mg/kg of the at least one bispecific anti-Abeta/TfR antibody and is administered once every two four or once every month. In one preferred embodiment, the at least one bispecific anti-Abeta antibody is trontinemab.

In some embodiments, the reduction in clinical decline is determined after 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 30 months, 36 months, 42 months, 48 months, 54 months, 60 months, 63 months, 66 months, and/or 72 months of administration of the composition comprising a therapeutically effective amount of at least one bispecific anti-Abeta/TfR antibody. In some embodiments, the reduction in clinical decline is determined after 1 month, 6 months, 12 months, 18 months, 24 months, 36 months and/or 60 months of administration of the composition comprising a therapeutically effective amount of at least one bispecific anti-Abeta/TfR antibody.

In some embodiments, the reduction in clinical decline is determined after 1 month of administration of the composition comprising a therapeutically effective amount of at least one bispecific anti-Abeta/TfR antibody. In some embodiments, the reduction in clinical decline is determined after 6 months of administration of the composition comprising a therapeutically effective amount of at least one bispecific anti-Abeta/TfR antibody. In some embodiments, the reduction in clinical decline is determined after 12 months of administration of the composition comprising a therapeutically effective amount of at least one bispecific anti-Abeta/TfR antibody. In some embodiments, the reduction in clinical decline is determined after 18 months of administration of the composition comprising a therapeutically effective amount of at least one bispecific anti-Abeta/TfR antibody. In some embodiments, the reduction in clinical decline is determined after 24 months of administration of the composition comprising a therapeutically effective amount of at least one bispecific anti-Abeta/TfR antibody. In some embodiments, the reduction in clinical decline is determined after 60 months of administration of the composition comprising a therapeutically effective amount of at least one bispecific anti-Abeta/TfR antibody.

In some embodiments, the reduction in clinical decline is determined after administration of a composition comprising a therapeutically effective amount of trontinemab.

In some embodiments, the reduction in clinical decline is determined after 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 30 months, 36 months, 42 months, 48 months, 54 months, 60 months, 63 months, 66 months, and/or 72 months of administration of the composition comprising a therapeutically effective amount of trontinemab. In some embodiments, the reduction in clinical decline is determined after 1 month, 6 months, 12 months, 18 months, 24 months, 36 months, and/or 60 months of administration of the composition comprising a therapeutically effective amount of trontinemab. In some embodiments, the reduction in clinical decline is determined after 1 month of administration of the composition comprising a therapeutically effective amount of trontinemab. In some embodiments, the reduction in clinical decline is determined after 6 months of administration of the composition comprising a therapeutically effective amount of trontinemab. In some embodiments, the reduction in clinical decline is determined after 12 months of administration of the composition comprising a therapeutically effective amount of trontinemab. In some embodiments, the reduction in clinical decline is determined after 18 months of administration of the composition comprising a therapeutically effective amount of trontinemab. In some embodiments, the reduction in clinical decline is determined after 60 months of administration of the composition comprising a therapeutically effective amount of trontinemab. In some embodiments, the reduction in clinical decline is determined after 63 months of administration of the composition comprising a therapeutically effective amount of trontinemab.

Conversion of a Subject from Amyloid Positive to Amyloid Negative

A further aspect according to the invention is a method of converting an amyloid-positive subject to an amyloid-negative subject. In some embodiments, said method comprises administering to said subject a composition comprising at least one bispecific anti-Abeta/TfR antibody disclosed herein. In some embodiments, said subject having early Alzheimer's disease, e.g. mild, mild-to-prodromal or prodromal Alzheimer's disease, has been diagnosed as having mild cognitive impairment due to Alzheimer's disease-intermediate likelihood and/or has been diagnosed as having mild Alzheimer's disease dementia.

Any of the bispecific anti-Abeta/TfR antibodies, therapeutically acceptable amounts thereof, dosing regimens therefore, and compositions comprising the same according to the current invention may be used in the method of converting an amyloid-positive subject to an amyloid-negative subject. For example, in some embodiments, a composition comprising 1.8 mg/kg, 2.6 mg/kg, 3.8 mg/kg, or 7.2 mg/kg of at least one bispecific anti-Abeta/TfR antibody such as trontinemab relative to body weight of the subject is administered to the subject once every week, once every two weeks, once every three weeks, once every four weeks, or once every month.

In some embodiments, administration of the composition results in at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, or at least 81% of the subjects being converted from amyloid positive to amyloid negative, as determined by visual reads of amyloid PET images.

In some embodiments, administration of the composition results in a conversion of 50% to 100%, such as 60% to 90%, of subjects from amyloid positive to amyloid negative, as determined by visual reads of amyloid PET images. In some embodiments, administration of the composition results in at least 55%, such as at least 60% or at least 65%, of the subjects being amyloid negative, as determined by visual reads of amyloid PET images, after 12 months of administration of the composition comprising a therapeutically effective amount of at least one bispecific anti-Abeta/TfR antibody. In some embodiments, administration of the composition results in at least 70%, such as at least 75% or at least 80%, of the subjects being amyloid negative, as determined by visual reads of amyloid PET images, after 18 months of administration of the composition comprising a therapeutically effective amount of at least one bispecific anti-Abeta/TfR antibody. In some embodiments, the composition comprises 1.8 to 7.2 mg/kg of at least one bispecific anti-Abeta/TfR antibody and is administered once every four weeks or once every month. In one preferred embodiment, the at least one bispecific anti-Abeta/TfR antibody is trontinemab.

In some embodiments, administration of the composition results in at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% of the subjects being amyloid negative, as determined by visual reads of amyloid PET images.

In some embodiments, the clinical decline is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, or at least 45% relative to placebo as determined by CDR-SB, wherein the subject is not concomitantly administered at least one Alzheimer's disease medication. In some embodiments, the above-recited reduction in clinical decline is determined after 1 month, 6 months, 12 months, 18 months, 24 months, 36 months, and/or 60 months of administration of the composition comprising a therapeutically effective amount of at least one bispecific anti-Abeta/TfR antibody.

In some embodiments, the clinical decline is reduced by at least 35%, such as by at 40%, at least 42%, or at least 45%, relative to placebo as determined by CDR-SB, wherein the subject is not concomitantly administered at least one Alzheimer's disease medication. In some embodiments, the clinical decline is reduced by at least 45% relative to placebo as determined by CDR-SB, wherein the subject is not concomitantly administered at least one Alzheimer's disease medication. In some embodiments, the above-recited reduction in clinical decline is determined after 1 month, 6 months, 12 months, 18 months, 24 months, 36 months and/or 60 months of administration of the composition comprising a therapeutically effective amount of at least one bispecific anti-Abeta/TfR antibody.

In some embodiments, the clinical decline in the subject who is not concomitantly administered at least one Alzheimer's disease medication is reduced by at least 45% relative to placebo as determined by CDR-SB after 18 months of administration of the composition comprising a therapeutically effective amount of at least one bispecific anti-Abeta/TfR antibody. In some embodiments, the composition comprises 1.8 to 7.2 mg/kg of at least one bispecific anti-Abeta/TfR antibody and is administered once every four weeks or once every month. In one preferred embodiment, the at least one bispecific anti-Abeta/TfR antibody is trontinemab.

### Reduction of Brain Amyloid Level

One aspect according to the current invention is a method of reducing brain amyloid level in a subject in need thereof comprising administering a composition comprising a therapeutically effective amount of at least one bispecific anti-Abeta/TfR antibody according to the current invention and disclosed herein.

One of ordinary skill in the art will understand that, in addition to subjects having Alzheimer's disease, Abeta protein plaque deposits are present in the brains of subjects having other neurodegenerative diseases and conditions and, thus, that the methods according to the current invention will be beneficial for subjects having such neurodegenerative diseases and/or conditions. Such diseases and conditions are known to include, for example, Down's syndrome, chronic traumatic encephalopathy, cerebral amyloid angiopathy, and Lewy Body Dementia. (See, e.g., Catafau et al., “Amyloid PET imaging: applications beyond Alzheimer's disease,” Clin. Transl. Imaging 3(1): 39-55 (2015); and Banerjee, G. et al., “The increasing impact of cerebral amyloid angiopathy: essential new insights for clinical practice,” J. Neurol. Neurosurg. Psychiatry 88: 982-994 (2017).)

In some embodiments, the subject has early Alzheimer's disease.

In one preferred embodiment, the subject has mild, mild-to-prodromal or prodromal Alzheimer's disease.

In some embodiments, the subject has Alzheimer's disease, Down's syndrome, chronic traumatic encephalopathy, cerebral amyloid angiopathy, Lewy Body Dementia, or another brain disease or conditions with Abeta peptide-containing soluble and/or insoluble Abeta aggregates.

In some embodiments, the subject having early Alzheimer's disease has been diagnosed as having mild cognitive impairment due to Alzheimer's disease-intermediate likelihood and/or has been diagnosed as having mild Alzheimer's disease dementia.

Any of the bispecific anti-Abeta/TfR antibodies, therapeutically acceptable amounts thereof, dosing regimens therefore, and compositions comprising the same according to the current invention may be used in the method of reducing brain amyloid level in a subject having early, mild or prodromal Alzheimer's disease. In one preferred embodiment, a composition comprising 1.8 to 7.2 mg/kg, such as, e.g., 1.8 mg/kg, 2.6 mg/kg, 3.8 mg/kg, or 7.2 mg/kg, of at least one bispecific anti-Abeta/TfR antibody such as trontinemab relative to body weight of the subject is administered to the subject once every week, once every two weeks, once every three weeks, once every four weeks, or once every month.

In one preferred embodiment, the at least one bispecific anti-Abeta/TfR antibody is trontinemab.

In some embodiments, the reduction of amyloid in the brain is determined by imaging using binding of radiotracers for brain Abeta protein amyloid and visualized with positron emission tomography (PET). In some embodiments, the reduction in the adjusted mean change from baseline is at least 40 Centiloids (−40 Centiloids from baseline), such as at least 65 Centiloids (−65 Centiloids from baseline) or at least 90 Centiloids (−90 Centiloids from baseline) after 3 months of administration of the composition comprising a therapeutically effective amount of at least one bispecific anti-Abeta/TfR antibody. In some embodiments, the reduction of amyloid in the brain is determined by imaging using binding of radiotracers for brain Abeta protein amyloid and visualized with PET. In some embodiments, the reduction in the adjusted mean change from baseline is at least 50 Centiloids (−50 Centiloids from baseline), such as at least 55 Centiloids (−55 Centiloids from baseline) or at least 60 Centiloids (−60 Centiloids from baseline) after 12 months of administration of the composition comprising a therapeutically effective amount of at least one bispecific anti-Abeta/TfR antibody. In some embodiments, the reduction in the adjusted mean change from baseline is at least 60 Centiloids (−60 Centiloids from baseline), such as at least 65 Centiloids (−65 Centiloids from baseline) or at least 70 Centiloids (−70 Centiloids from baseline) after 18 months of administration of the composition comprising a therapeutically effective amount of at least one bispecific anti-Abeta/TfR antibody.

In some embodiments, said method results in a reduced cerebrospinal fluid Abeta1-42 protein fragment level relative to the cerebrospinal fluid Abeta1-42 protein fragment level prior to said administration. In some embodiments, said method results in a reduction of cerebrospinal fluid Abeta1-42 protein fragment level of at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% relative to the cerebrospinal fluid Abeta1-42 protein fragment level prior to said administration.

In some embodiments, administration of the composition comprising at least one bispecific anti-Abeta/TfR antibody results in at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% of the subjects being amyloid negative, as determined by visual reads of amyloid PET images.

In some embodiments, administration of the composition comprising at least one bispecific anti-Abeta/TfR antibody results in 75% to 100%, such as 80% to 100% or 85% to 100% of the subjects being amyloid negative, as determined by visual reads of amyloid PET images. In some embodiments, administration of the composition comprising at least one bispecific anti-Abeta/TfR antibody results in at least 75%, such as at least 80% or at least 85%, of the subjects being amyloid negative, as determined by visual reads of amyloid PET images. In some embodiments, administration of the composition comprising at least one bispecific anti-Abeta/TfR antibody results in 100% of the subjects being amyloid negative, as determined by visual reads of amyloid PET images.

In some embodiments, at least 30% of the subjects are amyloid negative, as determined by visual reads of amyloid PET images, after 3 months of administration of the composition comprising a therapeutically effective amount of at least one bispecific anti-Abeta/TfR antibody. In some embodiments, at least 30%, such as at least 40%, at least 50%, at least 60%, or at least 70% of the subjects are amyloid negative, as determined by visual reads of amyloid PET images, after 6 months of administration of the composition comprising a therapeutically effective amount of at least one bispecific anti-Abeta/TfR antibody. In some embodiments, at least 75%, such as at least 80% or at least 85%, of the subjects are amyloid negative, as determined by visual reads of amyloid PET images, after 12 months of administration of the composition comprising a therapeutically effective amount of at least one bispecific anti-Abeta/TfR antibody. In some embodiments, at least 75%, such as at least 80%, at least 85%, at least 90%, or at least 95%, of the subjects are amyloid negative, as determined by visual reads of amyloid PET images, after 18 months of administration of the composition comprising a therapeutically effective amount of at least one bispecific anti-Abeta/TfR antibody. In some embodiments, the composition comprises 1.8 to 7.2 mg/kg, such as 1.8 mg/kg, 2.6 mg/kg, 3.6 mg/kg or 7.2 mg/kg, of at least one bispecific anti-Abeta/TfR antibody and is administered once every four weeks or once every month. In one preferred embodiment, the at least one bispecific anti-Abeta/TfR antibody is trontinemab.

In some embodiments, the method results in a reduced brain amyloid level after administration relative to the brain amyloid level prior to the administration. In some embodiments, the brain amyloid level is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% relative to the brain amyloid level prior to said administration.

In some embodiments, the reduction of amyloid in the brain is determined by imaging using binding of radiotracers for brain Abeta amyloid and visualized with PET. In some embodiments, the reduction of amyloid in the brain is determined by imaging using binding of radiotracers for brain Abeta amyloid and visualized with PET. In some embodiments, the reduction in the adjusted mean change from baseline is at least 40 Centiloids (−40 Centiloids from baseline), such as at least 65 Centiloids (−65 Centiloids from baseline) or at least 90 Centiloids (−90 Centiloids from baseline) after 3 months of administration of the composition comprising a therapeutically effective amount of at least one bispecific anti-Abeta/TfR antibody. In some embodiments, the reduction in the adjusted mean change from the subject's level prior to the administration of the composition comprising a therapeutically effective amount of at least one bispecific anti-Abeta/TfR antibody is at least 50 Centiloids (−50 Centiloids from baseline), such as at least 55 Centiloids (−55 Centiloids from baseline) or at least 59 Centiloids (−59 Centiloids from baseline) after 12 months of administration of the composition comprising a therapeutically effective amount of at least one bispecific anti-Abeta/TfR antibody. In some embodiments, the reduction in the adjusted mean change from the subject's level prior to the administration of the composition comprising a therapeutically effective amount of at least one bispecific anti-Abeta/TfR antibody is at least 60 Centiloids (−60 Centiloids from baseline), such as at least 65 Centiloids (−65 Centiloids from baseline) or at least 70 Centiloids (−70 Centiloids from baseline) after 18 months of administration of the composition comprising a therapeutically effective amount of at least one bispecific anti-Abeta/TfR antibody.

In some embodiments, the method results in a reduced cerebrospinal fluid Abeta1-42 protein fragment level relative to the cerebrospinal fluid Abeta1-42 level protein fragment prior to the administration. In some embodiments, the method results in a reduction of cerebrospinal fluid Abeta1-42 protein fragment level of at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% relative to the cerebrospinal fluid Abeta1-42 protein fragment level prior to the administration.

In some embodiments, administration of the composition comprising at least one bispecific anti-Abeta/TfR antibody results in a brain amyloid level reduction of −0.20 to −0.60, such as from −0.20 to −0.40 as determined by visual reads of amyloid PET images, relative to the brain amyloid level prior to administration of the composition comprising at least one bispecific anti-Abeta/TfR antibody. In some embodiments, administration of the composition comprising at least one bispecific anti-Abeta/TfR antibody results in a brain amyloid level reduction of at least −0.25, as determined by visual reads of amyloid PET images, relative to the brain amyloid level prior to administration of the composition comprising at least one bispecific anti-Abeta/TfR antibody. In some embodiments, administration of the composition comprising at least one bispecific anti-Abeta/TfR antibody results in a brain amyloid level reduction of at least −0.30, as determined by visual reads of amyloid PET images, relative to the brain amyloid level prior to administration of the composition comprising at least one bispecific anti-Abeta/TfR antibody. In some embodiments, administration of the composition comprising at least one bispecific anti-Abeta/TfR antibody results in a brain amyloid level reduction of at least −0.60, as determined by visual reads of amyloid PET images, relative to the brain amyloid level prior to administration of the composition comprising at least one bispecific anti-Abeta/TfR antibody.

In some embodiments, administration of the composition comprising at least one bispecific anti-Abeta/TfR antibody results in at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% of the subjects being amyloid negative, as determined by visual reads of amyloid PET images.

In some embodiments, administration of the composition comprising at least one bispecific anti-Abeta/TfR antibody results in 75% to 100%, such as 80% to 100% or 85% to 100% of the subjects being amyloid negative, as determined by visual reads of amyloid PET images. In some embodiments, administration of the composition comprising at least one bispecific anti-Abeta/TfR antibody results in at least 75%, such as at least 80% or at least 85%, of the subjects being amyloid negative, as determined by visual reads of amyloid PET images. In some embodiments, administration of the composition comprising at least one bispecific anti-Abeta/TfR antibody results in 100% of the subjects being amyloid negative, as determined by visual reads of amyloid PET images.

In some embodiments, at least 75%, such as at least 80% or at least 85%, of the subjects are amyloid negative, as determined by visual reads of amyloid PET images, after 12 months of administration of the composition comprising a therapeutically effective amount of at least one bispecific anti-Abeta/TfR antibody. In some embodiments, at least 75%, such as at least 80%, at least 85%, at least 90%, or at least 95%, of the subjects are amyloid negative, as determined by visual reads of amyloid PET images, after 18 months of administration of the composition comprising a therapeutically effective amount of at least one bispecific anti-Abeta/TfR antibody. In some embodiments, the composition comprises 1.8 to 7.2 mg/kg, such as 1.8 mg/kg, 2.6 mg/kg, 3.6 mg/kg or 7.2 mg/kg, of at least one bispecific anti-Abeta/TfR antibody and is administered once every four weeks or once every month. In one preferred embodiment, the at least one bispecific anti-Abeta/TfR antibody is trontinemab.

### The Clinical Results

A key hurdle in the development of novel anti-Abeta monoclonal antibody (mAb) therapies is their delivery to the brain through the blood-brain-barrier (BBB). Due to the large size of mAbs, only a small percentage (−0.1%-0.2%) of peripherally administered antibodies reach the brain. Monoclonal antibodies appear to enter the brain passively, e.g., via the choroid plexus and distribute slowly to different brain regions.

Gantenerumab, an anti-amyloid monoclonal antibody, was evaluated in several clinical trials. In the SCarlet RoAD clinical trial (ClinicalTrials.gov identifier: NCT10224106; conversion from a gantenerumab phase 2/3 study in prodromal AD to an open-label extension and rollover into the Open RoAD open-label study; prodromal AD, CDR 0.5) gantenerumab was administered subcutaneously at a final dose of 1,200 mg once every four weeks (Q4W). In the Marguerite RoAD clinical trial (ClinicalTrials.gov identifier: NCT02051608; conversion from a gantenerumab phase 3 study in mild AD to an open-label extension and rollover into the Open RoAD open-label study; mild AD, CDR 1.0) gantenerumab was administered subcutaneously at a final dose of 1,200 mg once every four weeks (Q4W). In the GRADUATE I and II phase 3 studies (ClinicalTrials.gov identifiers: NCT03444870 and NCT03443973; two global, parallel, multicenter, randomized, double-blind, placebo-controlled studies of gantenerumab in patients with early AD (prodromal or MCI-AD to mild AD)) and the post-GRADUATE (ClinicalTrials.gov identifier: NCT04374253; open-label extension study) gantenerumab was administered subcutaneously at a final dose of 510 mg every second week (Q2W). In the GRADUATION phase 2 study (ClinicalTrials.gov identifier: NCT04592341; multicenter, open-label, single-arm study) gantenerumab was administered subcutaneously at a final dose of 255 mg once weekly (Q1W). In the SKYLINE phase 3 study (ClinicalTrials.gov identifier: NCT05256134; randomized, double-blind, placebo-controlled secondary prevention trial of gantenerumab in participants at risk or at the earliest stages of AD) gantenerumab was administered subcutaneously at a monthly dose of 1,020 mg with flexible administration (Q1W or Q2W). (See, e.g., Bateman, R. J., et al., Alzheimer's Res. Ther. 14 (2022) 178.) Brainshuttle™ (BrS) is a technology that has been developed to increase penetration of large molecules such as antibodies into the brain. Access of large molecules to the brain is restricted by the blood-brain-barrier (BBB), a gatekeeper between the blood and the brain tissue that carefully filters which molecules can enter the brain. By using a specific antibody format antibodies have been created that are able to cross the blood-brain-barrier by binding to one of the protein receptors located on its surface, i.e. the human transferrin receptor 1 (TfR1, CD71). The so-called “Brainshuttle™” technology allows the transport of all types of therapeutic molecules into the brain, regardless of their intrinsic ability to cross the blood-brain-barrier.

Trontinemab (also known as RG6102 or RO 7126209; see also proposed INN List 127, Cas 2568868-35-7, USAN KL-203, FDA UNII N0GBZ1GWC2) is a BrS antibody employing the specific “2+1” design. In more detail, it is bispecific antibody (see WO 2017/055540, expressly incorporated by reference herein) consisting of a variant of the anti-amyloid monoclonal antibody gantenerumab, a bivalent, monospecific, y-shaped fully human monoclonal antibody targeting the amyloid-beta protein and an additional domain-exchanged Fab specifically binding to human transferrin receptor 1 (anti-TfR 1 Fab), that is conjugated to the C-terminus of one of the heavy chains of the gantenerumab variant.

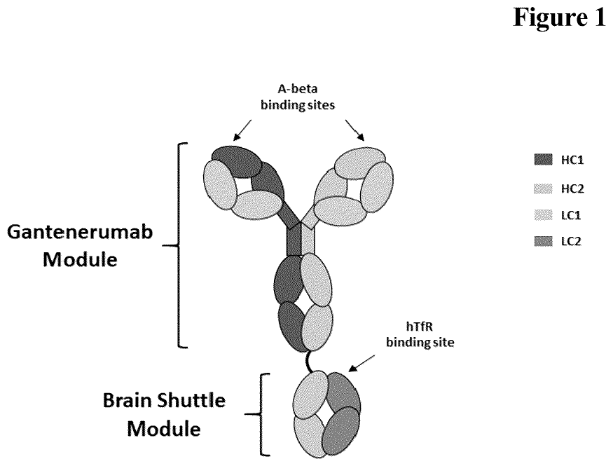

The structure of trontinemab is shown in FIG. 1.

Trontinemab has been developed for the treatment of Alzheimer's disease. It is administered intravenously and, while circulating in the bloodstream, it binds to the transferrin receptor present on endothelial cells that make up the blood-brain-barrier. This leads to its active endocytosis, transport across the blood-brain-barrier and release into the brain parenchyma.

Preclinical work has been done using a “mouse version” of trontinemab, wherein a gantenerumab variant has been fused to a single Fab that binds to the mouse transferrin receptor. In an AD mouse models, a substantially increase in brain concentration could be observed (Niewoehner, J., et al., Neuron 8 (2014) 49-60). Up to 40-times more BrS-antibody entered the brain and bound to amyloid plaques, compared with a standard y-shaped IgG antibody. Trontinemab stimulated plaque clearance by immune cells at lower doses compared to the parent antibody gantenerumab. At the same time, no immune responses to endothelial cells or other transferrin receptor-expressing cells were initiated.

Surrogate trontinemab (BrS-mAb31), which cross-react with mouse TfR1, and its surrogate counterpart molecule (mAb31) were used in mouse in vivo evaluation of trontinemab. The BrS-mAb31 surrogate binds to a similar epitope as trontinemab, at the apical domain on TfR1 outside the binding interface between TfR1 and transferrin (Tf). The mouse surrogate of the BrS construct was comprised of a single Fab of a rat antibody specific for mouse TfR1 fused to the C-terminal Fc-region end of mAb31, a human IgG1 antibody specific for Abeta fibrils and oligomers (the preclinical version of gantenerumab). This BrS-mAb31, which uses a monovalent binding mode to the TfR1, was shown to boost plaque decoration in a mouse model of AD compared to the parent antibody (Niewoehner et al. 2014, see above).

In a transgenic amyloidosis mouse model, BrS-mAb31 showed lower plasma exposure than the non-BrS analog mAb31; this was attributed to target-mediated drug disposition through TfR binding in the periphery. In a 4-month treatment study using weekly dosing, BrS-mAb31 showed about 12-fold higher brain exposure than mAb31 at 7 days after the last injection. In addition, BrS-mAb31 resulted in significantly greater reductions of Abeta protein amyloid plaques in cortex and hippocampus compared with vehicle controls and equimolar low-dose mAb31, even though plasma exposure for the BrS construct was thus lower. In in vitro studies, it was demonstrated that effector function of BrS-mAb31 was hidden in the periphery due to an inverted TfR-binding mode, primarily because of steric hindrance, thereby reducing potential for first infusion reactions.

It has to be pointed out that there is no established animal model for AD in non-human primate (NHP) available.

With trontinemab a substantially higher brain exposure due to shuttling could be achieved (Kulic, L., et al. Presented at AD/PD 2021, virtual conference).

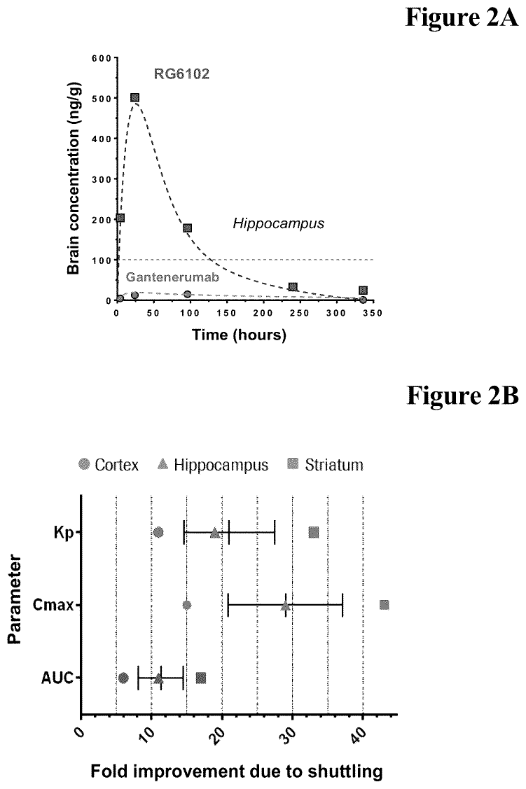

A 6- to 42-fold higher brain (tissue) exposure in non-human primates, especially in deep brain regions, compared to gantenerumab could be achieved with a single IV dose of 10 mg/kg trontinemab versus a single IV dose of 20 mg/kg gantenerumab. This is shown in FIG. 2A and the following table.

| approx. | | | |
|---|---|---|---|
| improvement | Cortex | Hippocampus | Striatum |
| Kp | 11-fold | 18-fold | 33-fold |
| Cₘₐₓ | 15-fold | 38-fold | 43-fold |
| AUC | 6-fold | 11-fold | 17-fold |

AUC, area under the curve;
Cₘₐₓ, maximum observed concentration;
KP, partition coefficient.

The brain to plasma ratio in different regions of the brain ranged from 0.46% to 0.78%, which corresponds to a 6- to 17-fold higher brain area under the concentration-time curve (AUC) at steady state than that of gantenerumab (see FIG. 2B).

However, it remained questionable whether it will be possible by using Brainshuttle™ technology to achieve a more extensive and homogenous brain penetration, with faster amyloid plaque clearance, in humans compared to antibodies not using an active TfR1 receptor-mediated transcytosis through the blood-brain-barrier.

In the first in-human study of trontinemab (Phase Ia, single ascending dose (SAD)) in healthy human volunteers doses of 0.1 mg/kg to 7.2 mg/kg were tested (0.1 mg/kg and 0.4 mg/kg: four active and two placebo each; 1.2 mg/kg, 3.6 mg/kg and 7.2 mg/kg: six active and two placebo each). The results thereof are outlined in the following.

A dose proportional increase in plasma pharmacokinetics (PK) with trontinemab was observed. Additionally, an eightfold increase in CSF/plasma ratio compared with conventional IgG mAbs was observed (0.5-1.2% vs. 0.1-0.2%). Trontinemab doses from 0.1-3.6 mg/kg were generally well tolerated. All observed adverse events (AEs) were classified as either Grade 1 or Grade 2 in intensity and were resolved. No Grade 3, 4 or 5 AEs were observed. The most frequent AEs considered related to study treatment were infusion-related reactions, headache, and nausea. No serious AEs (including with fatal outcome) were reported up to the maximally tested dose level of 7.2 mg/kg.

The second in-human study of trontinemab (Phase Ib/IIa, multiple ascending dose (MAD); NCT04639050, BP42155, EudraCT 2020-002477-98) was performed in participants with prodromal or mild-to-moderate Alzheimer's Disease (AD). It was a randomized, global multicenter, double-blind, placebo-controlled, parallel-group study with a staggered, parallel-group design, with 4 initial sequential Cohorts.

Doses of 0.2 mg/kg, 0.6 mg/kg, 1.8 mg/kg and 3.6 mg/kg were tested (each dose Cohort with at least eight active participants and at least two participants on placebo, up to a maximum of 120 participants).

The study consists of a continuous screening period (up to 12 weeks, including a one week baseline period), a double-blind treatment period (28 weeks), and a safety follow-up period (28 weeks). It used a staggered parallel-group design, with participants recruited in four sequential dose Cohorts (selected dose levels: 0.2 mg/kg (Cohort 1), 0.6 mg/kg (Cohort 2), 1.8 mg/kg (Cohort 3), and 3.6 mg/kg (Cohort 4)).

Trontinemab was applied intravenously, once every 4 weeks for 28 weeks (primary endpoint; total of seven doses) with a safety follow-up period of 28 weeks.

The participants fulfilled the following criteria:
age: 50 years to 85 years (adult, older adult);
mild to moderate AD dementia (consistent with National Institute on Aging-Alzheimer's Association [NIA-AA] core clinical criteria for probable AD dementia) or prodromal AD (consistent with the NIA-AA diagnostic criteria and guidelines for mild cognitive impairment due to AD);
Mini-Mental State Examination (MMSE) score of 18 to 28 points, inclusive, within 84 days before baseline;
Clinical Dementia Rating-Global Score (CDR-GS) of 0.5, 1, or 2 within 84 days before baseline;
positive amyloid PET scan (cut-off: >50 Centiloid units) within 12 months before baseline.

Sentinel dosing was applied to each Cohort. Amyloid plaque burden was assessed by florbetapir and florbetaben PET imaging at screening, day 78 (only Cohort 3 and Cohort 4), and day 196. An interim analysis of the amyloid PET results was performed prior to escalating to the maximum dose level of 3.6 mg/kg (Cohort 4).

In the participants, the brain amyloid load was determined by amyloid positron emission tomography (PET) at baseline (week 0) and dosing week 12 (Cohorts 3+4) and dosing week 28 (Cohorts 1-4). Also the concentration of trontinemab in plasma (up to 32 weeks) and cerebral spinal fluid (CSF) (baseline, dosing week 25) was determined.

Additionally, CDR and MMSE (baseline, weeks 28 and 40) were determined to evaluate clinical efficacy.

Change from baseline in PET and ASL-MRI (arterial spin labeling magnetic resonance imaging) were determined for evaluating cerebral perfusion. In blood and CSF change from baseline Abeta1-42 protein fragment (amyloid beta peptide 1-42; SEQ ID NO: 45), tTau (total tau protein), pTau (phosphorylated tau protein), neurogranin, NfL (neurofilament light protein), and sTREM2 (soluble triggering receptor expressed on myeloid cells 2) amongst others were determined as biomarkers (blood: baseline, weeks 11, 24, 25, and 26; CSF: baseline, week 25).

The following was found:
The interim analysis for safety and amyloid PET was based on a data snapshot as of Jan. 18, 2023. The safety results included blinded data from a total of 44 participants who received trontinemab or placebo. The vast majority of adverse events (AEs) were mild (grade 1) or moderate (grade 2) in severity. Consistent with previous results from the Phase 1a single ascending dose study of trontinemab, infusion related reactions (IRRs) were the most common treatment-emergent AEs. All IRRs were mild or moderate in severity.
The interim amyloid PET results included data from 36 participants (11 men, 25 women) enrolled in Cohorts 1 to 3, who underwent at least one post-baseline amyloid PET scan. A significant dose-dependent amyloid plaque lowering was observed across all three analyzed dose levels. In Cohort 3 (1.8 mg/kg) a strong amyloid plaque reduction and amyloid PET negativity in several participants (cut off: <24 Centiloids) were documented already at day 78.
Trontinemab administered with a dose of 1.8 mg/kg led to three times faster and more extensive amyloid lowering than other standard monoclonal antibodies with good safety and low ARIA risk. This is expected to translate into superior clinical efficacy.
Reaching the amyloid negative threshold (or going below the amyloid negative threshold value) is understood to translate into a clinical effect; for example a 70% amyloid negativity rate after 28 weeks of treatment with a dosing every four weeks is understood to translate into a 44% CDR-SB RR versus placebo at 18 months (Clinical Dementia Rating Sum of Boxes Relative Reduction), whereas a 90% amyloid negativity rate after 28 weeks of treatment with an every four weeks dosing is understood to translate into 50% or more CDR-SB RR versus placebo at 18 months (Scelsi et al, CTAD 2023).

| % below | Model predicted |
|---|---|
| threshold | CDR-SB RR |
| 50 | 33.4% |
| 60 | 38.5% |
| 70 | 43.6% |
| 80 | 48.8% |
| 90 | 53.9% |

The participants of the different study Cohorts had the following characteristics:

| | Cohort 1 | | Cohort 2 | | Cohort 3 | | Cohort 4 | |
|---|---|---|---|---|---|---|---|---|
| baseline | 0.2 mg/ | | 0.6 mg/ | | 1.8 mg/ | | 3.6 mg/ | |
| demographic | kg or | | kg or | | kg or | | kg or | |
| and disease | placebo | | placebo | | placebo | | placebo | |
| characteristics | (n = 14) | | (n = 14) | | (n = 16) | | (n = 15) | |
| age, mean | 70.0 | (7.4) | 68.6 | (9.2) | 72.4 | (8.0) | 71.9 | (5.3) |
| (SD) [years] | | | | | | | | |
| sex, female, n | 12 | (85.7%) | 7 | (50.0%) | 10 | (62.5%) | 9 | (60%) |
| [%] | | | | | | | | |
| white skin | 14 | (100%) | 14 | (100%) | 16 | (100%) | 14 | (93.3%) |
| color, n [%] | | | | | | | | |
| CDR-GS, n | | | | | | | | |
| [%] | | | | | | | | |
| 0.5 | 4 | (28.6%) | 6 | (42.9%) | 8 | (50.0%) | 7 | (50.0%) |
| 1 | 6 | (42.9%) | 8 | (57.1%) | 7 | (43.8%) | 7 | (50.0%) |
| 2 | 4 | (28.6%) | 0 | | 1 | (6.2%) | 0 | |
| CDR-SB, | 5.8 | (2.8) | 4.8 | (1.9) | 5.3 | (2.9) | 4.8 | (1.4) |
| mean (SD) | | | | | | | | |
| MMSE, mean | 20.9 | (3.2) | 20.4 | (4.7) | 19.8 | (2.8) | 20.7 | (2.4) |
| (SD) | | | | | | | | |
| ApoE ε4 | | | | | | | | |
| number of | | | | | | | | |
| allels, n [%] | | | | | | | | |
| 0 | 4 | (28.6%) | 7 | (50.0%) | 6 | (37.5%) | 5 | (33.3%) |
| 1 | 7 | (50.0%) | 6 | (42.9%) | 8 | (50.0%) | 7 | (46.7%) |

| | | | | | | | |
|---|---|---|---|---|---|---|---|
| 2 | 3 | (21.4%) | 0 | 2 | (12.5%) | 3 | (30.0%) |

| | | | | | |
|---|---|---|---|---|---|
| missing data | 0 | 1 | (7.1%) | 0 | 0 |

The multiple-dose pharmacodynamics of trontinemab showing mean change from baseline in amyloid PET Centiloids by nominal visit are as follows. For participants on active treatment, the mean change from baseline determined 28 days after administration of Dose 7 was −20.2 Centiloids (reduction by 20.2 Centiloids from baseline; range: −49.4 to −0.7) in the 0.2 mg/kg Cohort and −27.6 Centiloids (reduction by 27.6 Centiloids from baseline; range: −52.3 to −1.4) in the 0.6 mg/kg Cohort (mean number of doses received: 6.6 and 6.2, respectively). In the Cohort receiving 1.8 mg/kg, a more pronounced mean change from baseline was seen based on the data at the time of the data snapshot. The mean change from baseline determined 22 days after administration of nominal Dose 3 was −65.3 Centiloids (reduction by 65.3 Centiloids from baseline; range: −102.8 to −42.6) in the 1.8 mg/kg Cohort (after a mean number of doses received: of 2.8).

| dose Q4W | number | mean number | mean change | 90% |
|---|---|---|---|---|
| 28 days after | of | of received | from baseline | confidence |
| Dose 7 | participants | doses | in Centiloids | interval |
| 0 mg/kg | 7 | 7 | −1 | +/−15 |
| 0.2 mg/kg | 10 | 6.6 | −20 | +/−7 |
| 0.6 mg/kg | 9 | 6.2 | −28 | +/−9 |
| dose Q4W | number | mean number | mean change | 90% |
| 22 days after | of | of received | from baseline | confidence |
| Dose 3 | participants | doses | in Centiloids | interval |
| 0 mg/kg | 3 | 3 | +9 | +/−22 |
| 1.8 mg/kg | 9 | 2.8 | −65 | +/−13 |

Thus, it has surprisingly been found that rapid amyloid plaque clearance can be achieved at significantly lower dose levels than with typical anti-amyloid monoclonal antibodies.

These results show that trontinemab rapidly lowers amyloid plaques burden in a dose-dependent fashion. In a majority of patients the amyloid plaque load was reduced to a level comparable to people not suffering from AD and in short time.

In more detail, it was found that a monthly intravenous dose of 1.8 mg/kg led to a rapid and substantial amyloid lowering of 84 Centiloids (−84 Centiloids from baseline) after 7 months.

This is faster than with standard anti-Abeta monoclonal antibodies; 72% of the participants are below the Abeta protein positivity threshold (24.1 CL) at week 28.

In more detail, it was found that a monthly intravenous dose of 3.6 mg/kg led to a rapid and substantial amyloid lowering of 91 Centiloids (−91 CL from baseline) after 3 months (n=8).

This is ˜6 times faster than with standard anti-Abeta monoclonal antibodies; 6/8 (63%) participants are below the Abeta protein positivity threshold (24.1 CL) at week 12.

The materially improved safety and tolerability profile of trontinemab, relative to standard anti-Abeta monoclonal antibodies, could be confirmed with this data. Infusion-related reactions (IRRs) remain the most prominent side effect (7/15; 46.7%) with common symptoms of chills, fever and headache and can be mitigated by appropriate premedication, in one embodiment with a pre-medication with corticosteroids or pre-medication with acetaminophen/NSAID and dexamethasone. NSAID=non-steroidal anti-inflammatory drug.

None of the Cohort 4 participants showed ARIA-E so far (the overall ARIA-E rate in the study is 2/59—both cases were radiographically mild and reversible). A transient mild anemia was observed in 5 participants in Cohort 3 and in one participant in Cohort 4. Trends of decreasing mean hemoglobin levels and decreasing red blood cell counts were recorded in all treatment groups (including placebo), suggesting that frequent blood collection likely significantly contributed to the anemia phenotype.

Thus, in one embodiment, trontinemab has a total ARIA-E rate of less than 13% of treated subjects and a symptomatic ARIA-E rate of 3% or less of treated subjects. This low ARIA-E rate translates into a short ARIA-E risk period due to faster amyloid removal.

In certain embodiment, trontinemab is administered without titration. Without being bound by this theory, it is assumed that this rapid amyloid plaque clearance with trontinemab can be achieved by using the TfR1-based Brainshuttle™ approach.

It was found that 4 of 11 participants (36%) were below the amyloid positivity threshold of 24.1 Centiloids at week 11. At week 28 already 6 of 8 participants (75%) were below the amyloid positivity threshold of 24.1 Centiloids and 5 of 8 participants (63%) were below 11 Centiloids.

In contrast thereto, participants receiving doses of 0.2 mg/kg and 0.6 mg/kg had mean amyloid values in Centiloids at week 28 of 74 and 56, respectively, with none of the participants in the 0.2 mg/kg dosing group and only one of the participants in the 0.6 mg/kg dosing group being below the amyloid positivity threshold of 24.1 Centiloids.

| | Mean amyloid value in CL | | | | | | | | |
|---|---|---|---|---|---|---|---|---|---|
| | (% amyloid negative (<24.1 CL)) | | | | | | | | |
| | | | 0.2 | 0.6 | | 1.8 | | 3.6 | |
| | Placebo | | mg/kg | mg/kg | | mg/kg | | mg/kg | |
| Baseline | 95-98 | (0%) | 91 (0%) | 88 | (0%) | 100 | (0%) | 119 | (0%) |

| Week 11 | 128 | (0) | — | — | 40 | (36) | — | |
|---|---|---|---|---|---|---|---|---|
| Week 12 | 119 | (0%) | — | — | 40 | (36%) | 21 | (63%) ** |

| | | | | | | | | |
|---|---|---|---|---|---|---|---|---|
| Week 28 | 91 | (0%) | 74 (0%) | 56 | (10%) | 9 | (75%)* | — |

* 6/8 (75% of participants) <24.1 CL, 5/8 (63%) <11 CL at 1.8 mg/kg after 28 weeks
** 5/8 (63% of participants) <24.1 CL, 4/8 (50%) <11 CL at 3.6 mg/kg after 12 weeks

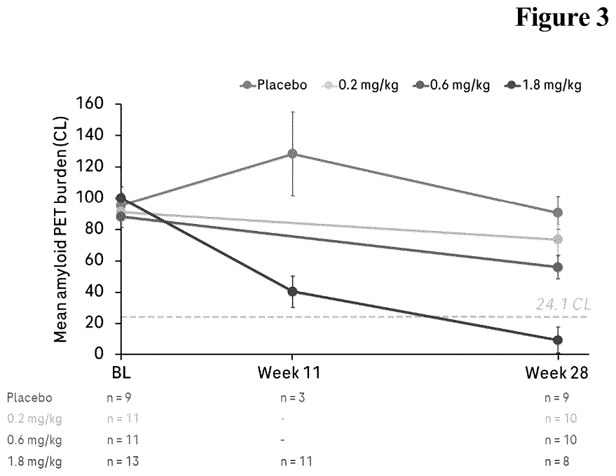

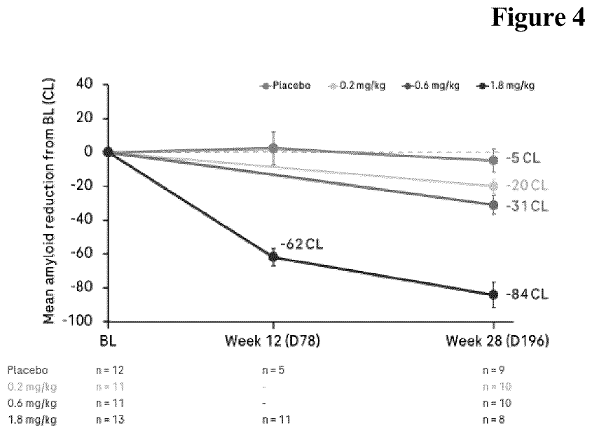

The results for Cohorts 1 to 3 are visualized in FIGS. 3 (mean amyloid PET burden in Centiloids) and 4 (mean amyloid reduction from baseline in Centiloids. The study was a randomized (4:1) with 10 or more participants per group (active:placebo). All participants were of white skin and had a body weight between 60 and 70 kg. The APOE ε4 carrier status was 71% for the dosing of 0.2 mg/kg, 43% for the dosing of 0.6 mg/kg and 63% for the dosing of 1.8 mg/kg.

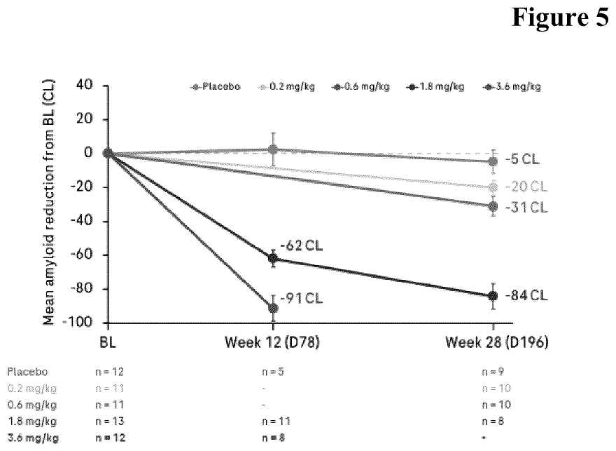

The results, including the results for Cohort 4 after 12 weeks, are visualized in FIG. 5 (mean amyloid reduction from baseline in Centiloids). The study was a randomized (4:1) with 10 or more participants per group (active:placebo). All participants were of white skin except for one in Cohort 4 and had a body weight between 68 and 72.5 kg. The APOE ε4 carrier status was about 71% for the dosing of 0.2 mg/kg, about 43% for the dosing of 0.6 mg/kg, about 63% for the dosing of 1.8 mg/kg and about 67% for the dosing of 3.6 mg/kg.

It can be seen that with doses of 1.8 mg/kg and 3.6 mg/kg a reduction by at least 60 Centiloids to more than 90 Centiloids after 12 weeks and by at least 80 Centiloids to more than 90 Centiloids after 28 weeks could be achieved.

In the following table the results obtained with trontinemab are shown together with those of other anti-Abeta antibodies. It has to be pointed out that amyloid PET burden is in Centiloids, which is variable across studies. However, lower baseline levels in CLARITY-AD (lecanemab) enabled faster crossing of the amyloid positivity threshold, even though the absolute change was comparable to the GRADUATE studies.

| | | | baseline | change | |
|---|---|---|---|---|---|
| | | | amyloid | amyloid | |
| | dosing | | PET | PET | |
| antibody | schedule | dose | (Centiloids) | (Centiloids) | at |
| trontinemab | Q4W | 1.8 mg/kg (IV) | 96 | −65 | day 78 |
| gantenerumab | Q2W | 510 mg (SC) | 91.8 | −57.6 | week 116 |
| (GRADUATE I) | | (=7.3 mg/kg | | | |
| | | @ 70 kg bw) | | | |
| gantenerumab | Q2W | 510 mg (SC) | 92.9 | −46.8 | week 116 |
| (GRADUATE II) | | (=7.3 mg/kg | | | |
| | | @ 70 kg bw) | | | |
| aducanumab | Q4W | 10 mg/kg | 90.8 | −53.5 | week 78 |
| (ENGAGE) | | | | | |
| lecanemab | Q2W | 10 mg/kg | 78 | −55.5 | week 78 |
| (CLARITY AD) | | | | | |
| donanemab | Q4W | 20 mg/kg | 108 | −70 | week 24 |
| (TRAIL- | | (IV) | | −84 | week 76 |
| BLAZER-ALZ) | | | | | |
| aducanumab | Q4W | 10 mg/kg | 85.3 | −64.2 | week 78 |
| (EMERGE) | | | | | |

bw = body weight

It has to be expressly pointed out that one third of the patients were PET negative already after 12 weeks (every four weeks dosing; Q4W) of administration of trontinemab at a dose of 1.8 mg/kg or 3.6 mg/kg.

The following events occurred during the study until Oct. 23, 2023.

| | Cohort 1 | Cohort 2 | Cohort 3 | Cohort 4 |
|---|---|---|---|---|
| Total number of | 0.2 mg/kg | 0.6 mg/kg | 1.8 mg/kg | 3.6 mg/kg |
| participants with at | or placebo | or placebo | or placebo | or placebo |
| least one event, (%) | (n = 14) | (n = 14) | (n = 16) | (n = 15) |
| infusion related | 1 (1.7%) | 4 (28.6%) | 12 (75.0%) | 7 (46.7%) |
| reaction (IRR) * | | | | |
| Anemia ** | 2 (14.3%) | 0 | 5 (31.2%) | 1 (6.7%) |

* Common IRR symptoms include fever, chills, and headache. In Cohorts 1-3, most IRRs occurred after administration of the first study drug dose (without premedication), were mild to moderate in severity and resolved with or without appropriate medication. Subsequently, routine premedication with paracetamol/nonsteroidal anti-inflammatory drugs was implemented in Cohorts 3 and 4, which reduced the incidence and symptoms of IRRs.
** A transient mild anemia was observed in 5 participants in Cohort 3 and in one participant in Cohort 4. Trends of decreasing mean hemoglobin levels and decreasing red blood cell counts were recorded in all treatments groups (including placebo), suggesting that frequent blood collection likely significantly contributed to the anemia phenotype.

Only two cases of ARIA occurred so far in the Cohort with 1.8 mg/kg dosing in the 12 participants (3 placebo, 9 dosed) (1 amyloid related imaging abnormalities-edema (ARIA-E), mild, resolved within 1 week without medication; 1 amyloid related imaging abnormalities-hemosiderosis (ARIA-H), asymptomatic).

Abeta protein plaque decoration correlates with cumulative doses and, thus, TfR1 transport across the BBB is not desensitized by multiple dosing.

The ARIA event data is shown in the following table with the number of participants with events listed.

| Total number of | Cohort 1 | Cohort 2 | Cohort 3 | Cohort 4 |
|---|---|---|---|---|
| participants with | 0.2 mg/kg or | 0.6 mg/kg or | 1.8 mg/kg or | 3.6 mg/kg or |
| event [events | placebo | placebo | placebo | placebo |
| per participant], (%) | (n = 14) | (n = 14) | (n = 16) | (n = 15) |
| ARIA-E * | 0 | 0 | 1 (two times) | 0 |
| | | | (6.7%) | |
| ARIA-H ** | 0 | 0 | 0 | 0 |
| microhemorrhage | | | | |
| leptomening-eal | 0 | 0 | 1 (two times) | 0 |
| hemosider-osis | | | (6.7%) | |
| (LH) | | | | |
| ARIA-E with | 0 | 0 | 0 | 0 |
| concurrent ARIA-H | | | | |
| Macrohemorrhage | 0 | 0 | 0 | 0 |

* One participant in Cohort 3 developed two episodes of ARIA-E: first, on routine Day 22 MRI scan, radiographically mild, temporally associated with mildly impaired attention over approximately one week, complete radiographic resolution within 4 weeks; second, on routine on Day 281 MRI, radiographically mild+, asymptomatic, complete radiographic resolution within 8 weeks.
** One participant in Cohort 3 developed 2 asymptomatic ARIA-H findings not concurrent with ARIA-E; one left occipital LH (12 mm) on routine Day 162 MRI, then one right frontal LH (8 mm) on routine Day 281 MRI.

Trontinemab is a novel Brainshuttle™ bispecific anti-TfR/Aβ antibody that crosses the blood brain barrier via active TfR1 mediated transcytosis at the capillary level. In people with AD, trontinemab demonstrated rapid and robust amyloid plaque reduction at doses lower than known for traditional non-Brainshuttle™ standard IgG antibodies and surprisingly lower than expected (i.e., already at 1.8 mg/kg).

Preliminary results show a substantial reduction already at a dose of 1.8 mg/kg with further acceleration of amyloid plaque reduction at 3.6 mg/kg and amyloid negativity in a majority of participants already after 12 weeks of treatment.

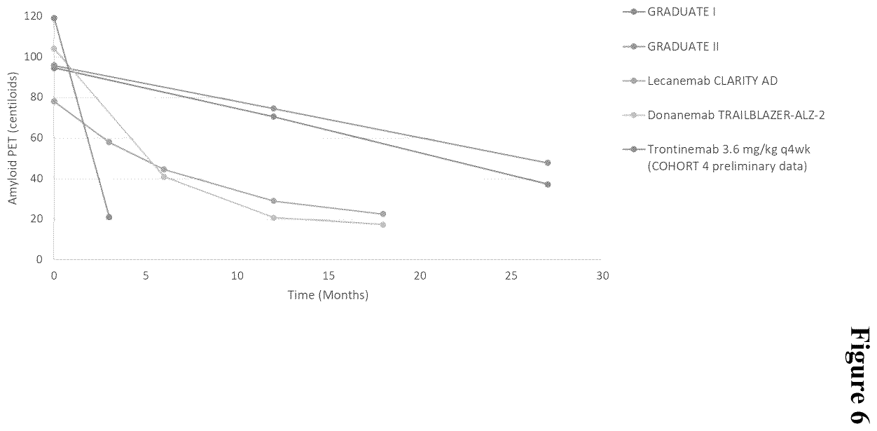

Sustained low ARIA incidence (no ARIA-E/ARIA-H at 3.6 mg/kg so far), which are lower than known from the art for anti-Abeta antibodies and overall favorable safety and tolerability profile show the improved properties of trontinemab (FIG. 6).

Thus, trontinemab provides for an anti-amyloid immunotherapy by inducing a high clearance of amyloid load, as measured with positron emission tomography, in the brain of early-stage biomarker-proven AD patients.

It has been established that the rate of amyloid clearance (as measured with amyloid PET) observed in patients under anti-amyloid therapies is linked to a significant slowing of cognitive decline on several cognitive scales, eventually in a dose-dependent manner.

Thus, trontinemab can be/is a disease-modifying drug that delays neurodegeneration and neuronal cell death by acting on the pathological mechanism of Alzheimer's Disease and, as a result, inhibit the progression of clinical symptoms, i.e. showing a relationship between changes in clinical symptoms and changes in biomarkers. Thus, trontinemab can be/is helpful in slowing progression of mild cognitive impairment and mild dementia due to Alzheimer's disease with an improved safety profile. Therefore, the indication of trontinemab can be/is to slow the progression of mild cognitive impairment and mild dementia due to Alzheimer's disease.

In addition to the various embodiments depicted and claimed, the disclosed subject matter is also directed to other embodiments having other combinations of the features disclosed and claimed herein. As such, the particular features presented herein can be combined with each other in other manners within the scope of the disclosed subject matter such that the disclosed subject matter includes any suitable combination of the features disclosed herein. The foregoing description of specific embodiments of the disclosed subject matter has been presented for purposes of illustration and description. It is not intended to be exhaustive or to limit the disclosed subject matter to those embodiments disclosed.

Unless otherwise defined herein, scientific and technical terms used in connection with the current invention shall have the meanings that are commonly understood by those of ordinary skill in the art. Further, unless otherwise required by context, singular terms shall include pluralities and plural terms shall include the singular.

As used herein, the amino acid positions of all constant regions and domains of the heavy and light chain are numbered according to the Kabat numbering system described in Kabat, et al., Sequences of Proteins of Immunological Interest, 5th ed., Public Health Service, National Institutes of Health, Bethesda, MD (1991) and is referred to as “numbering according to Kabat” herein. Specifically, the Kabat numbering system (see pages 647-660) of Kabat, et al., Sequences of Proteins of Immunological Interest, 5th ed., Public Health Service, National Institutes of Health, Bethesda, MD (1991) is used for the light chain constant domain CL of kappa and lambda isotype, and the Kabat EU index numbering system (see pages 661-723) is used for the constant heavy chain domains (CH1, Hinge, CH2 and CH3, which is herein further clarified by referring to “numbering according to Kabat EU index” in this case).

The knobs into holes dimerization modules and their use in antibody engineering are described in Carter P.; Ridgway J. B. B.; Presta L. G.: Immunotechnology, Volume 2, Number 1, February 1996, pp. 73-73(1).

General information regarding the nucleotide sequences of human immunoglobulins light and heavy chains is given in: Kabat, E. A., et al., Sequences of Proteins of Immunological Interest, 5th ed., Public Health Service, National Institutes of Health, Bethesda, MD (1991).

Useful methods and techniques for carrying out the current invention are described in e.g. Ausubel, F. M. (ed.), Current Protocols in Molecular Biology, Volumes I to III (1997); Glover, N. D., and Hames, B. D., ed., DNA Cloning: A Practical Approach, Volumes I and II (1985), Oxford University Press; Freshney, R. I. (ed.), Animal Cell Culture—a practical approach, IRL Press Limited (1986); Watson, J. D., et al., Recombinant DNA, Second Edition, CHSL Press (1992); Winnacker, E. L., From Genes to Clones; N.Y., VCH Publishers (1987); Celis, J., ed., Cell Biology, Second Edition, Academic Press (1998); Freshney, R. I., Culture of Animal Cells: A Manual of Basic Technique, second edition, Alan R. Liss, Inc., N.Y. (1987).

The use of recombinant DNA technology enables the generation of derivatives of a nucleic acid. Such derivatives can, for example, be modified in individual or several nucleotide positions by substitution, alteration, exchange, deletion, or insertion. The modification or derivatization can, for example, be carried out by means of site directed mutagenesis. Such modifications can easily be carried out by a person skilled in the art (see e.g., Sambrook, J., et al., Molecular Cloning: A laboratory manual (1999) Cold Spring Harbor Laboratory Press, New York, USA; Hames, B. D., and Higgins, S. G., Nucleic acid hybridization—a practical approach (1985) IRL Press, Oxford, England).

All documents cited herein are expressly incorporated by reference herein.

### EXAMPLES

### Example 1: Phase Ta Study

In the first in-human study of trontinemab (Phase Ia, single ascending dose (SAD)) in healthy human volunteers doses of 0.1 mg/kg to 7.2 mg/kg were tested (0.1 mg/kg and 0.4 mg/kg: four active and two placebo each; 1.2 mg/kg, 3.6 mg/kg and 7.2 mg/kg: six active and two placebo each). The results thereof are outlined in the following.

A dose proportional increase in plasma pharmacokinetics (PK) with trontinemab was observed. Additionally, an eightfold increase in CSF/plasma ratio compared with conventional IgG mAbs was observed (0.5-1.2% vs. 0.1-0.2%). Trontinemab doses from 0.1-3.6 mg/kg were generally well tolerated. All observed adverse events (AEs) were classified as either Grade 1 or Grade 2 in intensity and were resolved. No Grade 3, 4 or 5 AEs were observed. The most frequent AEs considered related to study treatment were infusion-related reactions, headache, and nausea. No serious AEs (including with fatal outcome) were reported up to the maximally tested dose level of 7.2 mg/kg.

### Example 2: Phase Ib/IIa Study

The second in-human study of trontinemab (Phase Ib/IIa, multiple ascending dose (MAD); NCT04639050, BP42155, EudraCT 2020-002477-98) was in participants with prodromal or mild-to-moderate Alzheimer's Disease (AD). It was a randomized, global multicenter, double-blind, placebo-controlled, parallel-group study with a staggered, parallel-group design, with 4 initial sequential Cohorts.

Doses of 0.2 mg/kg, 0.6 mg/kg, 1.8 mg/kg and 3.6 mg/kg were tested (each dose Cohort with at least eight active participants and at least two participants on placebo, up to a maximum of 120 participants).

The study consisted of a continuous screening period (up to 12 weeks, including a one week baseline period), a double-blind treatment period (28 weeks), and a safety follow-up period (28 weeks). It used a staggered parallel-group design, with participants recruited in four sequential dose Cohorts (selected dose levels: 0.2 mg/kg (Cohort 1), 0.6 mg/kg (Cohort 2), 1.8 mg/kg (Cohort 3), and 3.6 mg/kg (Cohort 4)).

Trontinemab was applied intravenously, once every 4 weeks for 28 weeks (primary endpoint; total of seven doses) with a safety follow-up period of 28 weeks.

The participants fulfilled the following criteria:
age: 50 years to 85 years (adult, older adult);
mild to moderate AD dementia (consistent with National Institute on Aging-Alzheimer's Association [NIA-AA] core clinical criteria for probable AD dementia) or prodromal AD (consistent with the NIA-AA diagnostic criteria and guidelines for mild cognitive impairment due to AD);
Mini-Mental State Examination (MMSE) score of 18 to 28 points, inclusive, within 84 days before baseline;
Clinical Dementia Rating-Global Score (CDR-GS) of 0.5, 1, or 2 within 84 days before baseline;
positive amyloid PET scan (cut-off: >50 Centiloid units) within 12 months before baseline.

Sentinel dosing was applied to each Cohort. Amyloid plaque burden was assessed by florbetapir and florbetaben PET imaging at screening, day 78 (only Cohort 3 and Cohort 4), and day 196. An interim analysis of the amyloid PET results was performed prior to escalating to the maximum dose level of 3.6 mg/kg (Cohort 4).

In the participants, the brain amyloid load was determined by amyloid positron emission tomography (PET) at baseline (week 0) and dosing week 12 (Cohorts 3+4) and dosing week 28 (Cohorts 1-4). Also the concentration of trontinemab in plasma (up to 32 weeks) and cerebral spinal fluid (CSF) (baseline, dosing week 25) was determined.

Additionally, CDR and MMSE (baseline, weeks 28 and 40) were determined to evaluate clinical efficacy.

Change from baseline in PET and ASL-MRI (arterial spin labeling magnetic resonance imaging) were determined for evaluating cerebral perfusion. In blood and CSF change from baseline Abeta1-42 protein fragment (amyloid beta peptide 1-42; SEQ ID NO: 45), tTau (total tau protein), pTau (phosphorylated tau protein), neurogranin, NfL (neurofilament light protein), and sTREM2 (soluble triggering receptor expressed on myeloid cells 2) amongst others were determined as biomarkers (blood: baseline, weeks 11, 24, 25, and 26; CSF: baseline, week 25).

The following was found:
The interim analysis for safety and amyloid PET was based on a data snapshot as of Jan. 18, 2023. The safety results included blinded data from a total of 44 participants who received trontinemab or placebo. The vast majority of adverse events (AEs) were mild (grade 1) or moderate (grade 2) in severity. Consistent with previous results from the Phase 1a single ascending dose study of trontinemab, infusion related reactions (IRRs) were the most common treatment-emergent AEs. All IRRs were mild or moderate in severity.
The interim amyloid PET results included data from 36 participants (11 men, 25 women) enrolled in Cohorts 1 to 3, who underwent at least one post-baseline amyloid PET scan. A significant dose-dependent amyloid plaque lowering was observed across all three analyzed dose levels. In Cohort 3 (1.8 mg/kg) a strong amyloid plaque reduction and amyloid PET negativity in several participants (cut off: <24 Centiloids) were documented already at day 78.
Trontinemab administered with a dose of 1.8 mg/kg led to three times faster and more extensive amyloid lowering than other standard monoclonal antibodies with good safety and low ARIA risk. This is expected to translate into superior clinical efficacy.
Reaching the amyloid negative threshold (or going below the amyloid negative threshold value) is understood to translate into a clinical effect; for example a 70% amyloid negativity rate after 28 weeks of treatment with a dosing every four weeks is understood to translate into a 44% CDR-SB RR versus placebo at 18 months (Clinical Dementia Rating Sum of Boxes Relative Reduction), whereas a 90% amyloid negativity rate after 28 weeks of treatment with an every four weeks dosing is understood to translate into 50% or more CDR-SB RR versus placebo at 18 months (Scelsi et al, CTAD 2023).

| % below | Model predicted |
|---|---|
| threshold | CDR-SB RR |
| 50 | 33.4% |
| 60 | 38.5% |
| 70 | 43.6% |
| 80 | 48.8% |
| 90 | 53.9% |

The participants of the different study Cohorts had the following characteristics:

| | Cohort 1 | | Cohort 2 | | Cohort 3 | | Cohort 4 | |
|---|---|---|---|---|---|---|---|---|
| baseline | 0.2 mg/ | | 0.6 mg/ | | 1.8 mg/ | | 3.6 mg/ | |
| demographic | kg or | | kg or | | kg or | | kg or | |
| and disease | placebo | | placebo | | placebo | | placebo | |
| characteristics | (n = 14) | | (n = 14) | | (n = 16) | | (n = 15) | |
| age, mean | 70.0 | (7.4) | 68.6 | (9.2) | 72.4 | (8.0) | 71.9 | (5.3) |
| (SD) [years] | | | | | | | | |
| sex, female, n | 12 | (85.7%) | 7 | (50.0%) | 10 | (62.5%) | 9 | (60%) |
| [%] | | | | | | | | |
| white skin | 14 | (100%) | 14 | (100%) | 16 | (100%) | 14 | (93.3%) |
| color, n [%] | | | | | | | | |
| CDR-GS, n | | | | | | | | |
| [%] | | | | | | | | |
| 0.5 | 4 | (28.6%) | 6 | (42.9%) | 8 | (50.0%) | 7 | (50.0%) |
| 1 | 6 | (42.9%) | 8 | (57.1%) | 7 | (43.8%) | 7 | (50.0%) |
| 2 | 4 | (28.6%) | 0 | | 1 | (6.2%) | 0 | |
| CDR-SB, | 5.8 | (2.8) | 4.8 | (1.9) | 5.3 | (2.9) | 4.8 | (1.4) |
| mean (SD) | | | | | | | | |
| MMSE, mean | 20.9 | (3.2) | 20.4 | (4.7) | 19.8 | (2.8) | 20.7 | (2.4) |
| (SD) | | | | | | | | |
| ApoE ε4 | | | | | | | | |
| number of | | | | | | | | |
| allels, n [%] | | | | | | | | |
| 0 | 4 | (28.6%) | 7 | (50.0%) | 6 | (37.5%) | 5 | (33.3%) |
| 1 | 7 | (50.0%) | 6 | (42.9%) | 8 | (50.0%) | 7 | (46.7%) |

| | | | | | | | |
|---|---|---|---|---|---|---|---|
| 2 | 3 | (21.4%) | 0 | 2 | (12.5%) | 3 | (30.0%) |

| | | | | | |
|---|---|---|---|---|---|
| missing data | 0 | 1 | (7.1%) | 0 | 0 |

The multiple-dose pharmacodynamics of trontinemab showing mean change from baseline in amyloid PET Centiloids by nominal visit are as follows. For participants on active treatment, the mean change from baseline determined 28 days after administration of Dose 7 was −20.2 Centiloids (reduction by 20.2 Centiloids from baseline; range: −49.4 to −0.7) in the 0.2 mg/kg Cohort and −27.6 Centiloids (reduction by 27.6 Centiloids from baseline; range: −52.3 to −1.4) in the 0.6 mg/kg Cohort (mean number of doses received: 6.6 and 6.2, respectively). In the Cohort receiving 1.8 mg/kg, a more pronounced mean change from baseline was seen based on the data at the time of the data snapshot. The mean change from baseline determined 22 days after administration of nominal Dose 3 was −65.3 Centiloids (reduction by 65.3 Centiloids from baseline; range: −102.8 to −42.6) in the 1.8 mg/kg Cohort (after a mean number of doses received: of 2.8).

| dose Q4W | number | mean number | mean change | 90% |
|---|---|---|---|---|
| 28 days after | of | of received | from baseline | confidence |
| Dose 7 | participants | doses | in Centiloids | interval |
| 0 mg/kg | 7 | 7 | −1 | +/−15 |
| 0.2 mg/kg | 10 | 6.6 | −20 | +/−7 |
| 0.6 mg/kg | 9 | 6.2 | −28 | +/−9 |
| dose Q4W | number | mean number | mean change | 90% |
| 22 days after | of | of received | from baseline | confidence |
| Dose 3 | participants | doses | in Centiloids | interval |
| 0 mg/kg | 3 | 3 | +9 | +/−22 |
| 1.8 mg/kg | 9 | 2.8 | −65 | +/−13 |

Thus, it has surprisingly been found that rapid amyloid plaque clearance is achieved at significantly lower dose levels than with typical anti-amyloid monoclonal antibodies.

These results show that trontinemab rapidly lowers amyloid plaques burden in a dose-dependent fashion. In a majority of patients, the amyloid plaque load is reduced to a level comparable to people not suffering from AD and in short time.

In more detail, it has been found that a monthly intravenous dose of 1.8 mg/kg led to a rapid and substantial amyloid lowering of 84 Centiloids (−84 Centiloids from baseline) after 7 months. This is faster than with standard anti-Abeta monoclonal antibodies; 72% of the participants were below the Abeta protein positivity threshold (24.1 CL) at week 28.

In more detail, it has been found that a monthly intravenous dose of 3.6 mg/kg led to a rapid and substantial amyloid lowering of 91 Centiloids (−91 CL from baseline) after 3 months (n=8). This is ˜6 times faster than with standard anti-Abeta monoclonal antibodies; 6/8 (63%) participants were below the Abeta protein positivity threshold (24.1 CL) at week 12.

The materially improved safety and tolerability profile of trontinemab, relative to standard anti-Abeta monoclonal antibodies, was confirmed with this data. Infusion-related reactions (IRRs) remain the most prominent side effect (7/15; 46.7%) with common symptoms of chills, fever and headache and can be mitigated by appropriate premedication, in one embodiment with a pre-medication with corticosteroids or pre-medication with acetaminophen/NSAID and dexamethasone. NSAID=non-steroidal anti-inflammatory drug.

None of the Cohort 4 participants showed ARIA-E so far (the overall ARIA-E rate in the study is 2/59—both cases were radiographically mild and reversible). A transient mild anemia was observed in 5 participants in Cohort 3 and in one participant in Cohort 4. Trends of decreasing mean hemoglobin levels and decreasing red blood cell counts were recorded in all treatments groups (including placebo), suggesting that frequent blood collection likely significantly contributed to the anemia phenotype.

Thus, in one embodiment, trontinemab has a total ARIA-E rate of less than 13% of treated subjects and a symptomatic ARIA-E rate of 3% or less of treated subjects. This low ARIA-E rate translates into a short ARIA-E risk period due to faster amyloid removal.

It was found that 4 of 11 participants (36%) were below the amyloid positivity threshold of 24.1 Centiloids at week 11. At week 28 already 6 of 8 participants (75%) were below the amyloid positivity threshold of 24.1 Centiloids and 5 of 8 participants (63%) were below 11 Centiloids. In contrast thereto, participants receiving doses of 0.2 mg/kg and 0.6 mg/kg had mean amyloid values in Centiloids at week 28 of 74 and 56, respectively, with none of the participants in the 0.2 mg/kg dosing group and only one of the participants in the 0.6 mg/kg dosing group being below the amyloid positivity threshold of 24.1 Centiloids.

| | Mean amyloid value in CL | | | | | | | |
|---|---|---|---|---|---|---|---|---|
| | (% amyloid negative (<24.1 CL)) | | | | | | | |
| | | | 0.2 | 0.6 | 1.8 | | 3.6 | |
| | Placebo | | mg/kg | mg/kg | mg/kg | | mg/kg | |
| Baseline | 95-98 | (0%) | 91 (0%) | 88 (0%) | 100 | (0%) | 119 | (0%) |
| Week 11 | 128 | (0) | — | — | 40 | (36) | — | |
| Week 12 | 119 | (0%) | — | — | 40 | (36%) | 21 | (63%) ** |

| | | | | | | | |
|---|---|---|---|---|---|---|---|
| Week 28 | 91 | (0%) | 74 (0%) | 56 (10%) | 9 | (75%) * | — |

* 6/8 (75 % of participants) <24.1 CL, 5/8 (63 %) <11 CL at 1.8 mg/kg after 28 weeks
** 5/8 (63 % of participants) <24.1 CL, 4/8 (50 %) <11 CL at 3.6 mg/kg after 12 weeks

The results for Cohorts 1 to 3 are visualized in FIGS. 3 (mean amyloid PET burden in Centiloids) and 4 (mean amyloid reduction from baseline in Centiloids. The study was a randomized (4:1) with 10 or more participants per group (active:placebo). All participants were of white skin and had a body weight between 60 and 70 kg. The APOE ε4 carrier status was 71% for the dosing of 0.2 mg/kg, 43% for the dosing of 0.6 mg/kg and 63% for the dosing of 1.8 mg/kg.

The results including the results for Cohort 4 after 12 weeks are visualized in FIG. 5 (mean amyloid reduction from baseline in Centiloids). The study was a randomized (4:1) with 10 or more participants per group (active:placebo). All participants were of white skin except for one in Cohort 4 and had a body weight between 68 and 72.5 kg. The APOE ε4 carrier status was about 71% for the dosing of 0.2 mg/kg, about 43% for the dosing of 0.6 mg/kg, about 63% for the dosing of 1.8 mg/kg and about 67% for the dosing of 3.6 mg/kg.

It can be seen that with doses of 1.8 mg/kg and 3.6 mg/kg a reduction by at least 60 Centiloids to more than 90 Centiloids after 12 weeks and by at least 80 Centiloids to more than 90 Centiloids after 28 weeks was achieved.

In the following table, as well as in FIG. 6, the results obtained with trontinemab are shown together with those of other anti-Abeta antibodies. It has to be pointed out that amyloid PET burden is in Centiloids, which is variable across studies. However, lower baseline levels in CLARITY-AD (lecanemab) enabled faster crossing of the amyloid positivity threshold, even though the absolute change was comparable to the GRADUATE studies.

| | | | baseline | amyloid | |
|---|---|---|---|---|---|
| | | | amyloid | PET | |
| | dosing | | PET | change | |
| antibody | schedule | dose | (Centiloids) | (Centiloids) | at |
| trontinemab | Q4W | 1.8 mg/kg (IV) | 96 | −65 | day 78 |
| gantenerumab | Q2W | 510 mg (SC) | 91.8 | −57.6 | week 116 |
| (GRADUATE I) | | (= 7.3 mg/kg | | | |
| | | @ 70 kg bw) | | | |
| gantenerumab | Q2W | 510 mg (SC) | 92.9 | −46.8 | week 116 |
| (GRADUATE II) | | (= 7.3 mg/kg | | | |
| | | @ 70 kg bw) | | | |
| aducanumab | Q4W | 10 mg/kg | 90.8 | −53.5 | week 78 |
| (ENGAGE) | | | | | |
| lecanemab | Q2W | 10 mg/kg | 78 | −55.5 | week 78 |
| (CLARITY AD) | | | | | |
| donanemab | Q4W | 20 mg/kg (IV) | 108 | −70 | week 24 |
| (TRAIL- | | | | −84 | week 76 |
| BLAZER-ALZ) | | | | | |
| aducanumab | Q4W | 10 mg/kg | 85.3 | −64.2 | week 78 |
| (EMERGE) | | | | | |

bw = body weight

It has to be expressly pointed out that one third of the patients were PET negative already after 12 weeks (every four weeks dosing; Q4W) of administration of trontinemab at a dose of 1.8 mg/kg or 3.6 mg/kg.

The following events occurred during the study until Oct. 23, 2023.

| Total number of | Cohort 1 | Cohort 2 | Cohort 3 | Cohort 4 |
|---|---|---|---|---|
| participants with at | 0.2 mg/kg | 0.6 mg/kg | 1.8 mg/kg | 3.6 mg/kg |
| least one event, | or placebo | or placebo | or placebo | or placebo |
| (%) | (n = 14) | (n = 14) | (n = 16) | (n = 15) |
| infusion related | 1 (1.7 %) | 4 (28.6 %) | 12 (75.0%) | 7 (46.7%) |
| reaction (IRR) * | | | | |
| Anemia ** | 2 (14.3 %) | 0 | 5 (31.2%) | 1 (6.7%) |

* Common IRR symptoms include fever, chills, and headache. In Cohorts 1-3, most IRRs occurred after administration of the first study drug dose (without premedication), were mild to moderate in severity and resolved with or without appropriate medication. Subsequently, routine premedication with paracetamol/nonsteroidal anti-inflammatory drugs was implemented in Cohorts 3 and 4, which reduced the incidence and symptoms of IRRs.
** A transient mild anemia was observed in 5 participants in Cohort 3 and in one participant in Cohort 4. Trends of decreasing mean hemoglobin levels and decreasing red blood cell counts were recorded in all treatments groups (including placebo), suggesting that frequent blood collection likely significantly contributed to the anemia phenotype.

Only two cases of ARIA occurred so far in the Cohort with 1.8 mg/kg dosing in the 12 participants (3 placebo, 9 dosed) (1 amyloid related imaging abnormalities-edema (ARIA-E), mild, resolved within 1 week without medication; 1 amyloid related imaging abnormalities-hemosiderosis (ARIA-H), asymptomatic).

Abeta protein plaque decoration correlates with cumulative doses and, thus, TfR1 transport across the BBB is not desensitized by multiple dosing.

The ARIA event data is shown in the following table with the number of participants with events listed.

| Total number of | Cohort 1 | Cohort 2 | Cohort 3 | Cohort 4 |
|---|---|---|---|---|
| participants with | 0.2 mg/ | 0.6 mg/ | 1.8 mg/ | 3.6 mg/ |
| event [events | kg or | kg or | kg or | kg or |
| per participant], | placebo | placebo | placebo | placebo |
| (%) | (n = 14) | (n = 14) | (n = 16) | (n = 15) |
| ARIA-E * | 0 | 0 | 1 (two times) | 0 |
| | | | (6.7%) | |
| ARIA-H ** | 0 | 0 | 0 | 0 |
| microhemorrhage | | | | |
| leptomening-eal | 0 | 0 | 1 (two times) | 0 |
| hemosider-osis | | | (6.7 %) | |
| (LH) | | | | |
| ARIA-E with | 0 | 0 | 0 | 0 |
| concurrent | | | | |
| ARIA-H | | | | |
| Macrohemorrhage | 0 | 0 | 0 | 0 |

* One participant in Cohort 3 developed two episodes of ARIA-E: first, on routine Day 22 MRI scan, radiographically mild, temporally associated with mildly impaired attention over approximately one week, complete radiographic resolution within 4 weeks; second, on routine on Day 281 MRI, radiographically mild+, asymptomatic, complete radiographic resolution within 8 weeks.
** One participant in Cohort 3 developed 2 asymptomatic ARIA-H findings not concurrent with ARIA-E; one left occipital LH (12 mm) on routine Day 162 MRI, then one right frontal LH (8 mm) on routine Day 281 MRI.

1.-14. (canceled)

15. A method of reducing amyloid level in a subject comprising administering a composition comprising a bispecific antibody specifically binding to human Abeta protein and human transferrin receptor (bispecific anti-Abeta/TfR antibody),
wherein the composition is administered intravenously at a dose of 0.2 mg/kg to 7.2 mg/kg once every four weeks, and
wherein the bispecific anti-Abeta/TfR antibody comprises
i) two copies of a first pair of an antibody heavy chain variable domain and a cognate antibody light chain variable domains forming a binding site specifically binding to human Abeta protein comprising
three heavy chain complementarity determining regions (HCDRs) comprising amino acid sequences of SEQ ID NO: 1 (HCDR1), SEQ ID NO: 2 (HCDR2), and SEQ ID NO: 3 (HCDR3); and
three light chain complementarity determining regions (LCDRs) comprising amino acid sequences of SEQ ID NO: 5 (LCDR1), SEQ ID NO: 6 (LCDR2), and SEQ ID NO: 7 (LCDR3); and
ii) one copy of a second pair of an antibody heavy chain variable domain and a cognate antibody light chain variable domains forming a binding site specifically binding to human transferrin receptor comprising
three heavy chain complementarity determining regions (HCDRs) comprising amino acid sequences of SEQ ID NO: 9 (HCDR1), SEQ ID NO: 10 (HCDR2), and SEQ ID NO: 11 (HCDR3); and
three light chain complementarity determining regions (LCDRs) comprising amino acid sequences of SEQ ID NO: 13 (LCDR1), SEQ ID NO: 14 (LCDR2), and SEQ ID NO: 15 (LCDR3).

16. The method of claim 15, wherein
i) administration of the composition results in the subject being amyloid negative as determined by visual reads of amyloid PET images,
ii) administration of the composition reduces the amyloid level of the subject by at least 40 Centiloids or at least 60 Centiloids as determined by visual reads of amyloid PET:
iii) administration of the composition reduces the amyloid level of the subject within 3 months after the start of the administration of the composition; and/or
iv) administration of the composition reduces the severity of at least one symptom associated with Alzheimer's disease in a subject.

17. The method of claim 16, wherein
i) the amyloid negative determination is made after 3 months of administration of the composition;
ii) the at least one symptom associated with Alzheimer's Disease is determined by CDR-SB, CDR-GS, MMSE, ADCOMS, and/or PET; and/or
iii) the at least one symptom associated with Alzheimer's Disease is chosen from clinical decline and brain amyloid level.

18. (canceled)

19. The method of claim 15, wherein the bispecific anti-Abeta/TfR antibody is trontinemab.

20. The method of claim 15, wherein
i) the subject has been diagnosed as having mild or prodromal Alzheimer's disease;
ii) the subject does not exhibit severe amyloid related imaging abnormalities-edema (ARIA-E) or severe amyloid related imaging abnormalities-hemosiderosis (ARIA-H).

21. The method of claim 15, wherein the composition is administered at a dose of 1.8 mg/kg to 7.2 mg/kg or of 1.8 mg/kg to 3.6 mg/kg.

22.-25. (canceled)

26. The method of claim 15, wherein no further Alzheimer's disease medication is administered to the subject.

27. (canceled)

28. The method of claim 15, wherein the composition is, after a number of administrations of the dose, continued to be administered at a lower dose of 0.2 mg/kg to 3.6 mg/kg relative to the weight of the subject.

29. The method of claim 28, wherein the number of administrations is 12 to 36.

30.-33. (canceled)

34. The method of claim 17, wherein the subject's clinical decline is assessed using CDR-SB, CDR-GS, MMSE, ADCOMS, and/or ADAS-cog.

35. The method of claim 15, wherein the CSF/plasma ratio of the antibody in the subject is 0.5% to 1.2%.

36. A method of reducing the severity of at least one symptom associated with Alzheimer's disease in a subject, wherein the at least one symptom is reduced relative to the severity of the same symptom in the same subject prior to treatment, comprising administering a composition comprising a bispecific antibody specifically binding to human Abeta protein and human transferrin receptor (bispecific anti-Abeta/TfR antibody),
wherein the antibody is administered intravenously at a dose of 0.2 mg/kg to 7.2 mg/kg once every four weeks, and
wherein the bispecific anti-Abeta/TfR antibody comprises
i) two copies of a first pair of an antibody heavy chain variable domain and a cognate antibody light chain variable domains forming a binding site specifically binding to human Abeta protein comprising three heavy chain complementarity determining regions (HCDRs) comprising amino acid sequences of SEQ ID NO: 1 (HCDR1), SEQ ID NO: 2 (HCDR2), and SEQ ID NO: 3 (HCDR3); and three light chain complementarity determining regions (LCDRs) comprising amino acid sequences of SEQ ID NO: 5 (LCDR1), SEQ ID NO: 6 (LCDR2), and SEQ ID NO: 7 (LCDR3); and
ii) one copy of a second pair of an antibody heavy chain variable domain and a cognate antibody light chain variable domains forming a binding site specifically binding to human transferrin receptor comprising three heavy chain complementarity determining regions (HCDRs) comprising amino acid sequences of SEQ ID NO: 9 (HCDR1), SEQ ID NO: 10 (HCDR2), and SEQ ID NO: 11 (HCDR3); and three light chain complementarity determining regions (LCDRs) comprising amino acid sequences of SEQ ID NO: 13 (LCDR1), SEQ ID NO: 14 (LCDR2), and SEQ ID NO: 15 (LCDR3).

37.-40. (canceled)

41. The method of claim 36, wherein the bispecific anti-Abeta/TfR antibody is trontinemab.

42. The method of claim 36, wherein
i) the subject has been diagnosed as having mild or prodromal Alzheimer's disease- and/or
ii) the subject does not exhibit severe amyloid related imaging abnormalities-edema (ARIA-E) or severe amyloid related imaging abnormalities-hemosiderosis (ARIA-H).

43. The method of claim 36, wherein the composition is administered at a dose of 1.8 mg/kg to 7.2 mg/kg or of 1.8 mg/kg to 3.6 mg/kg.

44.-47. (canceled)

48. The method of claim 36, wherein no further Alzheimer's disease medication is administered to the subject.

49. (canceled)

50. The method of claim 36, wherein the composition is, after a number of administrations of the dose, continued to be administered at a lower dose of 0.2 mg/kg to 3.6 mg/kg relative to the weight of the subject.

51. The method of claim 50, wherein the number of administrations is 12 to 36.

52. (canceled)

53. (canceled)

54. The method of claim 36, wherein the at least one symptom associated with Alzheimer's disease is chosen from clinical decline and brain amyloid level.

55. The method of claim 36, wherein the CSF/plasma ratio of the antibody in the subject is 0.5% to 1.2%.

56. A method of reducing clinical decline in a subject comprising administering a composition comprising a bispecific antibody specifically binding to human Abeta protein and human transferrin receptor (bispecific anti-Abeta/TfR antibody),
wherein the antibody is administered intravenously at a dose of 0.2 mg/kg to 7.2 mg/kg once every four weeks, and
wherein the bispecific anti-Abeta/TfR antibody comprises
i) two copies of a first pair of an antibody heavy chain variable domain and a cognate antibody light chain variable domains forming a binding site specifically binding to human Abeta protein comprising three heavy chain complementarity determining regions (HCDRs) comprising amino acid sequences of SEQ ID NO: 1 (HCDR1), SEQ ID NO: 2 (HCDR2), and SEQ ID NO: 3 (HCDR3); and three light chain complementarity determining regions (LCDRs) comprising amino acid sequences of SEQ ID NO: 5 (LCDR1), SEQ ID NO: 6 (LCDR2), and SEQ ID NO: 7 (LCDR3); and
ii) one copy of a second pair of an antibody heavy chain variable domain and a cognate antibody light chain variable domains forming a binding site specifically binding to human transferrin receptor comprising three heavy chain complementarity determining regions (HCDRs) comprising amino acid sequences of SEQ ID NO: 9 (HCDR1), SEQ ID NO: 10 (HCDR2), and SEQ ID NO: 11 (HCDR3); and three light chain complementarity determining regions (LCDRs) comprising amino acid sequences of SEQ ID NO: 13 (LCDR1), SEQ ID NO: 14 (LCDR2), and SEQ ID NO: 15 (LCDR3).

57.-61. (canceled)

62. The method of claim 56, wherein the subject has been diagnosed as having mild or prodromal Alzheimer's disease.

63. The method of claim 56, wherein the composition is administered at a dose of 1.8 mg/kg to 7.2 mg/kg or of 1.8 mg/kg to 3.6 mg/kg.

64.-67. (canceled)

68. The method of claim 56, wherein no further Alzheimer's disease medication is administered to the subject.

69. (canceled)

70. The method of claim 56, wherein the composition is, after a number of administrations of the dose, continued to be administered at a lower dose of 0.2 mg/kg to 3.6 mg/kg relative to the weight of the subject.

71. The method of claim 70, wherein the number of administrations is 12 to 36.

72. (canceled)

73. The method of claim 56, wherein the subject's clinical decline is assessed using CDR-SB, CDR-GS, MMSE, ADCOMS, and/or ADAS-cog.

74. The method of claim 56, wherein the CSF/plasma ratio of the antibody in the subject is 0.5% to 1.2%.

75. A method of exposing a subject's brain tissue to an antibody comprising administering a composition comprising a bispecific antibody specifically binding to human Abeta protein and human transferrin receptor (bispecific anti-Abeta/TfR antibody),
wherein the antibody is administered intravenously at a dose of 0.2 mg/kg to 7.2 mg/kg once every four weeks,
wherein the CSF/plasma ratio of the antibody in the subject is 0.5% to 1.2%, and
wherein the bispecific anti-Abeta/TfR antibody comprises
i) two copies of a first pair of an antibody heavy chain variable domain and a cognate antibody light chain variable domains forming a binding site specifically binding to human Abeta protein comprising three heavy chain complementarity determining regions (HCDRs) comprising amino acid sequences of SEQ ID NO: 1 (HCDR1), SEQ ID NO: 2 (HCDR2), and SEQ ID NO: 3 (HCDR3); and three light chain complementarity determining regions (LCDRs) comprising amino acid sequences of SEQ ID NO: 5 (LCDR1), SEQ ID NO: 6 (LCDR2), and SEQ ID NO: 7 (LCDR3); and
ii) one copy of a second pair of an antibody heavy chain variable domain and a cognate antibody light chain variable domains forming a binding site specifically binding to human transferrin receptor comprising three heavy chain complementarity determining regions (HCDRs) comprising amino acid sequences of SEQ ID NO: 9 (HCDR1), SEQ ID NO: 10 (HCDR2), and SEQ ID NO: 11 (HCDR3); and three light chain complementarity determining regions (LCDRs) comprising amino acid sequences of SEQ ID NO: 13 (LCDR1), SEQ ID NO: 14 (LCDR2), and SEQ ID NO: 15 (LCDR3).

76.-80. (canceled)

81. The method of claim 75, wherein the subject has been diagnosed as having mild or prodromal Alzheimer's disease.

82. The method of claim 75, wherein the composition is administered at a dose of 1.8 mg/kg to 7.2 mg/kg or of 1.8 mg/kg to 3.6 mg/kg.

83. (canceled)

84. The method of claim 75, wherein the administration of the composition reduces the amyloid level of the subject by at least 40 Centiloids as determined by visual reads of amyloid PET.

85. The method of claim 84, wherein the administration of the composition reduces the amyloid level of the subject by at least 60 Centiloids as determined by visual reads of amyloid PET.

86. The method of claim 75, wherein the administration of the composition reduces the amyloid level of the subject within 3 months after the start of the administration of the composition.

87. The method of claim 75, wherein no further Alzheimer's disease medication is administered to the subject.

88. (canceled)

89. The method of claim 75, wherein the composition is, after a number of administrations of the dose, continued to be administered at a lower dose of 0.2 mg/kg to 3.6 mg/kg relative to the weight of the subject.

90. The method of claim 89, wherein the number of administrations is 12 to 36.

91.-95. (canceled)

96. A formulation comprising a bispecific antibody specifically binding to human Abeta protein and human transferrin receptor (bispecific anti-Abeta/TfR antibody),
wherein the bispecific anti-Abeta/TfR antibody comprises
i) two copies of a first pair of an antibody heavy chain variable domain and a cognate antibody light chain variable domains forming a binding site specifically binding to human Abeta protein comprising three heavy chain complementarity determining regions (HCDRs) comprising amino acid sequences of SEQ ID NO: 1 (HCDR1), SEQ ID NO: 2 (HCDR2), and SEQ ID NO: 3 (HCDR3); and three light chain complementarity determining regions (LCDRs) comprising amino acid sequences of SEQ ID NO: 5 (LCDR1), SEQ ID NO: 6 (LCDR2), and SEQ ID NO: 7 (LCDR3); and
ii) one copy of a second pair of an antibody heavy chain variable domain and a cognate antibody light chain variable domains forming a binding site specifically binding to human transferrin receptor comprising three heavy chain complementarity determining regions (HCDRs) comprising amino acid sequences of SEQ ID NO: 9 (HCDR1), SEQ ID NO: 10 (HCDR2), and SEQ ID NO: 11 (HCDR3); and three light chain complementarity determining regions (LCDRs) comprising amino acid sequences of SEQ ID NO: 13 (LCDR1), SEQ ID NO: 14 (LCDR2), and SEQ ID NO: 15 (LCDR3); and
wherein the bispecific anti-Abeta/TfR antibody has a concentration of about 1 mg/mL to about 200 mg/mL.

97. The formulation of claim 96, wherein the bispecific anti-Abeta/TfR antibody has a concentration of about 1 mg/mL to about 20 mg/mL.

98. The formulation of claim 96, wherein the bispecific anti-Abeta/TfR antibody has at a concentration of about 150 mg/mL.

99. The formulation of claim 96, wherein the formulation further comprises a poloxamer, a buffer, a stabilizer, a surfactant, a sugar, a chelating agent, or a combination thereof.

100. The formulation of claim 96, wherein the formulation has a pH value of about pH 4.5 to about pH 6.0.

101. The formulation of claim 96, wherein the bispecific anti-Abeta/TfR antibody is trontinemab.

102. A method of reducing amyloid level in a subject comprising administering the formulation of claim 96, wherein the formulation is administered intravenously at a dose of 0.2 mg/kg to 7.2 mg/kg once every four weeks.

103. A method of reducing the severity of at least one symptom associated with Alzheimer's disease in a subject, wherein the at least one symptom is reduced relative to the severity of the same symptom in the same subject prior to treatment, comprising administering the formulation of claim 96, wherein the formulation is administered intravenously at a dose of 0.2 mg/kg to 7.2 mg/kg once every four weeks.

104. A method of reducing clinical decline in a subject comprising administering the formulation of claim 96, wherein the antibody is administered intravenously at a dose of 0.2 mg/kg to 7.2 mg/kg once every four weeks.

105. A method of exposing a subject's brain tissue to an antibody comprising administering the formulation of claim 96, wherein the antibody is administered intravenously at a dose of 0.2 mg/kg to 7.2 mg/kg once every four weeks.

106. The method of claim 105, wherein the formulation of claim 96 is, after a number of administrations of the dose, continued to be administered at a lower dose of 0.2 mg/kg to 3.6 mg/kg relative to the weight of the subject.

107. The method of claim 106, wherein the number of administrations is 12 to 36.

108. A composition comprising the formulation of claim 96 and a package insert.
